(19) United States
(12) Patent Application Publication
(10) Pub. No.: US 2006/0251576 A1
(43) Pub. Date: Nov. 9, 2006

(54) Methods for measuring cholesterol metabolism and transport

(75) Inventor: Marc K. Hellerstein, Kensington, CA (US)

Correspondence Address: MORRISON & FOERSTER LLP, 425 MARKET STREET, SAN FRANCISCO, CA, 94105-2482, US

(73) Assignee: The Regents of the University of California, Oakland, CA (US)

(21) Appl. No.: 11/416,842

(22) Filed: May 2, 2006

Related U.S. Application Data
(60) Provisional application No. 60/677,672, filed on May 3, 2005.

(51) Int. Cl. A61K 51/00; A61K 49/00

(52) U.S. Cl. 424/1.11; 424/9.2

(57) ABSTRACT

The present invention relates to biochemical methods for determining reverse cholesterol transport (RCT). Specifically, the three components of RCT (efflux, plasma, and excretion) are measured in vivo by administering an isotope-labeled cholesterol or cholesterol-related molecule or cholesterol-related complex, and then measuring the dilution or appearance of isotopes in the various cholesterol or cholesterol-related molecules or cholesterol-related complexes, as well as recovery in sterol end-products, that are part of RCT. A parameter of Global RCT flux, representing for the first time in living organisms that combined rate of cholesterol efflux from tissues into blood and excretion from blood out of the body, is generated. Such methods find use in drug discovery and development, diagnosis and prognosis of atherosclerosis and other blood vessel diseases and conditions, the selection of proper doses for treating disease, and selecting subjects for therapies targeting RCT flux.

### CROSS-REFERENCE TO RELATED APPLICATION

This application claims priority to U.S. patent application 60/677,672 filed on May 3, 2005, which is hereby incorporated by reference in its entirety.

### FIELD OF THE INVENTION

The present invention relates to the field of cholesterol metabolism. In particular, methods for quantitatively measuring cholesterol metabolism and transport, with emphasis on reverse cholesterol transport, are described.

### BACKGROUND OF THE INVENTION

Atherosclerosis, the most common form of arteriosclerosis, is a disease of large and medium-sized arteries (e.g., coronary, carotid, and lower extremity arteries), and of the elastic arteries, such as the aorta and iliac vessels. The atheroma, or fibrofatty plaque within the intima that consists of a lipid core and fibrous cap, is pathognomonic (Robbins Pathologic Basis of Disease 557 (Cotran et al. eds., 4th ed. 1989)). In addition to being a primary risk factor for myocardial and cerebral infarcts, atherosclerosis is responsible for such medical conditions as chronic lower extremity ischemia and gangrene, and for mesenteric occlusion. Despite a recent reduction in mortality from coronary heart disease, about 50% of all deaths in the United States are still attributed to atherosclerosis (Scientific American Medicine §1 (Rubenstein et al. eds., 1991)).

Epidemiologic, postmortem, and angiographic studies have firmly established a causal relationship between elevated serum cholesterol levels and the genesis of atherosclerosis (Levine et al., Cholesterol Reduction In Cardiovascular Disease, N Eng J Med 332(8):512-521 (1995)). Although there is no single level of plasma cholesterol that identifies those at risk, in general, the higher the level, the higher the risk. However, the risk rises significantly with cholesterol levels above 200 mg/dl (Robbins Pathologic Basis of Disease, supra, at 559). Levels of total cholesterol are typically classified as being desirable (<200 mg/dl), borderline high (200-239 mg/dl), or high (≧240 mg/dl). Dietary treatment is usually recommended for subjects with high risk levels of low density lipoprotein (LDL) cholesterol and for those with borderline-high risk levels who have at least two additional risk factors for atherosclerosis (e.g., hypertension, diabetes mellitus, cigarette smoking, etc.). However, dietary therapy has been found to be effective only in subjects whose diets were higher than average in cholesterol and saturated fats (Adult Treatment Panel II. National Cholesterol Education Program: Second Report of the Expert Panel on Detection, Evaluation, and Treatment of High Blood Cholesterol in Adults, Circulation 89:1333-1445 (1994)), and would be ineffective in subjects with a genetic predisposition to hypercholesterolemia. In the case of persistent high cholesterol levels, drug therapy may be prescribed.

Currently marketed drugs for the treatment of hypercholesterolemia work by such methods as inhibiting de novo cholesterol synthesis and/or stimulating clearance of low density lipoprotein (LDL) cholesterol by the LDL receptor (e.g., lovastatin, other statins—see FIG. 1), decreasing the production of very low density lipoprotein (VLDL) (e.g., gemfibrozil), or by inhibiting bile acid reabsorption in the intestines (e.g., cholestyramine). Examination of cholesterol metabolism, however, also reveals that the process of reverse cholesterol transport (RCT) allows a pathway through which cholesterol may be removed from tissues and may exit the body. At present, there is no known method for measuring the rate of cholesterol flow through the RCT pathway from tissue to excretion in a living organism.

RCT is a biological pathway through which cholesterol is mobilized and transported out of cells and out of the body (FIG. 1). RCT can be divided into three components: (1) efflux of cholesterol from tissues into blood (the “efflux component”); (2) transport and distribution of cholesterol in the plasma (the “plasma component”); and (3) excretion via the liver or intestine into the feces (the “hepatic component” or “excretory component”). Cholesterol is incorporated into bile secretions either as bile acids or free cholesterol, which are then secreted into the intestinal lumen, a portion of which leaves the body in the stool. Sterols can also be directly released by intestinal tissues into the gut lumen, with subsequent excretion of a portion. These RCT pathways represent the only significant mechanism by which cholesterol can be removed from the body.

It would therefore be desirable to have methods that allow for the measurement of these steps of RCT. From the functional point-of-view, the efflux component and the hepatic or excretory component are the key steps, as these represent the exit of cholesterol from cells and from the body, respectively. A way of measuring RCT, and these steps in particular, would allow pharmaceutical companies and other drug developers to screen for agents or candidate therapies that modulate the efflux of cholesterol out of tissues and out of the body to the subject's benefit; would allow clinicians to select optimal doses of agents affecting RCT; would allow clinicians to diagnose and monitor the progression of cholesterol-related disease; would allow clinicians to assess the risk for cholesterol-related disease; would allow clinicians to select subjects or identify subject groups that would respond to RCT-based candidate therapies; and would allow for the detailed study of the mechanisms of cholesterol related disease. Such methods are disclosed herein.

### SUMMARY OF THE INVENTION

To meet these needs, the present invention provides methods for determining the rates of cholesterol metabolism and transport, enabling the measurement of RCT in humans and experimental animals.

In one aspect, the efflux component of RCT may be determined in a subject. One or more isotopically-labeled cholesterol or cholesterol-related molecules or cholesterol-related complexes are administered to the subject at a known rate. After a period of time, one or more biological samples containing the cholesterol or cholesterol-related molecules or cholesterol-related complexes are then obtained from the subject. The isotopic content or isotopic pattern of the cholesterol or cholesterol-related molecules or cholesterol-related complexes is then measured. The dilution of the administered labeled molecules by corresponding endogenous unlabeled molecules is then calculated to determine the activity or rate of the efflux component (rate of appearance [Ra]) of RCT in the subject. The administered molecule may be isotope-labeled free cholesterol, and may be infused intravenously at a steady state, and the dilution of the labeled free cholesterol in plasma may be determined by measuring the isotopic content or isotopic pattern of plasma cholesterol at many points spaced out over the duration of the infusion.

In another aspect, the activity (e.g., the rate of transport, the mass of cholesterol or cholesterol-related molecule or cholesterol-related complex transported or converted, or the fraction of bile acids or neutral sterols derived from RCT) of the hepatic or excretory component of RCT may be determined. One or more isotopically-labeled cholesterol or cholesterol-related molecules or cholesterol-related complexes are administered to a subject. After a period of time, one or more samples containing bile acids, fecal neutral sterols, or cholesterol or cholesterol-related molecules or cholesterol-related complexes are obtained from the subject. The isotopic content or isotopic pattern in bile acids, excreted neutral sterols incorporated from cholesterol, cholesterol-related molecules or cholesterol-related complexes is then measured. The incorporation of isotope label via the hepatic or excretory component of RCT is then calculated to determine the activity or rate of the hepatic or excretory component of RCT in the subject. Measurement of the hepatic or excretory component of RCT as described above may be carried out in part by measuring the incorporation of isotope label from intravenously-administered isotopically-labeled free cholesterol into bile acids, and may be accompanied by a measurement of bile acid pool size. Bile acid pool size may be measured by administering known amounts of one or more isotopically-labeled bile acids to the subject, and, after a period of time, obtaining samples from the subject containing bile acids, measuring the isotopic content, isotopic pattern or rate of change in the isotopic content or isotopic pattern of the bile acids in the sample, and calculating the dilution of the administered labeled bile acid by endogenous bile acids, yielding a measurement of total bile acid pool size in the system. Suitable bile acids for this aspect of the invention include, but are not limited to, cholic acid, chenodeoxycholic acid, deoxycholic acid, and lithocholic acid. The bile acid may be cholic acid.

In another aspect of the disclosure, it has been discovered that, by combining the efflux/mobilization rate (efflux component) with the efficiency of label recovery in fecal sterols, (hepatic or excretory component), a “Global RCT” parameter can be measured. Global RCT flux represents the cholesterol efflux from tissues that ends up excreted as fecal sterols, or the number of cholesterol molecules entering the plasma pool/day that are recovered as fecal sterols. This Global RCT metric provides, for the first time, an integrated measure of the cholesterol flux from tissues to stool in a living organism, including human subjects. It can be understood by those of skill in the art that this metric represents the cholesterol flux rate from tissues through the bloodstream and out of the body—the definition of global RCT. This metric can be calculated by multiplying the efflux rate (Ra) by the excretory efficiency.

In yet another aspect of the invention, the plasma component of RCT is determined. One or more isotopically-labeled cholesterol or cholesterol-related molecules or cholesterol-related complexes are administered to the subject. After a period of time, one or more plasma samples containing cholesterol or cholesterol-related molecules or cholesterol-related complexes of interest are obtained from the subject, and the isotopic content or isotopic pattern of the cholesterol or cholesterol-related molecules or cholesterol-related complexes is then measured. The transport or metabolism of cholesterol or cholesterol-related molecules or cholesterol-related complexes through different portions (see FIG. 2) of the plasma component of RCT is then determined by the disappearance or appearance of isotope label in the various cholesterol or cholesterol-related molecules or cholesterol-related complexes.

The methods of the present invention may be applied to assess the effect of candidate therapies on RCT. The method involves administering the candidate therapy to a subject, comparing any or all of the components of RCT in the subject before and after administration of the candidate therapy or in comparison to matched subjects who have not received the candidate therapy or to historical data, and calculating the difference in RCT before and after administration of the candidate therapy or with and without the candidate therapy. The candidate therapy may be a single agent or compound. Alternatively, the candidate therapy may be a combination of agents or compounds. The candidate therapy also may be a single agent or compound or a combination of agents or compounds together with some other intervention, such as a lifestyle change (e.g., change in diet, increase in exercise).

In another variation, the effect of dietary modification on the risk for atherosclerosis is assessed by comparing the rate of RCT in the subject before and after dietary modification, and calculating the difference in the rate of RCT before and after dietary modification.

In yet a further variation, kits for determining the rate of RCT are provided. The kits may include labeled cholesterol or cholesterol-related molecules or cholesterol-related complexes, labeled bile acids, or a combination thereof, and instructions for use of the kit. The kit may optionally also include tools for administration of said labeled cholesterol or cholesterol-related molecules or cholesterol-related complexes, or labeled bile acids to the subject and instruments for collecting samples from the subject.

In a further variation, a method for determining the molecular flux rate of the hepatic or excretory component of reverse cholesterol transport (RCT) in a living system is described. The method may include: a) administering an isotopically labeled cholesterol molecule or isotopically labeled cholesterol-related molecule to the living system; b) obtaining a sample from the living system wherein the sample include one or more isotopically labeled cholesterol molecules, bile acids or excreted neutral sterols from the living system; c) measuring isotopic content, isotopic pattern, rate of change of isotopic content, or isotopic pattern of the isotopically labeled cholesterol molecules, bile acids or excreted neutral sterols; and d) calculating the rate of incorporation or transfer of the isotopically labeled cholesterol molecule or isotopically labeled cholesterol-related molecule into the cholesterol molecules, bile acids or excreted neutral sterols to determine the molecular flux rate of the hepatic or excretory component of RCT in the living system.

In further embodiments, the sample may be a stool, urine or blood sample.

The label of the isotopically labeled cholesterol molecule or isotopically labeled cholesterol-related molecule may be ²H, ³H, ¹³C, ¹⁴C, or ¹⁸O.

The living system may be a mammal. Mammals include humans, hamsters, rabbits, non-human primates and rodents.

In an additional embodiment, the total amount of bile acids in the living system may be determined by: i) administering a known amount of isotopically labeled bile acid to the living system; ii) determining the isotopic content or rate of change in isotopic content of bile acid in said living system after a period of time; and iii) determining the amount of dilution of the isotope labeled bile acid to measure the total amount of bile acids in the living system.

The labeled bile acids may include cholic acid, chenodeoxycholic acid, deoxycholic acid or lithocholic acid.

In another embodiment, a means to measure the contribution of de novo cholesterol synthesis to bile acid is provided, including: i) administering an isotopically labeled cholesterol precursor to a living system wherein the precursor has a defined label concentration; ii) obtaining a biological sample from the living system wherein the sample includes labeled bile acid, excreted neutral sterol or blood cholesterol; iii) measuring the isotopic content or pattern of said labeled bile acid, excreted neutral sterol or blood cholesterol; and iv) comparing the isotopic content of the bile acids, neutral sterols or cholesterol to the label concentration of the stable isotope-labeled cholesterol precursor to determine the fraction of cholesterol, neutral sterol or bile acids that are derived from newly synthesized cholesterol to measure the contribution by de novo cholesterol synthesis to bile acid. In a further embodiment, the sample may be a stool sample and the total content of neutral sterols and bile acids excreted by a subject per unit time is measured by comparison to an internal standard detected in the stool that was administered orally to the subject. The internal standard may be sitostanol.

In a further embodiment, a method for determining the molecular flux rate of the plasma component of reverse cholesterol transport (RCT) in a living system is described. The method may include: a) administering a stable, isotopically labeled cholesterol molecule or a stable isotopically labeled cholesterol-related molecule to a living system; b) obtaining a sample from the living system wherein the sample includes an in vivo conversion product of the isotopically labeled cholesterol molecule or the isotopically labeled cholesterol-related molecule; c) measuring isotopic content, isotopic pattern, rate of change of isotopic content, or isotopic pattern of the in vivo conversion product; and d) calculating the rate of dilution of the isotopically labeled cholesterol molecule or the isotopically labeled cholesterol-related molecule to determine the molecular flux rate of the plasma component of reverse cholesterol transport in the living system.

In a further embodiment, a method for determining the rate of appearance of cholesterol in blood, in a living system is described. The method may include: a) administering ¹³C₂ labeled cholesterol in a lipid emulsion intravenously to a living system at an administration rate sufficient to result in an accumulation of detectable levels of labeled, free cholesterol in said living system; b) obtaining samples from the living system wherein the samples include the labeled, free cholesterol molecule; c) measuring isotopic content, isotopic pattern, rate of change of isotopic content, or isotopic pattern of the labeled, free cholesterol molecule; and d) calculating the rate of appearance of cholesterol in blood in the living system by comparing the isotopic content, isotopic pattern, rate of change of isotopic content, or isotopic pattern of the labeled, free cholesterol molecule to the rate of administration of the ¹³C₂ labeled cholesterol.

In one embodiment, the rate of appearance of cholesterol in blood in the living system is calculated by the plateau principle, by isotope dilution, by establishing the existence of isotopic plateau, by inferring the isotopic plateau value or by extrapolating the isotopic plateau value.

A method for calculating the global rate of RCT the flux of cholesterol in a living system is also described. The method may include: a) measuring the rate of appearance of cholesterol in blood, by: i) administering ¹³C₂ labeled cholesterol in a lipid emulsion intravenously to a living system at an administration rate sufficient to result in an accumulation of detectable levels of labeled, free cholesterol in the living system; ii) obtaining samples from the living system wherein the samples include a labeled, free cholesterol molecule; iii) measuring isotopic content, isotopic pattern, rate of change of isotopic content, or isotopic pattern of the labeled, free cholesterol molecule; and iv) calculating the rate of appearance of cholesterol in blood in the living system by comparing the isotopic content, isotopic pattern, rate of change of isotopic content, or isotopic pattern of the labeled, free cholesterol molecule to the rate of administration of the ¹³C₂ labeled cholesterol; b) measuring the percentage recovery of the hepatic or excretory arm of reverse cholesterol transport (RCT) by: i) administering an isotopically labeled cholesterol molecule or isotopically labeled cholesterol-related molecule to the living system; ii) obtaining a sample from the living system wherein the sample includes one or more isotopically labeled cholesterol molecules, bile acids or excreted neutral sterols from the living system; iii) measuring isotopic content, isotopic pattern, rate of change of isotopic content, or isotopic pattern of the isotopically labeled cholesterol molecules, bile acids or excreted neutral sterols; and iv) calculating the rate of incorporation or transfer of the isotopically labeled cholesterol molecules or isotopically labeled cholesterol-related molecule into the cholesterol molecules, bile acids or excreted neutral sterols to determine the percentage recovery of the hepatic or excretory component of RCT in the living system; c) calculating the rate of global RCT the flux of cholesterol in the living system by multiplying the rate of appearance of cholesterol in blood from a) iii) by the percentage recovery of the hepatic or excretory arm of RCT from b) iv).

In a further embodiment, a method of assessing the effect of a candidate agent and/or dietary modification on the risk for and rate of development of atherosclerosis in a living system is described. The method may include: a) calculating the rate of global RCT the flux of cholesterol in the living system; b) administering said candidate agent to the living system and/or modifying the diet of the living system; c) calculating the global RCT the flux of cholesterol in the living system a second time; and d) comparing the difference between the cholesterol rate fluxes of steps b) and c) to assess the effect of the candidate agent and/or the dietary modification on atherosclerosis.

The method may also include: a) determining the molecular flux rate of the hepatic or excretory component of reverse cholesterol transport (RCT) in a living system; b) administering the candidate agent to the living system and/or modifying the diet of the living system; c) determining the molecular flux rate of the hepatic or excretory component of reverse cholesterol transport (RCT) in the living system a second time; and d) comparing the difference between the molecular rate fluxes of steps b) and c) to assess the effect of the candidate agent and/or the dietary modification on atherosclerosis.

The method may further include: a) determining the molecular flux rate of the plasma component of reverse cholesterol transport (RCT) in a living system; b) administering the candidate agent to the living system and/or modifying the diet of the living system; c) determining the molecular flux rate of the plasma component of reverse cholesterol transport (RCT) in a living system; and d) comparing the difference between the molecular rate fluxes of steps b) and c) to assess the effect of the candidate agent and/or the dietary modification on atherosclerosis.

### BRIEF DESCRIPTION OF THE DRAWINGS

FIG. 1 illustrates the RCT pathway and whole-body pools of cholesterol. Abbreviations; “RBC”, red blood cell. The existence of a large, slow-turnover pool of cholesterol in peripheral tissues and a rapid turnover pool of cholesterol that comprises plasma, RBC and hepatic free cholesterol, is shown. Labeled cholesterol administered into the rapid turnover pool can exchange with tissues or be excreted in the form of fecal sterols.

FIG. 2 illustrates the recognized molecular elements of the RCT pathway (after A. Tall, Journal of Clinical Investigation, 2001).

FIG. 3 illustrates two components or arms of the RCT pathway, efflux/mobilization and excretion.

FIG. 4A illustrates the plateau principle and the model for measuring flux rate or rate of appearance (Ra) of a molecule by isotope dilution. FIG. 4B illustrates the application of the plateau principle for measuring Ra of cholesterol and the protocol for measuring Ra cholesterol.

FIG. 5 illustrates ¹³C APE versus hours of ¹³C₂-cholesterol infusion for three human subject. Curve fits and fit parameters for an exponential approach to plateau are included on each graph.

FIG. 6A illustrates representative values of cholesterol efflux/mobilization rate, or Ra cholesterol, measured in human subjects. FIG. 6B illustrates within-subject reproducibility for repeat measurements of Ra cholesterol.

FIG. 7 illustrates the efflux rates of cholesterol in rats given different diets. Changes in plasma cholesterol Ra, increased with cholesterol/cholic acid feeding, and persists 4 days after returning to normal diet.

FIG. 8A illustrates the protocol for measuring the excretion efficiency of administered labeled cholesterol in fecal sterols. FIG. 8B illustrates the effect of treatment for 7 days of a rat with an LXR agonist on the excretion efficiency of administered labeled cholesterol.

FIG. 9A illustrates the need to correct excretion efficiency of administered labeled cholesterol for efflux/influx across tissues (Ra cholesterol), due to artifactual reduction in excretion efficiency if Ra is increased in an individual. FIG. 9B illustrates the basis of the calculation of the Global RCT parameter, representing cholesterol flux from tissues through blood into stool. FIG. 9C illustrates the Global RCT process measured, comprising flux from tissue to blood (efflux/mobilization) and from blood to stool (excretion).

FIG. 10A illustrates the components and calculation of the “global parameter” of RCT into neutral sterols in rats fed the LXR agonist (TO-901317). Rats were fed the LXR agonist for 7 days and RCT fluxes were determined. A) Ra cholesterol. B) Excretion of plasma cholesterol into bile acids. C) Product of the two components (significant increase observed). FIG. 10B illustrates the relation between LXR-induced gene expression and global RCT flux.

FIG. 11A illustrates the components and calculation of the “global parameter” of RCT into neutral sterols in rats fed cholestyramine. Rats were fed the bile acid binding agent cholestyramine for 7 days and RCT fluxes were determined. A) Ra cholesterol. B) Excretion of plasma cholesterol into neutral sterols. C) Product of the two components (no significant effect observed). FIG. 11B illustrates the components and calculation of the “global parameter” of RCT in bile acids in rats fed cholestyramine. Rats were fed bile acid binding agent (cholestyramine) for 7 days and RCT fluxes were determined. A) Ra cholesterol. B) Excretion of plasma cholesterol into bile acids. C) Product of the two components (significant increase observed).

FIG. 12 illustrates the components and calculation of the “global parameter” of RCT on neutral sterols in rats fed ezetimibe. Rats fed ezetimibe for 7 days and then RCT fluxes were determined. A) Ra cholesterol. B) Excretion of plasma cholesterol into bile acids. C) Product of the two components (significant increase observed).

FIG. 13A illustrates the parameters of RCT into fecal neutral sterols measured in human subjects, including Global RCT and the two component arms of RCT. Relationship to low or high concentrations of HDL-cholesterol in the subjects is shown. FIG. 13B illustrates the parameters of RCT into fecal bile acids measured in human subjects, including Global RCT and the two component arms of RCT. Relationship to low or high concentrations of HDL-cholesterol in the subjects is shown. FIG. 13C illustrates the average values for Global RCT into fecal neutral sterols and bile acids in human subjects.

FIG. 14 illustrates the de novo cholesterol synthesis rates measured in rats and the effects of drugs and diet on de novo cholesterol synthesis rates.

FIG. 15 illustrates a non-limiting list of isotope-labeled cholesterol derivatives (including cholesterol) and cholesterol-esters, detailing their structure and where label might attach to each molecule.

FIG. 16 illustrates a non-limiting list of isotope-labeled bile acids and bile acid metabolites, detailing their structure and where label might attach to each molecule.

FIG. 17A illustrates the protocol for measuring efflux/mobilization (Ra) in humans. FIG. 17B illustrates the protocol for measuring cholesterol excretion efficiency in humans.

### DETAILED DESCRIPTION OF THE INVENTION

The present invention provides a means for quantitatively measuring RCT in vivo using isotopic and mass spectrometric techniques.

General Techniques

The practice of the present invention will generally utilize, unless otherwise indicated, conventional techniques of molecular biology, microbiology, cell biology, biochemistry, and immunology, which are within the skill of the art. Such techniques are fully explained in the literature, for example, in Cell Biology: A Laboratory Notebook (J. E. Cellis, ed., 1998); Current Protocols in Molecular Biology (F. M. Ausubel et al., eds, 1987); Short Protocols in Molecular Biology (Wiley and Sons, 1999); Mass Isotopomer Distribution Analysis: A Technique for Measuring Biosynthesis and Turnover of Polymers (Hellerstein et al., Am J Physiol 263 (Endocrinol Metab 26):E988-E1001 (1992)); and Mass Isotopomer Distribution Analysis at Eight Years: Theoretical, Analytic, and Experimental Considerations (Hellerstein et al., Am J Physiol 276 (Endocrinol Metab 39):E1146-1170 (1999)). Furthermore, procedures employing commercially available assay kits and reagents will typically be used according to manufacturer-defined protocols unless otherwise noted.

Practice of the invention will additionally utilize, unless otherwise indicated, conventional techniques of chemistry and analytic chemistry, which are within the skill of the art. Such techniques are fully explained in the literature, for example, in Fundamentals of Analytical Chemistry (D. Skoog, D West, F Holler, S Crouch, auth, 2003); Analytical Chemistry (S. Higson, auth, 2004); Advanced Instrumental Methods of Chemical Analysis (J. Churacek, ed, 1994); and Advanced mass spectrometry: Applications in organic and analytical chemistry (U. Schlunegger).

Practice of the invention will additionally utilize, unless otherwise indicated, conventional techniques of pre-clinical and clinical research, which are within the skill of the art. Such techniques are fully explained in the literature.

Definitions

By “cholesterol or cholesterol-related molecules or cholesterol-related complexes” is meant molecules and complexes that are part of cholesterol metabolism and transport. These include cholesterol and cholesterol metabolites or precursors, or cholesterol precursors (e.g., water, acetyl-CoA) and complexes of cholesterol and cholesterol metabolites or precursors with carriers such as lipoproteins (e.g., high density lipoprotein). The following non-limiting list includes examples of “cholesterol or cholesterol-related molecules or complexes”: cholesterol derived from chylomicrons, triglyceride rich lipoproteins (TGRL), high density lipoprotein (HDL), intermediate density lipoprotein (IDL), low density lipoprotein (LDL), or very low density lipoprotein (VLDL); cholesterol ester derived from chylomicrons, TGRL, HDL, IDL, LDL, or VLDL; bile acids from blood, stool, bile, urine, or any other biological sample; neutral sterols from blood, stool, bile, urine or any other biological sample; chenodeoxycholates, cholates, deoxycholates, lithocholates, ursodeoxycholates, phospholipids, or bilirubin; the metabolites of bile acids generated by gastrointestinal microbes; metabolites of neutral sterols generated by gastrointestinal microbes (e.g., coprostanol, coprostanone, cholestanol, cholestanone and epicoprostanol), and others.

By “samples containing molecule or complex” is meant any sample containing the indicated molecule or complex. Any concentration of the indicated molecule or complex is considered. The isotopic content or isotopic pattern of the indicated molecule or complex may vary. It may be zero. The indicated molecule or complex may only exist in the sample as a portion of another complex.

By “complex” or “complexes” is meant any macromolecular assembly made up of one or more molecules. The molecules may be lipids, small molecules, proteins, lipoproteins, or others. An example of a complex is HDL, which contains a variety of molecules, including smaller molecules (including, cholesterol and cholesterol ester) and larger molecules (e.g., lipoproteins). Another example of a complex is LDL.

By “bile acid” is meant any bile acid or bile acid metabolic product (i.e., metabolite) found in the stool, blood, or other biological sample or component. Examples of bile acids include cholic acid, deoxycholic acid, chenodeoxycholic acid, and any other bile acid or bile acid metabolite.

By “bile” is meant the secretions from the gall bladder into the gastrointestinal lumen. A partial list of bile components includes bile acids, neutral sterols, chenodeoxycholates, cholates, deoxycholates, lithocholates, cholesterol, ursodeoxycholates, phospholipids, or bilirubin. In the gastrointestinal lumen, some bile components are metabolized by resident microbes to bile component derivatives or metabolites (e.g., coprostanol, coprostanone, cholestanol, cholestanone and epicoprostanol are metabolites of bile cholesterol). For the purposes of the present invention, these derivatives are considered components of bile as well.

By “activity” is meant a measure of RCT or a component of RCT that can be determined in a subject using the methods of the present invention. Activity may be represented by a rate (e.g., a quantity per unit time, or a quantity of cholesterol or cholesterol-related molecule or cholesterol-related complex converted or transported per unit time), a mass (e.g., grams, or grams of cholesterol or cholesterol-related molecule or cholesterol-related complex), a fraction or percent (e.g., fraction of bile acids derived from RCT, or percent of neutral sterols derived from RCT), or any other representation of the data acquired during the practice of the methods disclosed herein. Activity may be what is compared between different subjects, or what is compared between the same subject before and after administration of a candidate therapy, or what is compared to historical data. In some circumstances, the same data may be represented as a number of different types of activity. Data may be combined with historical data, baseline data, or new data in order to calculate a new type of activity (e.g., the fractional contribution of RCT to bile acids can be combined with the bile acid pool size to determine the mass of cholesterol or cholesterol-related molecule or cholesterol-related complex converted to bile acids).

By “historical data” is meant any data existing prior to the commencement of the experiment.

By “candidate therapy” is meant any process by which a disease may be treated that can be screened for effectiveness as outlined herein. Candidate therapies may include behavioral, exercise, or dietary regimens. Candidate therapies may also include treatments with a medical device, or the implantation of a medical device. Candidate therapies may also include therapy with any “candidate agent” or “candidate drug” (see, infra).

Candidate therapies may include combinations of candidate therapies. Such a combination may be two different candidate agents. A combination may also be a candidate agent and a dietary regimen. A combination may also be an exercise regimen and a dietary regimen. A combination may also be an exercise regimen and a dietary regimen and a candidate agent. A combination may also be a combination of candidate agents or a combination of candidate agents coupled with another candidate therapy such as exercise or a dietary regimen or both. A combination is therefore more than one candidate therapy administered to the same subject.

Candidate therapies may already be approved for use in humans by an appropriate regulatory agency (e.g., the U.S. Food and Drug Administration or a foreign equivalent). Candidate therapies may already be approved for use in humans for the treatment or prevention of atherogenesis, arteriosclerosis, atherosclerosis, or other cholesterol-related diseases.

By “candidate agent” or “candidate drug” is meant any compound, molecule, polymer, macromolecule or molecular complex (e.g., proteins including biotherapeutics such as antibodies and enzymes, small organic molecules including known drugs and drug candidates, other types of small molecules, polysaccharides, fatty acids, vaccines, nucleic acids, etc) that can be screened for activity as outlined herein. Candidate agents are evaluated in the present invention for discovering potential therapeutic agents that affect cholesterol metabolism and transport.

Candidate agents encompass numerous chemical classes. In one embodiment, the candidate agent is an organic molecule, such as small organic compounds, which generally have a molecular weight of between 100 and about 2,500 daltons. Preferred are small organic compounds having a molecular weight of more than 100 and less than about 2,000 daltons, more preferably less than about 1500 daltons, still more preferably less than about 1000 daltons, and yet still more preferably less than 500 daltons. Candidate agents comprise functional groups necessary for structural interaction with proteins or other host molecules, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents include “known drugs” or “known drug agents” or “already-approved drugs”, such terms refer to agents that have been approved for therapeutic use as drugs in human beings or animals in the United States or other jurisdictions. Known drugs also include, but are not limited to, any chemical compound or composition disclosed in, for example, the 13th Edition of The Merck Index (a U.S. publication, Whitehouse Station, N.J., USA), incorporated herein by reference in its entirety.

Candidate agents are obtained from a wide variety of sources including libraries of synthetic or natural compounds. For example, numerous means are available that are well known in the art for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs and thereby rendering them distinct candidate agents.

The candidate agents may be proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and norleucine are considered amino acids for the purposes of the invention. “Amino acid” also includes imino acid residues such as proline and hydroxyproline. The side chains may be in either the (R) or the (S) configuration. If non-naturally occurring side chains are used, non-amino acid substituents may be used, for example to prevent or retard in vivo degradations. Peptide inhibitors of enzymes find particular use.

The candidate agents may be naturally occurring proteins or fragments of naturally occurring proteins. Thus, for example, cellular extracts containing proteins, or random or directed digests of proteinaceous cellular extracts, may be used. In this way libraries of prokaryotic and eukaryotic proteins may be made for screening in the systems described herein. Particularly preferred in this embodiment are libraries of bacterial, fungal, viral, and mammalian proteins, with the latter being preferred, and human proteins being especially preferred.

The candidate agents may be antibodies, a class of proteins. The term “antibody” includes full-length as well antibody fragments, as are known in the art, including Fab Fab2, single chain antibodies (Fv for example), chimeric antibodies, humanized and human antibodies, etc., either produced by the modification of whole antibodies or those synthesized de novo using recombinant DNA technologies, and derivatives thereof.

The candidate agents may be nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook, and peptide nucleic acids. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xathanine, hypoxathanine, isocytosine, isoguanine, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine, etc.

As described above generally for proteins, nucleic acid candidate agents may be naturally occurring nucleic acids, random and/or synthetic nucleic acids. For example, digests of prokaryotic or eukaryotic genomes may be used as is outlined above for proteins. In addition, RNA interference sequences (RNAi's) are included herein.

By “subject” is meant the living subject of the experiment or procedure or process being described. All subjects are living systems. In one embodiment, a subject may be a human. In another embodiment, a subject may be a rabbit or a rodent or a non-human primate. Additionally, the term “subject” encompasses any other living system.

By “living system” is meant herein any living entity including a cell, cell line, tissue, organ or organism. Examples of organisms include any animal, preferably a vertebrate, more preferably a mammal, most preferably a human. Examples of mammals include nonhuman primates, farm animals, pet animals (e.g., cats and dogs), and research animals (e.g., mice, rats, and humans).

A “biological sample” encompasses any sample obtained from a living system or subject. The definition encompasses blood, tissue, and other samples of biological origin that can be collected from a living system or subject. Preferably, biological samples are obtained through sampling by minimally invasive or non-invasive approaches (e.g., urine collection, stool collection, blood drawing, needle aspiration, and other procedures involving minimal risk, discomfort or effort). Biological samples are often liquid (sometimes referred to as a “biological fluid”). Liquid biological samples include, but are not limited to, urine, blood, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, and others. Biological samples include samples that have been manipulated in any way after their procurement, such as by treatment with reagents, solubilization, or enrichment for certain components, such as proteins or polynucleotides. The term “biological sample” also encompasses a clinical sample such as serum, plasma, other biological fluid, or tissue samples, and also includes cells in culture, cell supernatants and cell lysates.

“Isotopic content or isotopic pattern or rate of change of isotopic content or isotopic pattern” refers to the isotopic content, isotopic pattern, rate of change of isotopic content, rate of change of isotopic pattern, or other measurements of the isotopic disposition of a molecule. Isotopic content or isotopic pattern or rate of change of isotopic content or rate of change of isotopic pattern refers to the pattern or content or distribution of isotopes in a molecule or population of molecules. This term includes a broad range of meanings and molecular properties. Isotopic content or isotopic pattern may include the change in isotopic content over time (e.g., the isotopic content as a function of time). In one embodiment, isotopic content or isotopic pattern indicates the mole percent of a given isotope in a sample. In another embodiment, isotopic content or isotopic pattern indicates the relative amounts of one or more mass isotopomers. In another embodiment, isotopic content or isotopic pattern may refer to the entire distribution of mass isotopomers, which for a large molecule may number in the hundreds. Isotopic content or isotopic pattern may also indicate the relative amount of a single highly labeled species (e.g., a precursor with four deuteriums on it). Isotopic content or isotopic pattern can refer to the molar percent excess of a single isotopomer, or it can refer to the atom percent excess of a particular isotope. Isotopic content or isotopic pattern includes what is measured by the mass spectrometric analyses carried out during the practice of the present methods described herein. These analyses may include the determination of the molar percent excess (MPE) of a particular mass isotopomer (e.g., the MPE of the M₁ isotopomer of cholesterol, or the MPE of the M₄ isotopomer of cholic acid, or the MPE of ²H₂O in blood). These analyses may include the determination of the atomic percent excess (APE) of a particular atomic isotope (e.g., the APE of ¹³C in cholesterol).

By “molecule of interest” is meant a cholesterol or cholesterol-related molecule or cholesterol-related complex chosen for purification or analysis during the practice of the methods described herein. Such a molecule of interest is the isotopically-labeled product found or generated in the subject while practicing the methods described herein. For instance, measurement of the hepatic or excretory component of RCT may be based on the conversion of ¹³C₂-labeled cholesterol to ¹³C₂-labeled cholic acid in the liver of a subject. In this case, cholic acid is the molecule of interest, and it is analyzed for its ¹³C isotopic content or isotopic pattern. Similarly, measurement of the plasma component of RCT may be based on the conversion of ¹⁴C₂-labeled cholesterol in HDL to ¹⁴C₂-labeled cholesterol-ester in VLDL. In this case, cholesterol ester derived from VLDL is the molecule of interest, and it is analyzed for its ¹⁴C isotopic content or isotopic pattern. The molecule of interest may be the same as the isotopically-labeled molecule (the cholesterol or cholesterol-related molecule or cholesterol-related complex or cholesterol precursor) administered to the subject, or it may be a different isotopically-labeled molecule, for example, one generated by a metabolic event, either by the subject or a microorganism within the subject.

Molecules of interest may include cholesterol or cholesterol ester. Molecules of interest may also include cholesterol derived from chylomicrons, TGRL, HDL, IDL, LDL, or VLDL. Molecules of interest may also include cholesterol ester derived from chylomicrons, TGRL, HDL, IDL, LDL, or VLDL. Molecules of interest may also include bile acids from blood, stool, bile, urine, or any other biological sample. Molecules of interest may also include neutral sterols from blood, stool, bile, urine or any other biological sample. Molecules of interest may also include chenodeoxycholates, cholates, deoxycholates, lithocholates, ursodeoxycholates, phospholipids, or bilirubin. Molecules of interest may also include the metabolites of bile acids generated by gastrointestinal microbes. Molecules of interest may also include metabolites of neutral sterols (e.g., coprostanol, coprostanone, cholestanol, cholestanone and epicoprostanol) generated by gastrointestinal microbes. Such microbial metabolites may be found in the stool, or they may be reabsorbed into the subject and may appear in other biological samples.

Isotope labeled molecules of interest, which are a fraction of the total pool of molecules of interest in a subject or in a biological sample, are products generated by practicing the methods of the present invention.

“Monoisotopic mass” refers to the exact mass of the molecular species that contains all ¹H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as m0 and the masses of other mass isotopomers are identified by their mass differences from m0 (m1, m2, etc.).

“Mass isotopomer” or “isotopomer” refers to a family of isotopic isomers that is grouped on the basis of nominal mass rather than isotopic composition. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). In operational terms, a mass isotopomer is a family of isotopologues that are not resolved by a mass spectrometer. For quadrupole mass spectrometers, this typically means that mass isotopomers are families of isotopologues that share a nominal mass. Thus, the isotopologues CH₃NH₂ and CH₃NHD differ in nominal mass and are distinguished as being different mass isotopomers, but the isotopologues CH₃NHD, CH₂DNH₂, ¹³CH₃NH₂, and CH₃¹⁵NH₂ are all of the same nominal mass and hence are the same mass isotopomers. Each mass isotopomer is therefore typically composed of more than one isotopologue and has more than one exact mass. The distinction between isotopologues and mass isotopomers is useful in practice because all individual isotopologues are not resolved using quadrupole mass spectrometers and may not be resolved even using mass spectrometers that produce higher mass resolution, so that calculations from mass spectrometric data must be performed on the abundances of mass isotopomers rather than isotopologues. The mass isotopomer lowest in mass is represented as M₀; for most organic molecules, this is the species containing all ¹²C, ¹H, ¹⁶O, ¹⁴N, etc. Other mass isotopomers are distinguished by their mass differences from M₀ (M1, M2, etc.). For a given mass isotopomer, the location or position of isotopes within the molecule is not specified and may vary (i.e., “positional isotopomers” are not distinguished).

By “isotope-labeled” is meant labeled with atoms with the same number of protons and hence of the same element but with different numbers of neutrons (e.g., ¹H vs. ²H). Isotope-labeled molecules are labeled with any possible isotope. Isotopes may be stable isotopes (e.g., ²H, ¹³C) or they may be radioisotopes (e.g., ³H, ¹⁴C).

“Isotope-labeled substrate” includes any isotope-labeled precursor molecule that is able to be incorporated into a molecule of interest in a living system or subject. Examples of isotope labeled substrates include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃.

“Purifying” refers to methods of removing one or more components of a mixture of other similar compounds. For example, “purifying a protein or peptide” refers to removing a protein or peptide from one or more proteins or peptides in a mixture of proteins or peptides.

“Isolating” refers to separating one compound from a mixture of compounds. For example, “isolating a protein or peptide” refers to separating one specific protein or peptide from all other proteins or peptides in a mixture of one or more proteins or peptides.

“Precursor molecule” refers to the metabolic precursors used during synthesis of specific molecules. Examples of precursor molecules include acetyl CoA, ribonucleic acids, deoxyribonucleic acids, amino acids, glucose, water, and others.

“Labeled water” as used herein refers to water that contains isotopes. Examples of labeled water include ²H₂0, ³H₂0, and H₂¹⁸0. As used herein, the term “isotopically labeled water” is used interchangeably with “labeled water.”

“Molecular flux rates” refers to the rate of synthesis and/or breakdown of molecules within a cell, tissue, or organism. “Molecular flux rates” also refers to a molecule's input into or removal from a pool of molecules, and is therefore synonymous with the flow into and out of said pool of molecules.

Methods for Measuring Cholesterol Transport and Metabolism

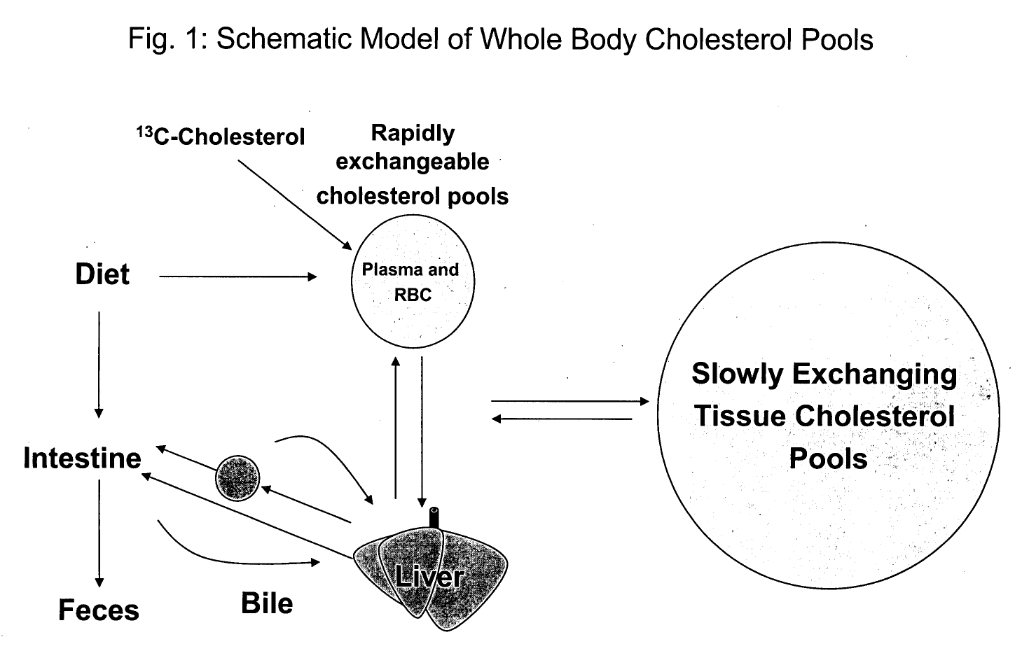

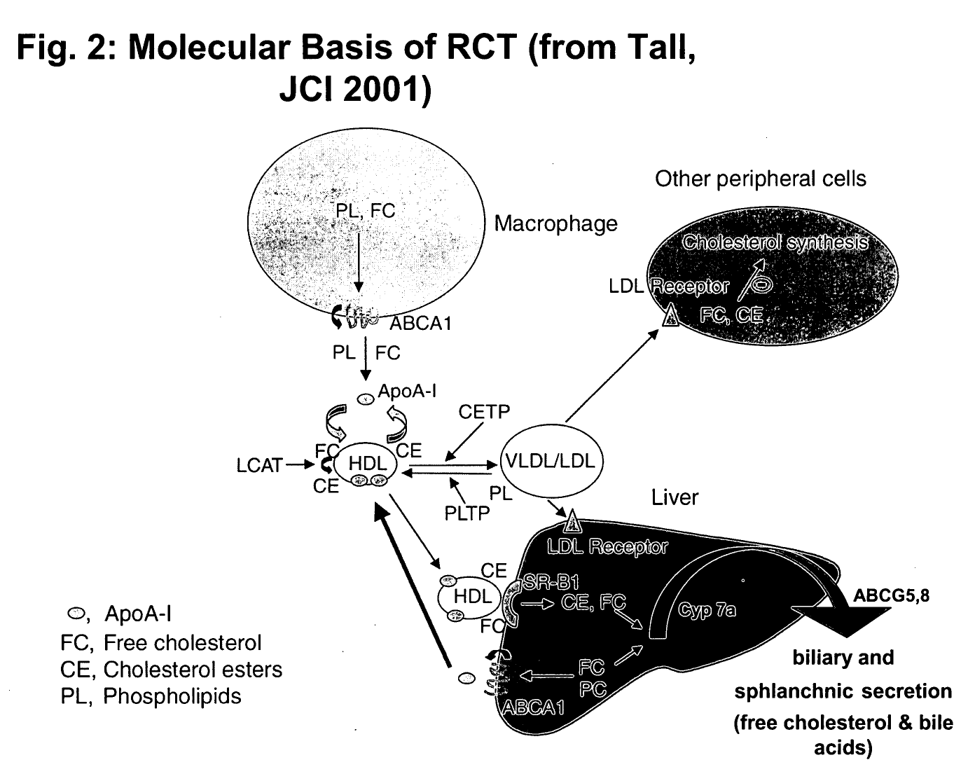

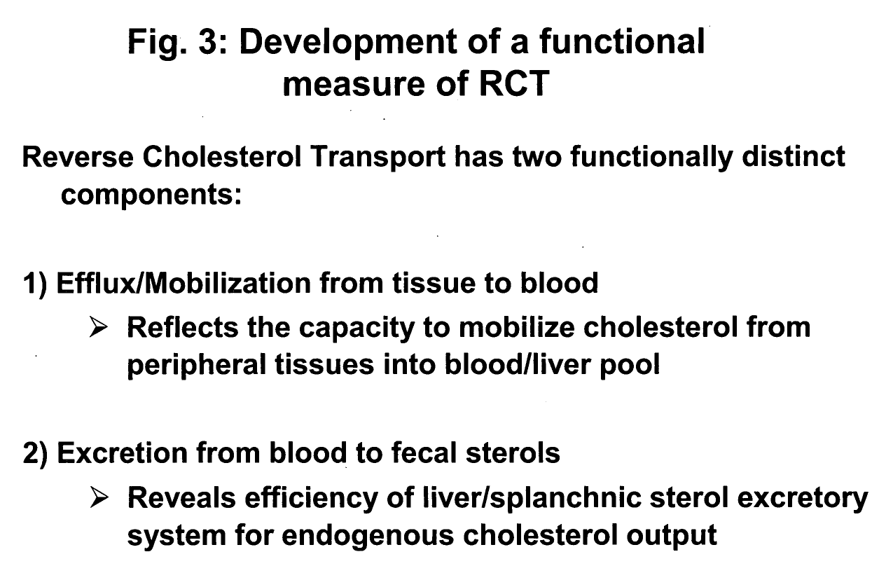

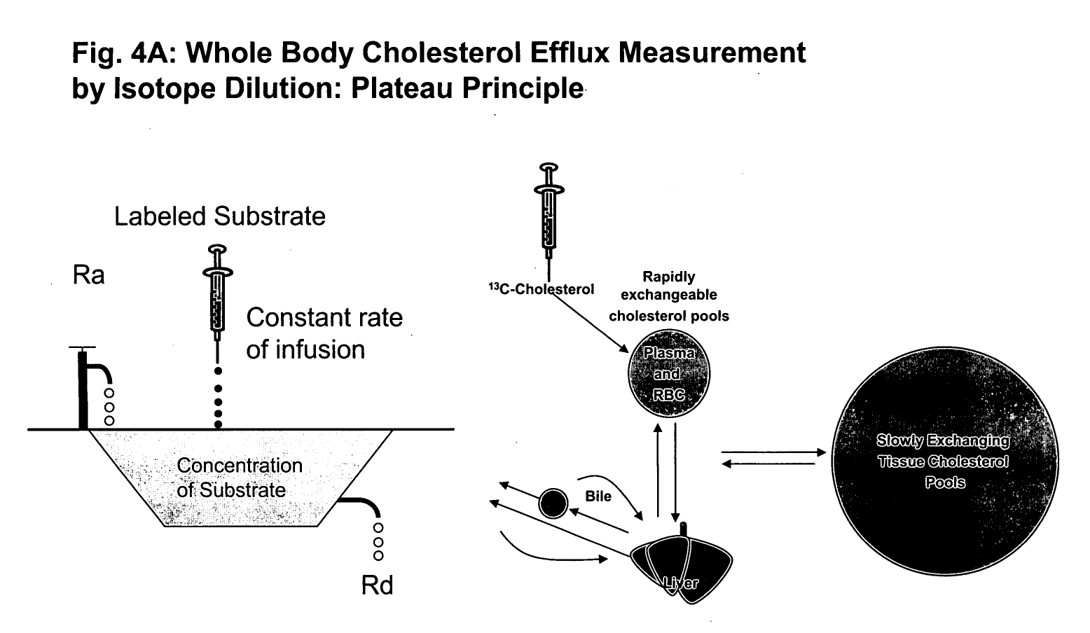

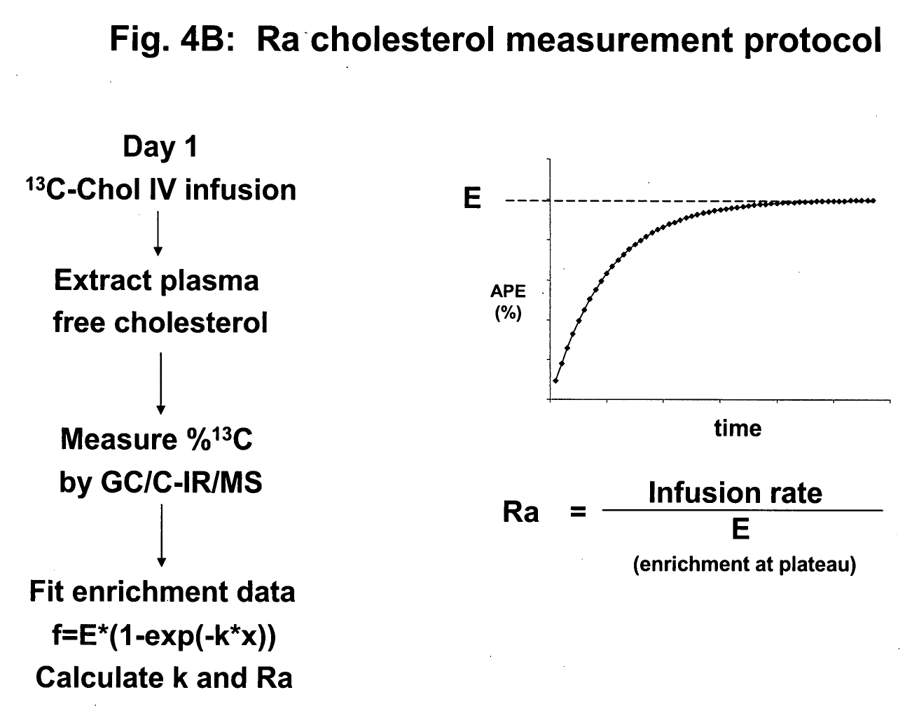

Reverse cholesterol transport (RCT) is a biological pathway through which cholesterol is mobilized and transported out of the body (FIG. 1). There are three components of RCT: (1) efflux of cholesterol from tissues, particularly extrahepatic tissues, into the bloodstream (the efflux component, FIG. 2); (2) the transport and distribution of cholesterol within the plasma compartment (the plasma component, FIG. 3) and; (3) and excretion into the feces via the liver or intestine (the hepatic or excretory component, FIG. 4). Cholesterol is incorporated into bile secretions either as bile acids or free cholesterol (sterols), which are then secreted into the intestinal lumen, and a portion of which leaves the body in the stool. Sterols may also be released directly from the intestine into the gut lumen, for subsequent excretion of a portion into the feces. These pathways represent the only significant mechanisms by which cholesterol can be removed from the body. From the functional point-of-view, components # 1 and #3 (i.e., the efflux component and the hepatic or excretory component) are the key steps, as these represent the exit of cholesterol from cells and from the body, respectively. As mentioned above, because of the well-established role of cholesterol in atherogenesis, atherosclerosis and other cholesterol-related diseases and diseases of the blood vessels, RCT is considered a key anti-atherogenic and anti-atherosclerotic process, and is generally believed to be the explanation for anti-atherogenic and anti-atherosclerotic properties as well as the clinical correlation with reduced cardiovascular risk of the high density lipoprotein (HDL) fraction of plasma.

However, HDL levels are now recognized to reflect only one component of the molecular pathway of RCT, and do not necessarily reflect the true flow of cholesterol through the RCT pathway. The molecular details of the RCT pathway have come into increasing focus in the past several years. One important implication of these recent advances in molecular understanding is the recognition that plasma HDLc (HDL-cholesterol) levels in isolation may or may not reflect true flux through the pathway, depending on the underlying mechanism responsible for the change in HDLc. For example, if the plasma concentration of HDLc in an individual represents flux from tissues through ABC(A)-1 (the ATP-binding cassette transporter) into plasma apoAI-containing particles, as in mutant ABC(A)-I heterozygotes, then HDLc is a useful marker. However, if HDLc in another individual accumulates because of inhibition of delivery of HDLc to its acceptors (e.g., due to reduced cholesterol ester transfer protein activity or reduced hepatic scavenger receptor-BI [SR-BI] activity), then HDLc levels will not reflect RCT. The situation can be particularly complex when considering the impact on RCT of interventions that alter the production and fate of apoB-containing particles, such as the statins. Because apoB particles are capable of carrying cholesterol forward (i.e., to the tissues) as well as in reverse (i.e., back to the liver), the actual fate of apoB particles in an individual may contribute to the efficiency of RCT at any plasma HDL level. The possibility of an increase in the already-existing dissociation between HDLc concentrations and RCT is thereby raised in the settings of effective statin therapy or other interventions that promote the return of VLDL and LDL particles to the liver, or indeed any therapy directed toward the modification of cholesterol or lipid metabolism (e.g., statin therapy, fibrate therapy and others).

The difference between RCT activity and HDLc levels illustrates the basis of the present disclosure. Measuring a biochemical process such as RCT is not the same as measuring the concentration of biochemical molecules. Measuring the concentration of HDLc is a technically simple task, but it is not, by itself, always informative. The process of interest is not HDLc concentration in plasma—the process of interest is the removal of cholesterol from the body (i.e., RCT). As such, measurement of HDLc is only a proxy to the real process. Several examples of this distinction are known in the art for animal models as well as humans. Genetic deletion of the SR-B1 receptor in mice prone to atherosclerosis impairs the RCT process by reducing the uptake of cholesterol from HDL (FIG. 1), thereby markedly increasing plasma HDL-cholesterol levels. Nevertheless, these mice exhibit worse atherosclerosis, not better, despite the higher HDL levels. This dissociation between HDL cholesterol levels and cardioprotection reflects the primacy of flux over concentrations of HDL. Similarly, genetic over-expression of the protein ABC-A1 in mice prone to atherosclerosis results in lower HDL cholesterol levels but protection against atherosclerosis. Indeed, the higher the HDL levels, the worse the atherosclerosis that was observed, opposite to the usual relationship between HDL and vascular risk. Moreover, humans with the mutation ApoA1-Milano have lower HDL-cholesterol levels but markedly reduced cardiovascular risk, presumably reflecting the superior capacity of ApoA1-Milano to carry cholesterol through the RCT pathway without accumulation in the bloodstream.

Thus, a method which could actually measure the amount of cholesterol that is transported through the RCT pathway from tissues through blood and out of the body would be a direct measurement of the process in question. The present disclosure describes measuring the amount (absolute, relative, or fractional) of cholesterol or cholesterol-related molecules or cholesterol-related complexes which are synthesized, transported, modified, metabolized, secreted, or otherwise moved through a living system or subject, with an emphasis on the RCT pathway. Methods such as these are often grouped under the heading of “kinetic” measurements, because they sometimes, though not necessarily, involve a timed experiment, or return data in the form of a rate (i.e., a quantity per unit time). In the present disclosure, isotope labels are used to track the movement of cholesterol or cholesterol-related molecules or cholesterol-related complexes through the different components of the RCT pathway. Isotope-labeled molecules are chemically identical to and biochemically indistinguishable from non-labeled ones, but they have a different mass, a property that can be measured by a variety of mass spectrometric or other methods such as laser spectrophotometry or laser spectroscopy. By tracking the appearance, dilution, enrichment, or disappearance of isotope-labeled molecules, the transport or metabolism of cholesterol or cholesterol-related molecules or cholesterol-related complexes can thereby be measured.

Due to the growing global impact of cholesterol-related disease, and due to the complexities of cholesterol metabolism and its study, an in vivo method for measuring the rate of RCT is needed and would have great utility for medical care and drug discovery and development. The identification, selection, evaluation and development (e.g., clinical or pre-clinical dose finding and optimization of dosages, measurement of efficacy) of candidate therapies, the diagnosis of cholesterol-related disease, the management of subjects, including evaluation of disease progression or response to therapy, and the design and testing of medical devices or tools for use in cholesterol-related disease management, diagnosis or treatment are all processes which would benefit from the practice of the methods of the present invention. Evaluating subjects prior to enrollment in clinical trials is one beneficial use of the present invention. Evaluating subjects in order to predict whether or not they will respond to a candidate therapy is another beneficial use of the present invention. The invention has further uses directed toward the identification and study of genetic factors that alter the risk for cholesterol-related disease, and toward the development of disease criteria (i.e., a combination of risk factors that indicate a disease or pre-disease state) that can be used to classify subjects and recommend treatment.

The present disclosure provides methods for measuring cholesterol transport and metabolism in vivo. In one embodiment, the disclosure is more specifically directed toward the measurement of RCT, the mechanism by which cholesterol leaves cells and the body. The term “reverse cholesterol transport” or “RCT” is used to describe the entire process by which cholesterol moves from cells into the bloodstream and from the bloodstream out of the body. The RCT process may include the transient or permanent metabolism or modification of cholesterol, and as such does not deal exclusively with the transport of cholesterol, but rather with a variety of cholesterol precursors and derived products. Furthermore, during RCT, cholesterol and its various metabolites are associated with, and are transferred between, a broad range of carrier molecules or complexes, such as HDL subclasses and very-low-density-lipoproteins (VLDL). The RCT process is not necessarily unidirectional, as some components of RCT (e.g., bile acids) are secreted and reabsorbed, and others are transported or modified reversibly and exist in equilibrium (i.e., a state wherein there is free transport in both directions of the process in question). In the context of the present disclosure, the term “RCT” is used to describe the process by which cholesterol and cholesterol metabolites or cholesterol-related molecules are eventually removed from a living organism (i.e., a living system or a subject). The RCT process may be characterized as a three-part process, with an “efflux component”, a “plasma component” and a “hepatic or excretory component” (FIG. 1). The present disclosure is directed toward the measurement of RCT as a whole, although any of the three components can be independently measured without the need to measure or evaluate the other components.

The methods are generally carried out in mammalian subjects, including humans. Mammals include, but are not limited to, primates, farm animals, sport animals, pets such as cats and dogs, guinea pigs, rabbits, hamsters, mice, rats, humans and the like.

I. Determining the Rate of the Efflux Component of RCT in a Subject.

In one aspect, the efflux component of RCT may be determined in a subject. One or more isotopically-labeled cholesterol or cholesterol-related molecules or cholesterol-related complexes are administered to the subject. After a period of time, one or more samples, such as plasma samples, containing cholesterol or cholesterol-related molecules or cholesterol-related complexes are obtained from the subject, and the isotopic content or isotopic pattern of the molecule or complex is measured. The amount of dilution of the administered label by the efflux of endogenous cholesterol or cholesterol-related molecules or cholesterol-related complexes from tissues is calculated from this data, directly reflecting the amount of efflux during the experiment. Furthermore, the rate of dilution of the labeled molecule or complex by endogenous unlabeled molecule or complex can be calculated to determine the rate of the efflux component of reverse cholesterol transport in the subject. This process is exemplified in Example 1, infra.

A. Administering Isotopically Labeled Cholesterol or Cholesterol-Related Molecules or Cholesterol-Related Complexes.

Isotopically labeled cholesterol or cholesterol-related molecules or cholesterol-related complexes can be administered to a subject by various routes including, but not limited to, orally, parenterally, subcutaneously, intravascularly (e.g., intravenously or intra-arterially), intraperitoneally or intramuscularly.

The administered isotope-labeled cholesterol or cholesterol-related molecules or cholesterol-related complexes may be, but are not limited to, any of the following:
Isotope-labeled cholesterol
Isotope-labeled cholesterol suspended in a lipid emulsion
Isotope-labeled cholesterol associated with high-density lipoprotein (HDL) particles
Isotope-labeled cholesterol associated with low-density lipoprotein (LDL) particles
Isotope-labeled cholesterol associated with very-low-density lipoprotein (VLDL) particles
Isotope-labeled cholesterol associated with intermediate-density lipoprotein (IDL) particles
Isotope-labeled cholesterol associated with chylomicrons
Isotope-labeled cholesterol-ester
Isotope-labeled cholesterol-ester suspended in a lipid emulsion
Isotope-labeled cholesterol-ester associated with HDL particles
Isotope-labeled cholesterol-ester associated with LDL particles
Isotope-labeled cholesterol-ester associated with VLDL particles
Isotope-labeled cholesterol-ester associated with IDL particles
Isotope-labeled cholesterol-ester associated with chylomicrons

The isotope used to label the cholesterol or cholesterol-related molecule or cholesterol-related complex may be a stable isotope (e.g., ²H, ¹⁸O, or ¹³C), or it may be a radioisotope (e.g., ³H or ¹⁴C). The cholesterol or cholesterol-related molecule or cholesterol-related complex may have multiple different isotope labels. It may be labeled on multiple positions with the same isotope label. It may be labeled on multiple positions with multiple different labels. For example, one can label with ²H and ¹⁸O or ²H and ¹³C or any combination of labels.

One can also substitute a modified cholesterol or cholesterol-related molecule or complex for an isotope-labeled one. As long as the modified cholesterol or cholesterol-related molecule or cholesterol-related complex can be distinguished from its endogenous counterpart, the techniques can be practiced with a modified cholesterol or cholesterol-related molecule or cholesterol-related complex. An example of a modified cholesterol molecule is methyl-cholesterol, which is modified with a methyl group. In the case of methyl-cholesterol, a single methyl group is added to cholesterol in order to create a cholesterol molecule that is very similar to cholesterol, but can be distinguished by mass spectrometry, NMR or other methods such as laser spectrophotometry or laser spectroscopy (see infra). Measurement of the fraction of a molecule of interest that includes the additional methyl group can take the place of measurement of the isotopic content of the molecule of interest (see infra). Similarly, the molar percent excess would be expressed in terms of methylated molecule of interest (e.g., MPE of M_{methyl}). MPE calculations are discussed infra.

Methods for the preparation of the above listed isotope-labeled molecules or complexes, or other isotope labeled molecules or complexes are known to those of skill in the art.

The isotope-labeled cholesterol or cholesterol-related molecules or cholesterol-related complexes may be administered in a variety of modes. They can be administered continuously, repeatedly, discontinuously, or by other modes. In one embodiment, a known amount of isotope-labeled cholesterol in a lipid emulsion is infused at a constant rate for a length of time sufficient to achieve steady-state levels in plasma cholesterol.

B. Obtaining One or More Biological Samples.

After or during administration of the isotope-labeled cholesterol or cholesterol-related molecule or cholesterol-related complex, a biological sample is obtained. The frequency of biological sampling may vary depending on different factors. Such factors include, but are not limited to, the nature of the biological sample, ease and safety of sampling, biological rate constants and turnover kinetics of the cholesterol or cholesterol-related molecule or cholesterol-related complex, and the nature of a candidate therapy that is administered to a subject. In one embodiment, multiple biological samples are collected from a subject during and after the infusion of the isotope-labeled cholesterol or cholesterol-related molecule or cholesterol-related complex.

The nature of the biological sample may vary widely. In one embodiment, the sample is urine or feces. In another embodiment, the sample is blood. The sample is chosen in order to obtain a sufficient amount of cholesterol or cholesterol-related molecule or cholesterol-related complex (the molecule of interest), which is analyzed for its isotopic content or isotopic pattern. The molecule of interest varies with experimental design, and may be selected based on the choice of the isotope-labeled cholesterol or cholesterol-related molecule or cholesterol-related complex that is administered as described in section I-A, supra. A sample may contain multiple molecules of interest, and multiple samples may be taken in order to analyze a single molecule of interest. In one embodiment, the isotope-labeled cholesterol or cholesterol-related molecule or cholesterol-related complex is ¹³C-labeled cholesterol in a lipid emulsion, and the biological sample is a blood sample, which will contain cholesterol. The isotopic content or isotopic pattern of cholesterol in the blood will then be determined, as described, infra.

Subsequently, measurements of the isotopic content or isotopic pattern of the molecules of interest are made. This measurement may be made directly on the sample, or it may be made after processing the sample. In some cases, the sample may be processed extensively before the isotopic content or isotopic pattern is measured. The sample may be processed to isolate a particular cholesterol-related molecule or cholesterol-related complex, such as HDL or a subclass of HDL, and the isolated molecule or complex then may be further processed into a form suitable for mass spectrometric analysis.

Some of the techniques that may be applied to a biological sample to purify, partially purify, or isolate a cholesterol or cholesterol-related molecule or cholesterol-related complex include, but are not limited to, centrifugation, solvent-, salt-, or pH-based precipitation, high pressure liquid chromatography (HPLC), fast performance liquid chromatography (FPLC), reversed-phase chromatography, size exclusion chromatography, thin layer chromatography, gas chromatography, gel electrophoresis, ultrafiltration, ultracentrifugation, affinity chromatography, capillary electrophoresis, selective or differential proteolysis, differential chemical degradation, crystallization, recrystallization, limited proteolysis, limited chemical degradation, and/or any other methods of separating chemical and/or biochemical and/or macromolecular compounds, biomolecules or complexes known to those skilled in the art. Furthermore, methods of obtaining, purifying, and isolating cholesterol and/or cholesterol-related molecules and/or cholesterol-related complexes may be found, for example, in Cell Biology: A Laboratory Notebook (J. E. Cellis, ed., 1998); Current Protocols in Molecular Biology (F. M. Ausubel et al., eds, 1987); Short Protocols in Molecular Biology (Wiley and Sons, 1999), as well as other sources well known in the art.

Purified or partially purified cholesterol or cholesterol-related molecules or cholesterol-related complexes may be further processed for isotopic content or isotopic pattern analysis by techniques that include, but are not limited to, chemical hydrolysis, thermal hydrolysis, acid hydrolysis, chemical derivatization (e.g., acylation, acetylation), aqueous-organic extraction, chemical drying, vacuum drying, and others known in the art. Purified or partially purified cholesterol or cholesterol-related molecules or cholesterol-related complexes may be conjugated to other molecules prior to analysis. For instance, cholesterol may be derivatized to its trimethylsilyl derivative prior to isotopic content or isotopic pattern analysis.

In another embodiment, the isotopically labeled cholesterol or cholesterol-related molecule or cholesterol-related complex may be hydrolyzed or otherwise degraded to form smaller molecules. Hydrolysis methods may be any method known in the art, including, but not limited to, chemical hydrolysis (such as acid hydrolysis) and biochemical degradation. Hydrolysis or degradation may be conducted either before or after purification and/or isolation of the cholesterol or cholesterol-related molecule or cholesterol-related complex.

C. Measuring the Isotopic Content or Isotopic Pattern of Cholesterol or Cholesterol-Related Molecules or Cholesterol-Related Complexes.

The isotopic content or isotopic pattern of the cholesterol or cholesterol-related molecule or cholesterol-related complex of interest is then determined. The isotopic content or isotopic pattern may be determined by methods including, but not limited to, mass spectrometry, nuclear magnetic resonance (NMR) spectroscopy, laser spectrophotometry, laser spectroscopy, liquid scintillation counting, or other methods known in the art. The isotopic content or isotopic pattern may be measured directly, or may be analyzed after the cholesterol has been chemically or biochemically modified as described, supra.

Isotopic content or isotopic pattern in cholesterol or cholesterol-related molecules or cholesterol-related complexes may be determined by various mass spectrometric methods, including but not limited to, gas chromatography-mass spectrometry (GC-MS), isotope-ratio mass spectrometry, GC-isotope ratio-combustion-MS, GC-isotope ratio-pyrrolysis-MS, liquid chromatography-MS, electrospray ionization-MS, matrix-assisted laser desorption-time of flight-MS, Fourier-transform-ion-cyclotron-resonance-MS, cycloidal-MS, and the like.

Mass spectrometers convert molecules into rapidly moving gaseous ions which then are analyzed on the basis of their mass-to-charge ratios. The distributions of isotopes or isotopologues of ions, or ion fragments, may thus be used to measure the isotopic content or isotopic pattern in a plurality of molecules.

Generally, mass spectrometers include an ionization means and a mass analyzer. A number of different types of mass analyzers are known in the art. These include, but are not limited to, magnetic sector analyzers, electrospray ionization, quadrupoles, ion traps, time of flight mass analyzers, and Fourier transform analyzers.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, two or more mass spectrometers may be coupled (MS/MS) first to separate precursor ions, then to separate and measure gas phase fragment ions. These instruments generate an initial series of ionic fragments of a molecule, and then generate secondary fragments of the initial ions. The MS/MS fragmentation patterns and exact molecular mass determinations generated by mass spectrometry provide unique information regarding the chemical composition of molecules. An unknown molecule can be identified in minutes, by a single mass spectrometric analytic run. The library of chemical fragmentation patterns that is now available provides the opportunity to identify components of complex mixtures with near certainty. Such a technique may be used to analyze the isotopic content or isotopic pattern of a cholesterol or cholesterol-related molecule or cholesterol-related complex of interest without the need for any processing of the relevant biological sample (i.e., a direct measurement).

Different ionization methods are also known in the art. One key advance has been the development of techniques for ionization of large, non-volatile macromolecules. Techniques of this type have included electrospray ionization (ESI) and matrix assisted laser desorption/ionization (MALDI). These have allowed MS to be applied in combination with powerful sample separation introduction techniques, such as liquid chromatography and capillary zone electrophoresis.

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

In addition, where the isotope is radioactive, isotopic content or isotopic pattern or abundances may be measured using techniques known in the art for the measurement of radioisotopes, including, but not limited to, liquid scintillation counting, geiger counting, CCD based detection, film based detection, and others.

In general, the measurements contemplated herein can be carried out with a broad range of instrument types. The above list is non-limiting.

In the present disclosure, two classes of isotope-labeled molecules are contemplated. The first class is the isotope-labeled cholesterol or cholesterol-related molecule or cholesterol-related complex that is administered to the subject. The second class is the molecule of interest. The molecule of interest is the molecule whose isotopic content or isotopic pattern is subsequently measured. The molecule of interest is contained within a biological sample. Molecules of interest may be cholesterol or cholesterol-related molecules or cholesterol-related complexes. Molecules of interest may be labeled via the metabolic action of the subject. Molecules of interest may be the same as the isotope-labeled cholesterol or cholesterol-related molecule or cholesterol-related complex administered to the subject. In short, isotope-labeled cholesterol or cholesterol-related molecules or cholesterol-related complexes are administered to subjects, and the isotopic content or isotopic pattern of the molecule of interest is what is subsequently measured.

In general, the isotopic content or isotopic pattern of a cholesterol or cholesterol-related molecule or cholesterol-related complex in a biological sample derived from the present disclosure is expressed relative to the baseline isotopic content or isotopic pattern of the same molecule, prior to the administration of any isotope-labeled molecules. In order to determine a baseline isotopic content or isotopic pattern for the chosen cholesterol or cholesterol-related molecule or cholesterol-related complex (the molecule of interest), a sample can be taken before administration of the isotope-labeled cholesterol or cholesterol-related molecule or cholesterol-related complex, and the isotopic content or isotopic pattern of the molecule of interest can be analyzed in the baseline sample. Such a measurement is one means of establishing the naturally occurring isotopic content or isotopic pattern of the molecule of interest in the organism. In many cases, the baseline isotopic content or isotopic pattern can be estimated based on historical (existing) data from biological samples taken from subjects who received no labeled molecules of any type. This is especially true when an organism is part of a population of subjects having similar environmental histories. Additionally, such a baseline isotopic content or isotopic pattern may be estimated, using known average natural abundances of isotopes. For example, in nature, the natural abundance of ¹³C present in organic carbon is 1.11%. Methods of predicting such isotopomer frequency distributions are well known in the art and described in the literature (see infra).

The actual isotopic content or isotopic pattern may be calculated from data obtained as described, supra. These calculations can take many forms, depending on the amount of historical or baseline data available, the preference of the practitioner, the desired accuracy or precision of the measurements, the type of instrument used for the analysis, and other factors. Example calculations follow.

1. Measuring Relative and Absolute Mass Isotopomer Abundances.

Mass spectrometers measure the relative quantity of different mass molecules or atoms in a sample. These quantities are sometimes referred to as abundances. Measured mass spectral peak heights, or alternatively, the areas under the peaks, may be expressed as ratios toward the parent (zero mass isotope) isotopomer. It is appreciated that any calculation means which provide relative and absolute values for the abundances of isotopomers in a sample may be used in describing such data, for the purposes of the present disclosure. In one embodiment, the relative abundances of different mass isotopomers are measured by GC/MS and the molar percent excess of given isotopomer is calculated. In another embodiment, the relative abundances of different isotopes are measured at the atomic level by GC-combustion isotope ratio-mass spectrometry (GCC-IRMS), or GC-pyrrolysis-isotope ratio-mass spectrometry (GCP-IRMS), and the atom percent excess of a given isotopomer is calculated.

2. Calculating Isotopic Content or Isotopic Pattern.

a. Molar Percent Excess (MPE)

Isotopic content or isotopic pattern may be calculated from abundance data collected as described in section I-C-1, supra. In one embodiment, isotopic content or isotopic pattern is expressed as molar percent excess (MPE). To determine MPE, the practitioner first determines the fractional abundance of an isotopomer of the molecule of interest (the isotopomer is selected based on the nature of the molecule of interest and the nature of the isotope-labeled cholesterol or cholesterol-related molecule or cholesterol-related complex administered to the subject). This can be calculated from abundance data, such as that from GC/MS, using the following equation, which is a general form for the determination of fractional abundance of a mass isotopomer Mₓ:

$$Fractional abundance of {M}_{x} = \frac{Abundance {M}_{x}}{{\sum }_{i = 0}^{n} Abundance {M}_{i}} ,$$

where 0 to n is the range of nominal masses relative to the lowest mass (M₀) mass isotopomer in which abundances occur.

Once the fractional abundance is determined, it is compared to the baseline, historical baseline, theoretical baseline, or other such reference values (obtained as described, supra) in order to determine the MPE. This is calculated using the following equation:

$$MPE = {EM}_{X} = Δ fractional abundance = enrichment = {( {M}_{x} )}_{e} - {( {M}_{X} )}_{b} = {( \frac{{AbundanceM}_{X}}{{\sum }_{i = 0}^{n} {AbundanceM}_{i}} )}_{e} - {( \frac{{AbundanceM}_{X}}{{\sum }_{i = 0}^{n} {AbundanceM}_{i}} )}_{b} ,$$

where subscript e refers to enriched and b refers to baseline or natural abundance.

Once the MPE is determined, the fraction of newly synthesized molecules or the extent of dilution by endogenous molecules can be determined. In both cases, the MPE is compared to a value representing the maximum possible molar percent excess. In the case where a molecule of interest is produced by the metabolism of isotope-labeled precursor (e.g., the production of ²H₄-cholic acid from ²H₄-cholesterol), the MPE of the precursor may be measured and used directly or as a basis for calculation of a maximum potential MPE. The maximum potential MPE may also be determined from historical data, from calculations based on the amount of isotope label administered, from similar calculations that take into account properties of the subject (e.g., weight, body composition), from purely theoretical calculations, and from other combinations of estimation, measurement, and retrospective data analysis. The maximum possible MPE may also be determined by measuring the MPE in a separate biological sample that is known to contain fully labeled molecule of interest. In the case of dilution of label, the maximum possible MPE is based on the MPE of the administered isotope-labeled cholesterol or cholesterol-related molecule or cholesterol-related complex.

The applicant has considerable experience in the field of isotope label incorporation and isotopomer distribution, and has developed a number of technologies and modes of calculation relevant to the calculation and analysis of isotopic content or isotopic pattern. These include the Mass Isotopomer Distribution Analysis (MIDA), and are described extensively, particularly in U.S. Pat. Nos. 5,338,686, 5,910,403, and 6,010,846, which are hereby incorporated by reference in their entirety. Variations of MIDA and other relevant techniques are further described in a number of different sources known to one skilled in the art, including Hellerstein and Neese (1999), as well as Chinkes, et al. (1996), and Kelleher and Masterson (1992), and U.S. patent application Ser. No. 10/279,399, all of which are hereby incorporated by reference in their entirety.

In addition to the above-cited references, calculation software implementing the method is publicly available from Professor Marc Hellerstein, University of California, Berkeley.

b. Atom Percent Excess (APE.

Isotopic content or isotopic pattern may be calculated from abundance data collected as described, supra. In one embodiment, isotopic content or isotopic pattern is expressed as atom percent excess (APE). To determine APE, the practitioner first determines the fractional abundance of the isotope of interest in the molecule of interest (the isotope of interest is selected based on the nature of the molecule of interest and the nature of the isotope-labeled cholesterol or cholesterol-related molecule or cholesterol-related complex administered to the subject). This can be calculated from abundance data, such as that from GCC-IR-MS or GCP-IR-MS using the following equation, which is a general form for the determination of fractional abundance of an isotope I_{X}:

$$Fractional abundance of {I}_{x} = {A}_{x} = \frac{{AbundanceI}_{X}}{{\sum }_{i = 0}^{n} {AbundanceI}_{i}}$$

where 0 to n is the range of possible isotopes of the chosen atom in which abundances are measured.

Once the fractional abundance is determined, it is compared to the baseline, historical baseline, theoretical baseline, or other such reference values (obtained as described, supra) in order to determine the atom percent excess (APE). This is calculated using the following equation:

$$APE = Δ fractional abundance = enrichment = {( {A}_{X} )}_{e} - {( {A}_{X} )}_{b} = {( \frac{{AbundanceI}_{X}}{{\sum }_{i = 0}^{n} {AbundanceI}_{i}} )}_{e} - {( \frac{{AbundanceI}_{X}}{{\sum }_{i = 0}^{n} {AbundanceI}_{i}} )}_{b}$$

where subscript e refers to enriched and b refers to baseline or natural abundance.

Once the APE is determined, the fraction of newly synthesized molecules or the extent of dilution by endogenous molecules can be determined. This is carried out as described, supra, but may require additional calculations in the case of the theoretical maximum APE. Such calculations are known to those of skill in the art.

3. Types of Isotopic Content or Isotopic Pattern.

In the present disclosure, isotopic content or isotopic pattern is often expressed as MPE or as APE. Molar percent excess is sometimes written as EM_{X}, and refers to the molar percent excess of a given mass (with respect to all possible masses of the molecule being analyzed as compared to the baseline sample, historical baseline data, or predicted baseline values). Many combinations of administered isotope-labeled cholesterol or cholesterol related molecules or cholesterol-related complexes and molecules of interest are contemplated in the present disclosure. Non-limiting scenarios intended to illustrate the possible range of methodologies follow:

Scenario 1: ¹³C₂-cholesterol (defined as cholesterol containing two atoms of ¹³C in place of the predominantly ¹²C atoms in natural abundance molecules) may be administered to a subject, and blood cholesterol may be subsequently analyzed by GC/MS to determine the isotopic content or isotopic pattern of cholesterol. In such a case, when combined with baseline or similar data, the EM₂ (representing, in addition to other naturally occurring isotopologues, cholesterol labeled with two atoms of ¹³C) may be determined and represents a relevant measurement of isotopic content or isotopic pattern. Alternatively, cholesterol from the same sample may be analyzed by gas-chromatography/pyrolysis/isotope ratio mass spectrometry in order to determine the APE of ¹³C, which would also be a relevant measurement of isotopic content or isotopic pattern.

Scenario 2: ²H₄-cholesterol may be administered to a subject, and blood cholesterol may be subsequently analyzed by GC/MS to determine the isotopic content or isotopic pattern of cholesterol. In such a case, when combined with baseline or similar data, the EM₄ (representing, in addition to other naturally occurring isotopologues, cholesterol labeled with four atoms of ²H) may be determined and represents a relevant measurement of isotopic content or isotopic pattern. Alternatively, the cholesterol from the same sample may be analyzed by gas-chromatography/pyrolysis/isotope ratio mass spectrometry in order to determine the APE of ²H, which would also be a relevant measurement of isotopic content or isotopic pattern.

Scenario 3: ²H₄-cholesterol may be administered to a subject, and blood cholesterol-ester may be subsequently analyzed by GC/MS to determine the isotopic content or isotopic pattern of cholesterol. In such a case, when combined with baseline or similar data, the EM₄ (representing, in addition to other naturally occurring isotopologues, cholesterol-ester labeled with four ²H atoms) may be determined and represents a relevant measurement of isotopic content or isotopic pattern. Alternatively, cholesterol ester from the same sample may be analyzed by gas-chromatography/pyrolysis/isotope-ratio mass spectrometry in order to determine the APE of ²H, which would also be a relevant measurement of isotopic content or isotopic pattern.

D. Calculating the Rate of Dilution of Isotopically Labeled Cholesterol or Cholesterol-Related Molecules or Cholesterol-Related Complexes.

The isotopic content or isotopic pattern of the cholesterol or cholesterol-related molecule or cholesterol-related complex of interest measured in the biological sample may be compared to the isotopic content or isotopic pattern of the isotopically labeled cholesterol or cholesterol-related molecule or cholesterol-related complex that was administered to the subject. This comparison allows for the calculation of dilution by unlabeled endogenous molecule of interest. Dilution equations are known in the art and are described, for example, by Hellerstein et al. (1992), supra. The rate of dilution or Ra is then used to determine the molecular flux rate of tissue cholesterol into blood lipoproteins, which corresponds to the efflux component of RCT.

$$DilutionRate = \frac{InfusionRate ( labeledCholesterol )}{Enrichment ( LabeledCholesterol )} - InfusionRate ( LabeledCholesterol )$$

In one embodiment, stable-isotope labeled cholesterol suspended in a lipid emulsion is infused for a period of time sufficient to achieve steady state levels of plasma cholesterol enrichment. Plasma samples are taken periodically during the infusion, and the isotopic content or isotopic pattern of cholesterol in the plasma samples is measured as described, supra. The change in isotopic content or isotopic pattern over time is used to determine the steady state level of isotopic content or isotopic pattern as well as the half life of plasma cholesterol. In this case, the rate of dilution of plasma cholesterol may be determined directly using the equation above. The dilution rate is the same as the rate of cholesterol efflux, and represents the rate of the efflux (Ra) component of RCT in the subject under study.

In other embodiments of the disclosure, in particular those where steady state enrichment of the cholesterol or cholesterol-related molecule or cholesterol-related complex of interest is not achieved, the calculation of the efflux component of RCT is more complex. However, as long as the appearance of labeled cholesterol or cholesterol-related molecules or cholesterol-related complexes in plasma can be described mathematically (amount in plasma from infusion as a function of time), the efflux component of RCT can be measured using the methods of the present disclosure, by use of equations and mathematical techniques that are well known in the art.

E. Calculating of Pool Size of the Rapidly Turning-Over Cholesterol Pool in the Body.

The rapid turnover pool size can be calculated by the methods disclosed herein, by use of an equation derived from the efflux model:
Pool size (mg/kg)=Ra (mg/kg/hr)/k (hr⁻¹)
II. Determining the Molecular Flux Rate of the Hepatic or Excretory Component of RCT in a Subject.

In another aspect, the present disclosure is directed to measuring the hepatic or excretory component of RCT. In the hepatic or excretory component of RCT, cholesterol or cholesterol-related molecules or cholesterol-related complexes are converted into bile acids then secreted into the gut lumen, or secreted directly as neutral sterols (biliary neutral sterols include bile cholesterol). The contribution of RCT to bile acids or neutral sterols, or both, in excreta may be determined.

A. Administering One or More Isotopically Labeled Cholesterol or Cholesterol-Related Molecules or Cholesterol-Related Complexes.

A stable-isotope labeled cholesterol or cholesterol-related molecule or cholesterol-related complex is administered as described, supra. Any mode, route of administration, or quantity of stable-isotope label can be used as described, supra.

B. Obtaining One or More Biological Samples.

After or during administration of the isotope-labeled cholesterol or cholesterol-related molecule or cholesterol-related complex, a biological sample is obtained. When measuring the hepatic or excretory component of RCT, the sample will preferably contain fecal bile acids and fecal neutral sterols. The number of biological samples, their frequency, and their timing can vary depending on different factors. Such factors include, but are not limited to, the nature of the biological sample, ease and safety of sampling, biological rate constants or metabolic behavior of the cholesterol or cholesterol-related molecule or cholesterol-related complex of interest, and the nature of a candidate therapy that is administered to a subject. There may be only one sample. It may be collected during the administration of the isotope-labeled cholesterol or cholesterol-related molecule or cholesterol-related complex, or it may be collected after administration. If collected after, the sample may be collected immediately after administration, or a period of time may pass after administration prior to sample collection. In one embodiment, multiple biological samples may be collected from a subject, including stool, urine, and blood. In another embodiment, a single biological sample of blood may be obtained. The biological samples may be processed extensively as described, supra. In one embodiment, the bile acids and bile neutral sterols may be isolated from the biological sample or samples and derivatized for mass spectrometric analysis.

C. Measuring the Isotopic Content or Isotopic Pattern of Bile Acids and Neutral Sterols.

Isotopic content or isotopic pattern of the bile acids and neutral sterols may be determined and compared to the isotopic content or isotopic pattern of the bile acids or neutral sterols prior to administration in order to determine the APE or MPE of the bile acids and neutral sterols. This APE or MPE is then compared to the maximum possible APE or MPE for the bile acids or neutral sterols (this maximal value may be determined by measuring in the subject after an appropriate period of time, the isotopic content or isotopic pattern of the isotope-labeled cholesterol or cholesterol-related molecule or cholesterol-related complex that is being converted to bile acids or neutral sterols. It may also be calculated from historical data, or derived from the literature. Division of the observed APE or MPE for the bile acid or neutral sterol of interest by the maximum possible APE or MPE of the same bile acid or bile neutral sterol yields the fraction of the bile acid or bile neutral sterol that is derived from RCT. The transport or conversion of the isotope-labeled cholesterol or cholesterol-related molecule to neutral sterols is also calculated. In this manner, the hepatic or excretory component of RCT (FIG. 1) can be measured.

Additionally, when the biological sample is stool, the isotopic content or isotopic pattern of cholesterol itself may be determined.

Fecal cholesterol represents an important fraction of the cholesterol removed from the body by RCT. As stated, supra, the isotopic content or isotopic pattern of fecal cholesterol can be measured when the biological sample is stool. Once secreted, a portion of intestinal lumen cholesterol is reabsorbed from the gastrointestinal tract, and re-enters the plasma pool of circulating cholesterol. The mixing of intestinal cholesterol with circulating cholesterol in the plasma means that the isotopic content or isotopic pattern of excreted cholesterol cannot reliably be directly measured in any biological sample other than stool. In order to overcome this limitation, the isotopic content or isotopic pattern of gut-specific metabolites of cholesterol can instead be determined from non-stool biological samples. Bile cholesterol is converted by gastrointestinal microbes into a number of metabolites including coprostanol, coprostanone, cholestanol, cholestanone and epicoprostanol. These cholesterol metabolites are also reabsorbed and are distributed throughout the body, making their way into other tissues and fluids, including, but not limited to, the blood and urine. In one embodiment, the isotopic content or isotopic pattern of these cholesterol metabolites is measured in urine. In another embodiment, the isotopic content or isotopic pattern of these cholesterol metabolites is measured in blood. The isotopic content or isotopic pattern of such cholesterol metabolites is the same as that found in bile cholesterol, and such a measurement can take the place of the direct measurement of the isotopic content or isotopic pattern of bile cholesterol in stool. In another embodiment, the isotopic content or isotopic pattern of gut-specific cholesterol metabolites and bile acids are both determined from a single sample of urine.

Isotopic content or isotopic pattern, fractional synthesis or conversion rates, and MPE or APE values are determined as described, supra.

D. Measurement of Bile Acid Pool Size.

The total amount of bile acids (bile acid pool size) may be measured concurrently, before or after measurement of the hepatic or excretory component of RCT. This serves the purpose of allowing the practitioner to compare results between different subjects with different bile acid pool sizes, and allows for the determination of the absolute mass of isotope-labeled cholesterol or cholesterol-related molecule of interest that is converted into bile acids. This absolute mass is the product of the fraction of bile acids derived from the administered isotope-labeled cholesterol or cholesterol-related molecule (determined as described, supra) multiplied by the bile acid pool size.

The bile acid pool size is determined, for example, by the dilution method, wherein a known amount of an isotope-labeled bile acid is administered to a subject, and after a period of time, the isotopic content or isotopic pattern of bile acid in the subject is determined from a sample or multiple samples. The amount of dilution of the isotope-labeled bile acid (i.e., the reduction in APE or MPE for the bile acid in question) or back-extrapolation from the curve showing the rate of reduction of APE or MPE is used to calculate the total mass of bile acid in the subject.

1. Administration of Isotope-Labeled Bile Acids.

One or more isotope-labeled bile acids may be administered to a subject. If measurement of the bile acid pool size is to be carried out concurrently with the determination of the hepatic or excretory component of RCT, the isotopically-labeled bile acids will be labeled differently than the isotopically labeled cholesterol or cholesterol-related molecule or cholesterol-related complex, e.g., the bile acid will be labeled with four deuterium (²H) atoms, and the cholesterol will be labeled with two ¹³C atoms. Any number of isotopes may be used as long as the bile acid and cholesterol or cholesterol-related molecule or cholesterol-related complex is labeled with different isotopes. Alternatively, the bile acids can be labeled with the same isotope as isotope-labeled cholesterol or cholesterol-related molecule or cholesterol-related complex, but administered in a manner that is distinguishable from the manner used to administer the cholesterol or cholesterol precursor (e.g., different times, pulse, stopping vs. continuous and other distinguishable features well known to those skilled in the art).

Suitable isotope-labeled bile acids include cholic acid, chenodeoxycholic acid, deoxycholic acid, and lithocholic acid. In one embodiment the labeled bile acids may be cholic acid or chenodeoxycholic acid. Isotopes that may be used for labeling the bile acids include, but are not limited to, ²H, ¹³C, or ¹⁸O.

The isotope-labeled bile acids may be administered simultaneously with, or separately from, the isotope-labeled cholesterol or cholesterol-related molecules or cholesterol-related complexes. The isotope-labeled bile acids are administered in a suitable carrier at a predetermined volume and isotopic content or isotopic pattern. Suitable carriers include saline solution, triglyceride emulsions and intralipids. The administration of labeled bile acids to subjects may be by any number of routes, as described, supra. In one embodiment, bile acids are administered orally.

2. Obtaining a Biological Sample.

After a period of time, a biological sample containing bile acids is obtained. The period of time between administration of isotope-labeled bile acids and collection of a biological sample may vary, depending on the route and mode of administration, the nature of the subject (e.g., the species or disease state of the subject), the choice of isotope-labeled bile acid, and other factors known to those of skill in the art. The biological sample may be blood, stool, urine, or any other type of biological sample. The biological sample may be the same sample as collected for other measurements of isotopic content or isotopic pattern, such as for the determination of the hepatic or excretory component of RCT (see section II-B, supra)

Once collected, the sample may be processed or partially processed, and bile acids may be purified, isolated, or partially purified, as described, supra. Generally, such manipulations are known in the art.

3. Determining the Isotopic Content or Isotopic Pattern of Bile Acids.

The isotopic content or isotopic pattern of bile acids in the biological sample is measured and calculated as described, supra. The bile acid of interest is determined based on the nature of the isotope-labeled bile acid administered to a subject. For example, if a subject receives a known volume and concentration of ²H₄-cholic acid, then a suitable bile acid of interest would be cholic acid, and the MPE of the M₄ isotopomer would be determined. Alternatively, the APE of ²H may be determined.

4. Calculating the Bile Acid Pool Size.

The pool size of the bile acids in the subject may be determined using the following equation:

$$BileAcidPoolSize ( g ) = Mass {( g )}_{adm} \frac{{MPE}_{adm}}{{MPE}_{sample}} ,$$

where Mass(g)_{adm} is the mass in grams of isotope-labeled bile acid administered to the subject, MPE_{adm} is the molar percent excess of the stable-isotope-labeled bile acid administered, and MPEₛₐₘₚₗₑ is the peak or maximal molar percent excess of the same bile acid isotopomer in the biological sample.

An alternate form of this equation is:

$$BileAcidPoolSize ( g ) = Mass {( g )}_{adm} \frac{{APE}_{adm}}{{APE}_{sample}} ,$$

where Mass(g)_{adm} is the mass in grams of isotope-labeled bile acid administered to the subject, APE_{adm} is the atom percent excess of the isotope used to label the isotope-labeled bile acid in the administered isotope-labeled bile acid, and APEₛₐₘₚₗₑ is the peak or maximal atom percent excess of the same isotope in the same bile acid in the biological sample.

Alternatively, the peak isotopic enrichment of the isotope-labeled bile acid in the bloodstream can be extrapolated from the shape of the label die-away curve, by methods well known in the art. The pool size of the bile acid in the body can then be calculated by the dilution method.

E. Measurement of the Contribution of de Novo Cholesterol Synthesis to Bile Acids, Neutral Sterols and Cholesterol.

Optionally, the contribution of de novo synthesized cholesterol to bile acids, neutral sterols and cholesterol may be determined simultaneously while measuring the hepatic or excretory component of RCT. In a portion of the hepatic or excretory component of RCT, cholesterol and/or cholesterol-related molecules and/or cholesterol-related complexes are converted to bile acids, or secreted as neutral sterols, a process whose measurement is described in sections II-A through II-C, supra. Some bile acids and neutral sterols, however, are derived from cholesterol that is synthesized de novo in the liver or other tissues during the period of measurement of RCT, rather than derived from pre-existing cholesterol removed from other tissues. De novo cholesterol synthesis is a process which can also be measured by isotope-based techniques as described herein. Measurement of de novo cholesterol synthesis and its contribution to bile acids may be relevant to understanding the manner in which a candidate therapy influences disease and may provide complementary or supportive information concerning RCT fluxes in the individual. Concurrent measurement of the hepatic or excretory component of RCT and the contribution of de novo cholesterol synthesis to bile acids or neutral sterols in a subject improves the measurement of the hepatic or excretory component of RCT. Bile acids and neutral sterols derived from the hepatic or excretory component of RCT are measured as described in sections II-A through II-C, supra. Bile acids derived from de novo cholesterol synthesis are measured as described, infra. Remaining bile acids are those already present or derived from bile acids already present at the time of commencement of the experiment.

Measurement of de novo cholesterol synthesis and its contribution to bile acids, neutral sterols or cholesterol is measured by administering one or more isotope-labeled cholesterol precursors. After a period of time, during or after the administration of the one or more isotope-labeled cholesterol precursors, one or more biological samples comprising bile acids, neutral sterols or cholesterol are collected. The isotopic content or isotopic pattern of the bile acids, cholesterol, neutral sterols, or cholesterol metabolites in these samples is determined as described, supra, and the resulting data used to calculate the fraction or mass of bile acids derived from newly synthesized cholesterol.

1. Administering an Isotope-Labeled Cholesterol Precursor.

Modes of administering the one or more isotope-labeled cholesterol precursors may vary, depending upon the absorptive properties of the isotope-labeled cholesterol precursor and the specific biosynthetic pool into which it is targeted. Precursors may be administered via any of the routes previously contemplated, supra, or by other routes known in the art.

In one embodiment, the mode of administration is one that produces a steady state level of precursor within the biosynthetic pool and/or in a reservoir supplying such a pool for at least a transient period of time. Intravascular or oral routes of administration are commonly used to administer such precursors to subjects, including humans. Other routes of administration, such as subcutaneous or intramuscular administration, optionally when used in conjunction with slow release substrate compositions, are also appropriate. Compositions for injection are generally prepared in sterile pharmaceutical excipients, which are well known to those of skill in the art.

As is discussed herein, administration can be done continuously (e.g., up to and/or including the time of sampling) or discontinuously (either as a single dose over time or multiple doses). When discontinuous administration is done, the time of the individual administrations can either be the same or different.

Examples of isotope-labeled precursors are discussed in detail in U.S. patent application Ser. No. 11/064,197, incorporated herein by reference in its entirety, and in particular in section IV-B-1-a-2, titled “precursor molecules (isotope-labeled substrates)” and in U.S. Pat. No. 5,338,686, titled “Method for measuring in vivo synthesis of biopolymers”, incorporated herein by reference in its entirety. Isotope-labeled cholesterol precursors may be stable-isotope labeled molecules that, when administered to a subject result in the incorporation of isotope into de novo synthesized cholesterol. The isotope-labeled cholesterol precursor is chosen such that cholesterol, neutral sterols or bile acid molecules that are derived from the isotope-labeled precursor are isotopically distinct from those derived from the isotope-labeled cholesterol or cholesterol-related molecule or cholesterol-related complex administered for the determination of the hepatic or excretory component of RCT (sections II-A through II-C, supra), and are also isotopically distinct from the isotope-labeled bile acid administered in order to measure bile acid pool size (section II-D, supra). Alternatively, the timing and route of administration of the isotope-labeled cholesterol precursor may be varied in order to allow for the same distinction to be made. In one embodiment, the isotope-labeled cholesterol precursor is deuterium labeled water (²H₂O). In another embodiment, the isotope-labeled cholesterol precursor is H₂¹⁸O.

2. Obtaining One or More Biological Samples.

After a period of time, one or more a biological samples containing bile acids, bile neutral sterols, or cholesterol may be obtained. The period of time between administration of the isotope-labeled cholesterol precursor and collection of a biological sample may vary, depending on the route and mode of administration, the nature of the subject, the choice of isotope-labeled cholesterol precursor, and other factors known to those of skill in the art. The one or more biological samples may be blood, stool, urine, or any other type of sample. The one or more biological samples may be the same sample as collected for other measurements of isotopic content or isotopic pattern, such as for the determination of the hepatic or excretory component of RCT (see section II-B, supra), or for the determination of bile acid pool size (see section II-D, supra).

Once collected, the sample may be processed or partially processed, and bile acids may be purified, isolated, or partially purified, as described, supra. Generally, such manipulations are known in the art.

3. Measuring the Isotopic Content or Isotopic Pattern of Bile Acids or Neutral Sterols.

The isotopic content or isotopic pattern of the molecule of interest (bile acids or cholesterol or other cholesterol-related molecules or cholesterol-related complexes) is measured and calculated as described, supra. The contribution of de novo cholesterol synthesis to bile acids is measured by determining the isotopic content or isotopic pattern of bile acids. The contribution of de novo cholesterol synthesis to neutral sterols is measured by determining the isotopic content or isotopic pattern of the neutral sterols. In one embodiment, both are determined.

In one aspect of the disclosure, the isotopic content or isotopic pattern of bile acids and metabolites of cholesterol derived from the action of intestinal microbes on bile cholesterol are measured in urine or blood. As described, supra, cholesterol in non-stool biological samples cannot be analyzed directly in order to measure the isotopic content of fecal cholesterol. However, free cholesterol is converted by gastrointestinal microbes into a number of metabolites including coprostanol, coprostanone, cholestanol, cholestanone and epicoprostanol. These are also reabsorbed and appear in other tissues and bodily fluids, including the blood or urine. These metabolites are only formed from intestinal cholesterol, and so have an isotopic content or isotopic pattern that reflects intestinal cholesterol. As such, measuring the isotopic content or isotopic pattern of these cholesterol metabolites in urine represents a method for measuring the isotopic content or isotopic pattern of bile cholesterol or other neutral sterols.

4. Calculating the Contribution of de Novo Cholesterol Synthesis to Bile Acids and Neutral Sterols.

The isotopic content or isotopic pattern of bile acids or neutral sterols or bile cholesterol metabolites may be compared to the measured or known concentration of the isotope-labeled cholesterol precursor in order to determine the fraction of neutral sterols that are newly synthesized, or the fraction of bile acids that are derived from newly synthesized cholesterol. The calculation of the fraction is carried out as described supra, or as described previously in U.S. patent application Ser. No. 11/064,197, which is herein incorporated by reference in its entirety, and in U.S. Pat. No. 5,338,686, titled “Method for measuring in vivo synthesis of biopolymers” which is herein incorporated by reference in its entirety. Such calculations are also described extensively in a number of publications known to those of skill in the art.

F. Calculating the Activity of the Hepatic or Excretory Component of RCT

The activity of the hepatic or excretory component of RCT (i.e., the rate of conversion or secretion of administered cholesterol, to include cholesterol-related molecules or cholesterol-related complexes, or blood cholesterol to an excreted sterol product of interest; the mass of cholesterol, to include cholesterol-related molecules or cholesterol-related complexes, or blood cholesterol secreted; or the fraction or bile acids and/or neutral sterols derived from RCT) may be calculated using the isotopic content or isotopic pattern of the isolated bile acids or neutral sterols. The isotopic content or isotopic pattern of the bile acids may be compared to the total amount of isotopically-labeled cholesterol or cholesterol-related molecule or cholesterol-related complex administered to the subject (i.e., the isotopic recovery of administered label in the products) of interest). If bile acid pool size data is available, the recovery of administered labeled cholesterol or cholesterol-related molecule or cholesterol-related complex in bile acids is multiplied by the mass of the bile acid pool, giving a quantity of mass transferred by the hepatic or excretory component of RCT. If available, the isotopic content or isotopic pattern of bile acids as derived from a differently-labeled cholesterol precursor can be used to calculate, in a similar manner, the contribution of de novo synthesized cholesterol (DNC) to bile acid synthesis. The contributions to fecal neutral sterols from RCT and DNC are similarly calculated.

III. Determining the Plasma Component of RCT

The plasma component of RCT involves a variety of metabolic and transport steps (FIG. 3). Measurement of the plasma component of RCT is carried out by administering an isotope-labeled cholesterol or cholesterol-related molecule or cholesterol-related complex, and then subsequently measuring the isotopic content or isotopic pattern of a different cholesterol or cholesterol-related molecule or cholesterol-related complex. This “different” cholesterol or cholesterol-related molecule or cholesterol-related complex is an in vivo conversion product of the administered molecule; i.e., it is produced in vivo. Thus, the activity or rate of a given component of RCT can be measured. For example, the conversion of cholesterol to cholesterol ester (LCAT activity, FIG. 3) can be determined by administering isotope-labeled cholesterol and measuring the isotopic content or isotopic pattern of cholesterol ester. Similarly, the transfer of cholesterol ester from HDL to LDL or VLDL (CETP activity, FIG. 3) can be measured by administering isotope-labeled cholesterol ester in complex with HDL, and measuring the isotopic content or isotopic pattern of cholesterol ester in LDL or VLDL. The sum of the multiple steps of plasma RCT can be measured, for instance, by administering isotope-labeled cholesterol in complex with HDL, and then measuring the isotopic content or isotopic pattern of cholesterol ester from LDL or VLDL (i.e., simultaneous determination of the sum of LCAT and CETP activity).

In the plasma component of RCT, various types of cholesterol or cholesterol-related molecules or cholesterol-related complexes are converted into different types of cholesterol or cholesterol-related molecules or cholesterol-related complexes. To measure the plasma component of RCT, one type is administered, and another (the molecule of interest) is measured.

1. Administering Isotopically Labeled Cholesterol or Cholesterol-Related Molecules or Cholesterol-Related Complexes.

An isotope-labeled cholesterol or cholesterol-related molecule or cholesterol-related complex is administered as described, supra. Any mode, route of administration, or quantity of isotope label may be used, as described, supra. In one embodiment, the route of administration is intravenous. The choice of labeled molecule or complex is made based on what part of the plasma component of RCT one desires to measure.

2. Obtaining One or More Biological Samples.

After a period of time, a biological sample is obtained. The nature and timing of the biological sample obtained is determined by which part of the plasma component of RCT is being measured. In a preferred embodiment, the biological sample is a blood sample. More than one biological sample may be obtained.

3. Measuring Isotopic Content or Isotopic Pattern of Molecules of Interest.

Isotopic content or isotopic pattern is determined for the molecule of interest as described, supra. The molecule of interest is an in vivo conversion product of the administered isotopically-labeled molecule or complex.

4. Calculating the Rate of the Plasma Component of RCT

A standard precursor-product relationship may exist between the administered isotope-labeled cholesterol or cholesterol-related molecule or cholesterol-related complex and the downstream molecule of interest. Such a relationship is well known in the art. The precursor pool isotopic content or isotopic pattern is either estimated based on the amount of label administered, or is determined by use of historical data or other data, or it is measured directly. The fractional contribution of the isotope-labeled cholesterol or cholesterol-related molecule or cholesterol-related complex that was administered to the cholesterol or cholesterol-related molecule or cholesterol-related complex of interest is determined as described, supra.

IV. Summary:

A. Calculation of Cholesterol Efflux/Mobilization Rate from Tissues into Blood, by use of the Dilution Technique and the Plateau Principle.

Dilution of an infused tracer reveals rate of appearance (Ra, efflux, turnover) of the pool infused into (or in rapid communication with it) and is robustly performed when an isotopic plateau can be attained and demonstrated. There are many routine examples of this in metabolic research (e.g., Ra glucose, FA, glycerol, amino acids). This approach has not been used before for cholesterol, however, for several reasons. First, there had not been a physiologic rationale for measuring Ra cholesterol, before the discovery of a complex and regulated cholesterol efflux system (transporters, plasma acceptors, docking proteins, etc.). Second, cholesterol is poorly soluble in physiologic saline solution and is therefore not easy to administer by constant infusion. Moreover, the time required to achieve a plateau in plasma cholesterol specific activity or enrichment was also not clear, and might have taken days or weeks.

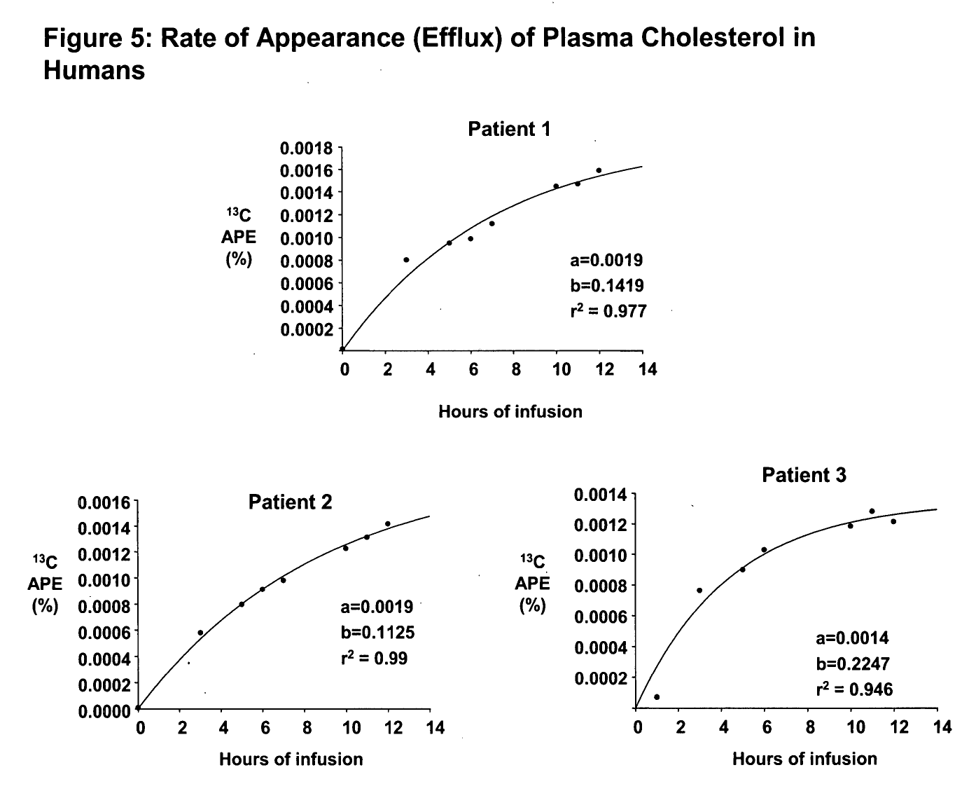

A key discovery was the observation of a plateau in plasma cholesterol enrichments, in humans and experimental animals. The achievement of plateau (see infra and FIG. 5) supports the existence of a functionally discrete and measurable rapidly turning-over pool, and allows calculation of Ra or efflux/mobilization rate of cholesterol from peripheral tissues into this rapidly turning-over pool, which is in communication with the blood (FIG. 4). This represents a major discovery, methodologically.

Another key discovery is that labeled cholesterol need not be administered in the form of HDL-complexes or other lipoprotein associated particles, but can be administered in the form of free cholesterol, to measure efflux/mobilization (Ra) and excretion efficiency (see infra and FIG. 5).

Efflux rate (Ra) represents the number of molecules entering the rapid turnover pool from the large, intracellular storage pool per hour—i.e., mobilized free cholesterol in and out of the storage pool (“flush-rate”). This represents an important and new metric of RCT in its own right (FIGS. 1 and 3-7) and also allows correction of label recovery in fecal sterols for influx/efflux across peripheral tissues (see infra).

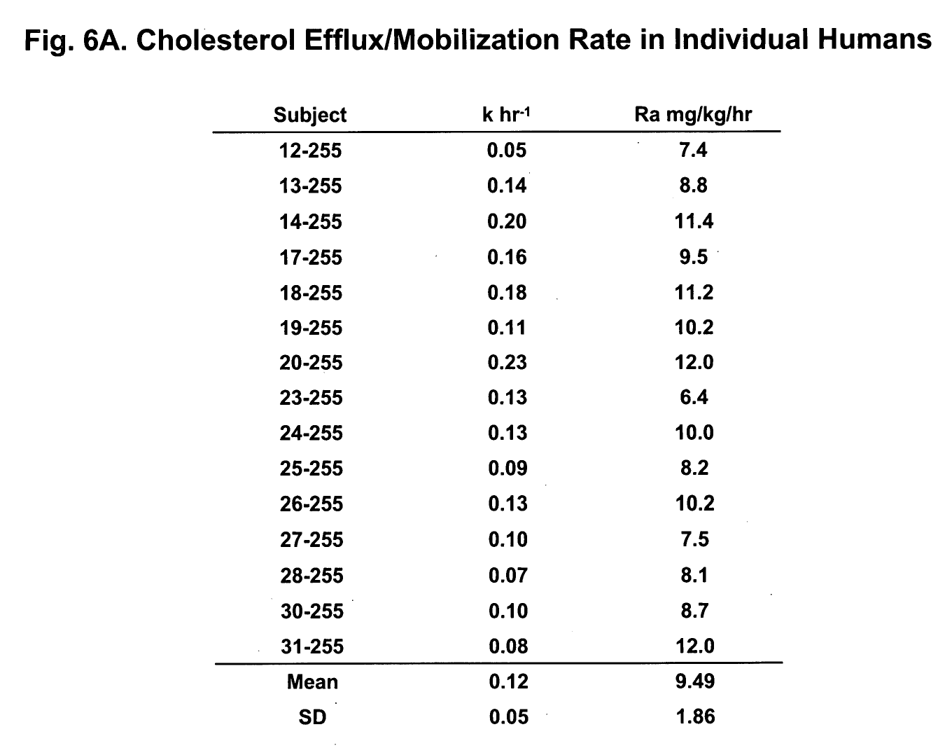

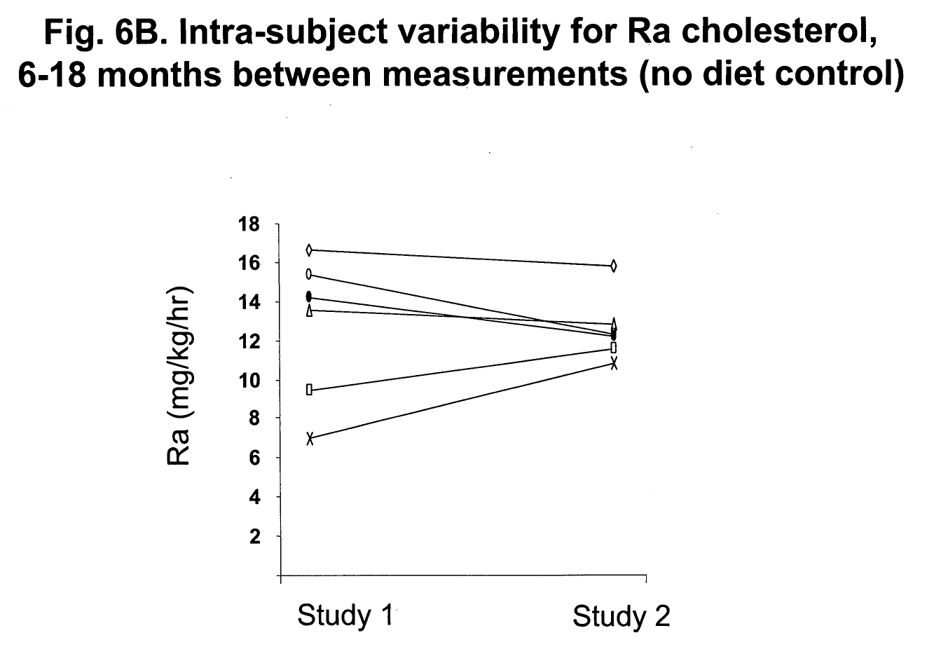

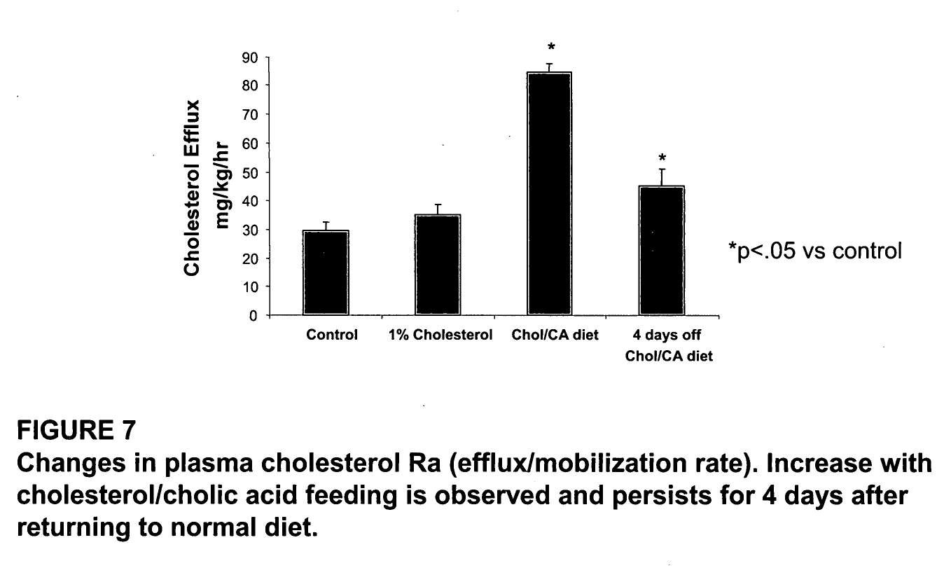

The data also demonstrate modulation by dietary loading of tissues with cholesterol (FIG. 7). Both Ra and pool size show reasonable but not excessive inter-individual variability in humans (e.g., standard deviation of about 20% [FIG. 6A], consistent with many regulated physiologic parameters), and suggesting that it is a modifiable parameter. Also, the pool size (which can be calculated as Ra/k) is approximately 5-10 g, consistent with Schwartz's data (6 g) (Schwartz et al, J Clin Invest)).

B. Correction of Labeled Cholesterol Excretion Efficiency into Fecal Sterols for Influx/Efflux Across Peripheral Tissues.

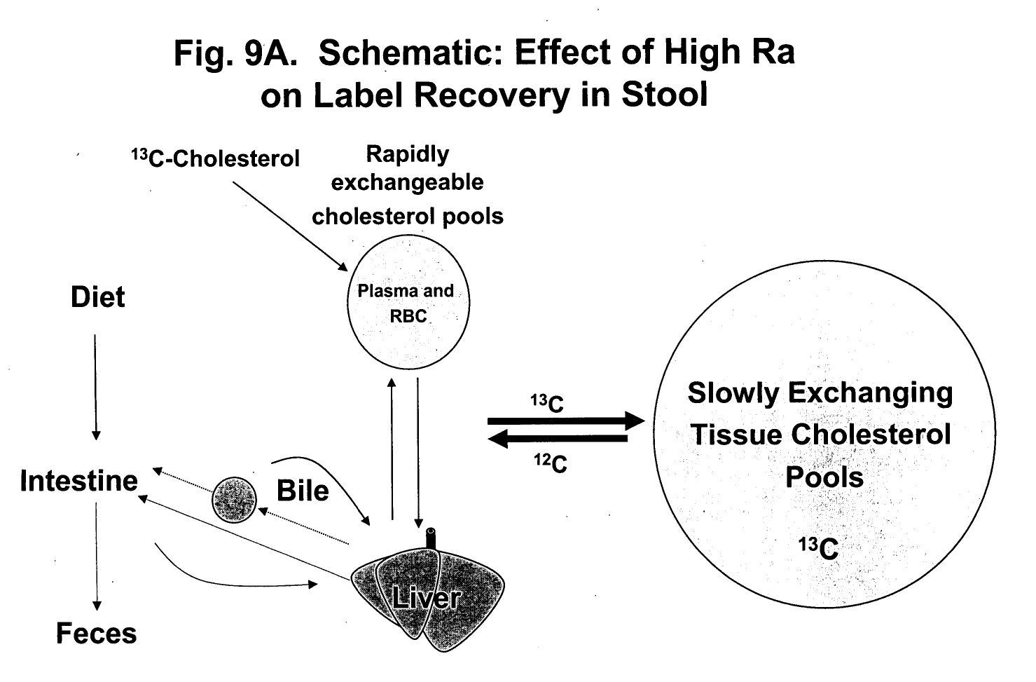

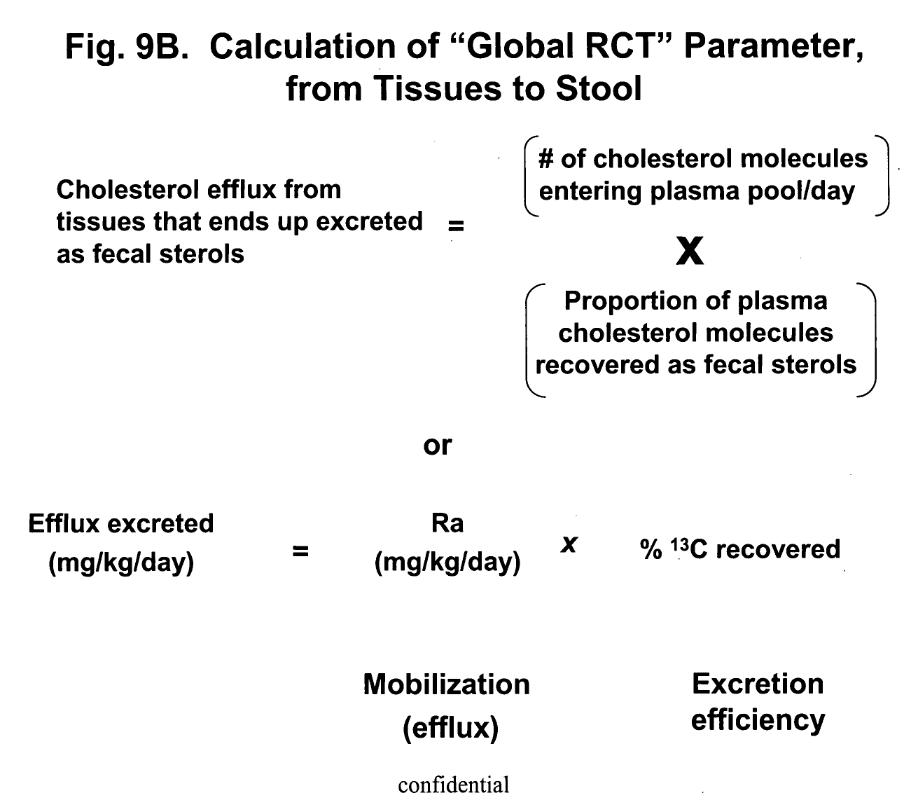

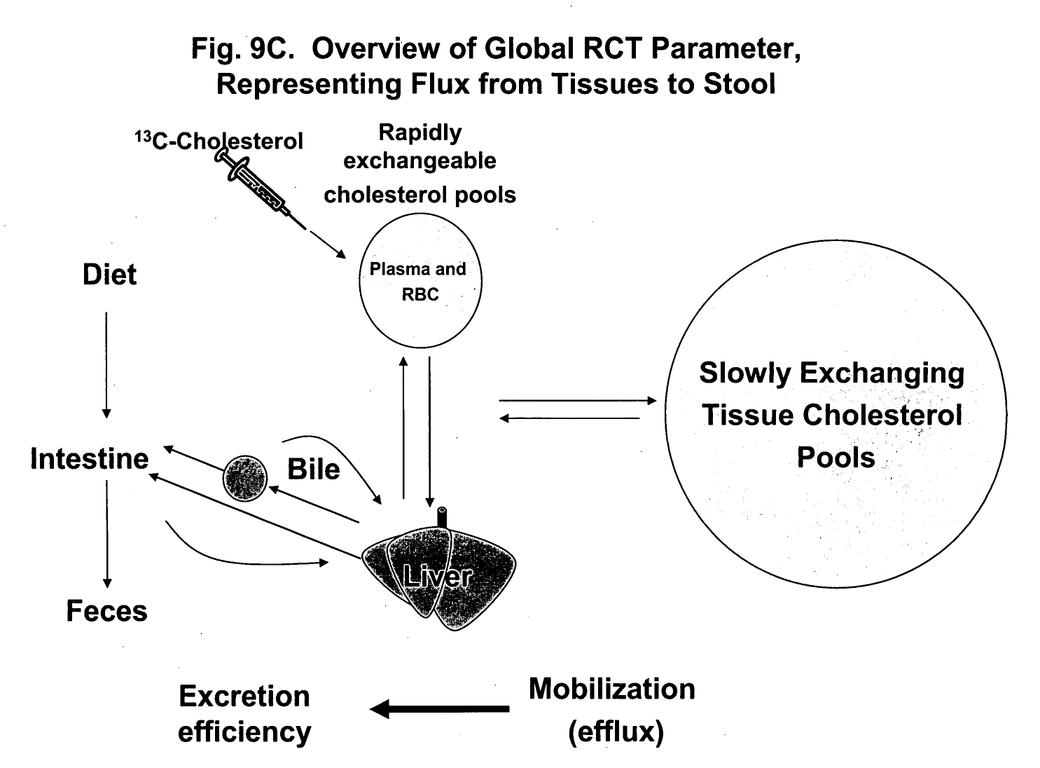

Label recovery of administered plasma cholesterol in stool represents the fractional excretion efficiency and rate of flux from plasma cholesterol to fecal sterols (arm 2 of RCT, see FIG. 1). This measurement is potentially confounded by changes in plasma cholesterol turnover (efflux/influx from and into tissues), however. If administered label exchanges rapidly in and out of the slow turnover tissue pools (i.e., efflux/influx rate is high) in an individual, for example, plasma label will be lost in tissues and replaced by unlabeled cholesterol and therefore will not be recovered in fecal sterols (FIG. 9A). This will be interpreted as low % recovery. But this circumstance may in fact represent a good thing with regard to anti-atherosclerotic risk (higher rate of cholesterol mobilization or flush rate from tissues) and should not count against efficiency of the global RCT process in the individual. Any functional measure of labeled plasma cholesterol excretion therefore should preferably correct for the efflux/influx rate across tissues (FIG. 9B). These considerations also apply to methods administering cholesterol label in cells (e.g., macrophages labeled ex vivo)

C. Calculation of a “Global RCT” Parameter.

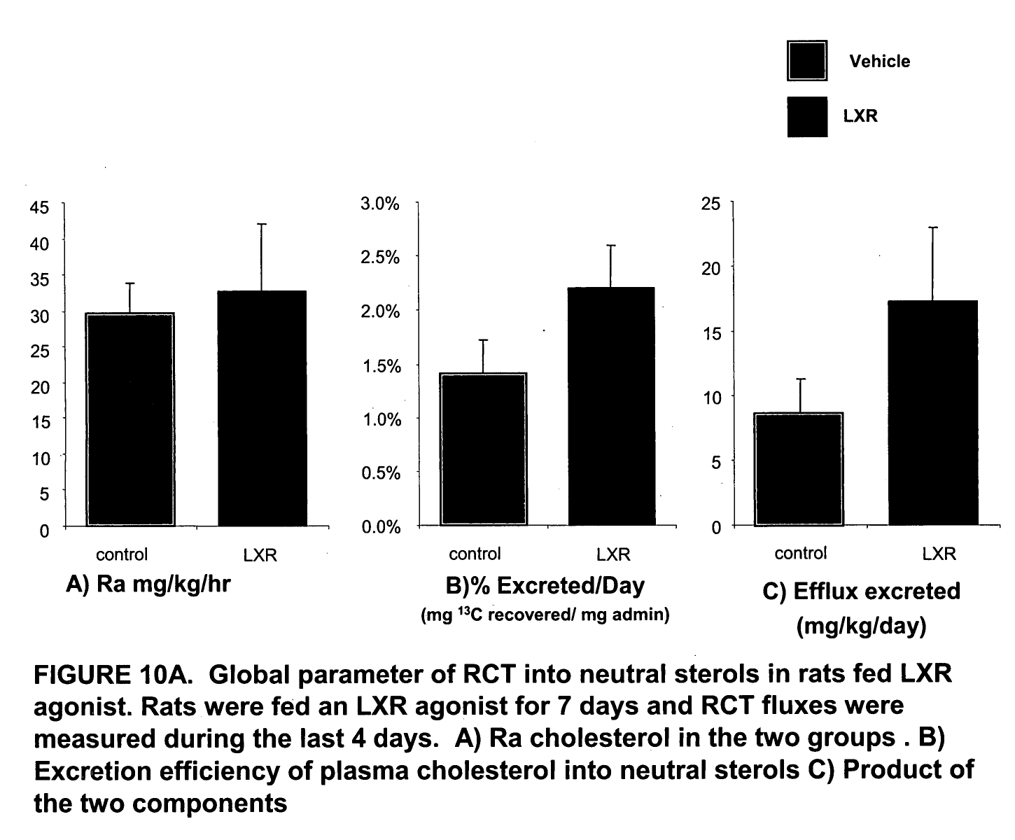

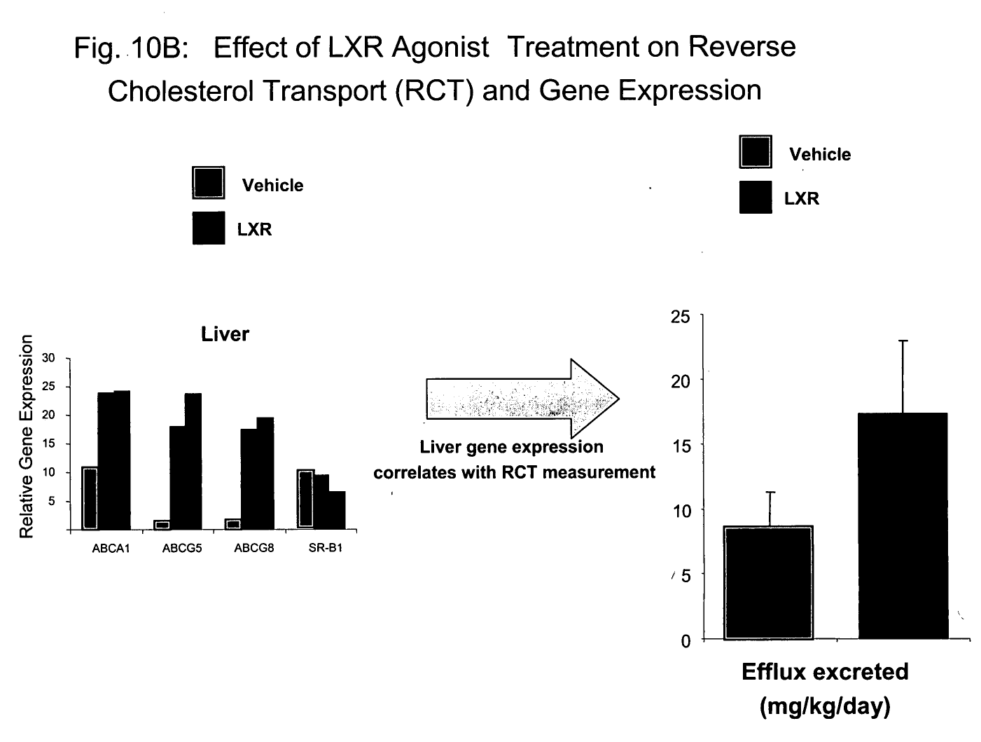

As disclosed herein, it has been discovered that, by combining the efflux/mobilization rate with the efficiency of label recovery in fecal sterols, a “Global RCT” parameter can be measured (FIGS. 9B-C and FIGS. 10-13)). This Global RCT metric provides an integrated measure of the cholesterol flux from tissues to stool in living organisms, including human subjects.

Global RCT flux represents the cholesterol efflux from tissues that ends up excreted as fecal sterols. This can be seen, intuitively, as the # of cholesterol molecules entering the plasma pool/day multiplied times the proportion of plasma cholesterol molecules that are recovered as fecal sterols. It can be understood by those of skill in the art that this metric represents the cholesterol flux rate from tissues through the bloodstream and out of the body—the definition of global RCT.

Accordingly, this parameter has been used extensively to characterize the effects of potential therapeutic agents on global RCT flux in the whole organism (see examples, infra).

D. Division of the Global RCT Parameter into the Two Arms of RCT for Dissecting Locus of Action of Therapeutic Agents that Increase RCT

As disclosed herein, it has also been discovered that it is useful to divide the Global RCT parameter into two component arms (efflux/mobilization and excretion, FIG. 9C and FIGS. 10-13), for the purpose of dissecting the locus of action of therapeutic agents that increase RCT. It can be very useful in drug discovery or subject research to know which aspect of RCT is altered by a drug or disease condition. Examples of this are provided (FIGS. 10-13 and see infra).

IV. Further Embodiments.

The methods described herein may be practiced in a variety of ways depending on the preference of the practitioner. Certain of these embodiments are discussed below, which are not meant to be limiting.

A. Measurement of Efflux Component of RCT

In one embodiment of the invention, the efflux component of RCT is measured in humans or an experimental animal model by intravenous infusion of ¹³C₂-labeled cholesterol in a lipid emulsion at a constant rate, and the isotopic content or isotopic pattern of total blood cholesterol is measured as needed to characterize the isotopic plateau, or conveniently every approximately 1-2 hours for approximately 12-18 hours. Total blood or plasma-free or lipoprotein-associated cholesterol is purified, the isotopic content or isotopic pattern is determined by GC/C-IR/MS or other methods known in the art, and then expressed as atom percent excess of ¹³C. The rate of efflux is then calculated as described, supra.

As disclosed herein, several key discoveries allow the measurement of cholesterol efflux rate from cells in living organisms, including humans: (1) measurement of flux by the dilution method is optimally performed when the “plateau principle” can be exploited. This principle states, in essence, that attainment of an isotopic plateau during a constant infusion of tracer allows calculation of the turnover (rate of appearance, flux) of the endogenous pool simply from the isotopic enrichment or specific activity attained. It had not previously been known whether plasma cholesterol would achieve an isotopic plateau over a reasonable period of time to allow calculation of flux by this approach. As disclosed herein, this circumstance is indeed the case in both human and animal subjects (FIG. 5 and see infra); (2) The efflux rate or rate of appearance of the plasma cholesterol pool was found to be within a range (ca. 10 mg/kg/hr in humans, FIG. 6A) consistent with previous indirect findings for flux and pool size; and (3) Efflux rate is influenced by tissue cholesterol loading (FIG. 7 and see infra), as anticipated.

In another embodiment of the invention, the efflux component of RCT into a particular subclass of plasma lipoproteins (small or large HDL particles, for example) is measured in humans or an experimental animal model by intravenous infusion of ¹³C₂-labeled cholesterol in a lipid emulsion at a constant rate. The isotopic content or isotopic pattern of cholesterol in the plasma lipoprotein particle of interest is measured every approximately 1-2 hours for approximately 12-18 hours. Free cholesterol is purified and the isotopic content or isotopic pattern is determined by GCC-IR-MS or other methods known in the art, and then expressed as atom percent excess of ¹³C. The rate of efflux into the particular subclass of plasma lipoproteins is then calculated as described, supra.

B. Measurement of the Hepatic or Excretory Component of RCT

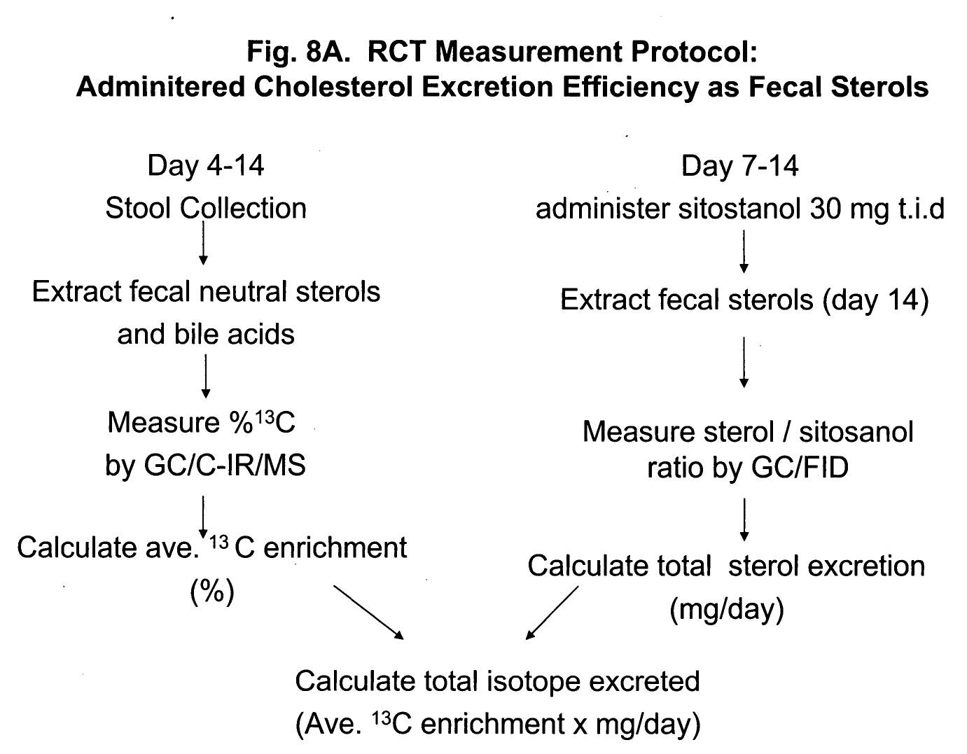

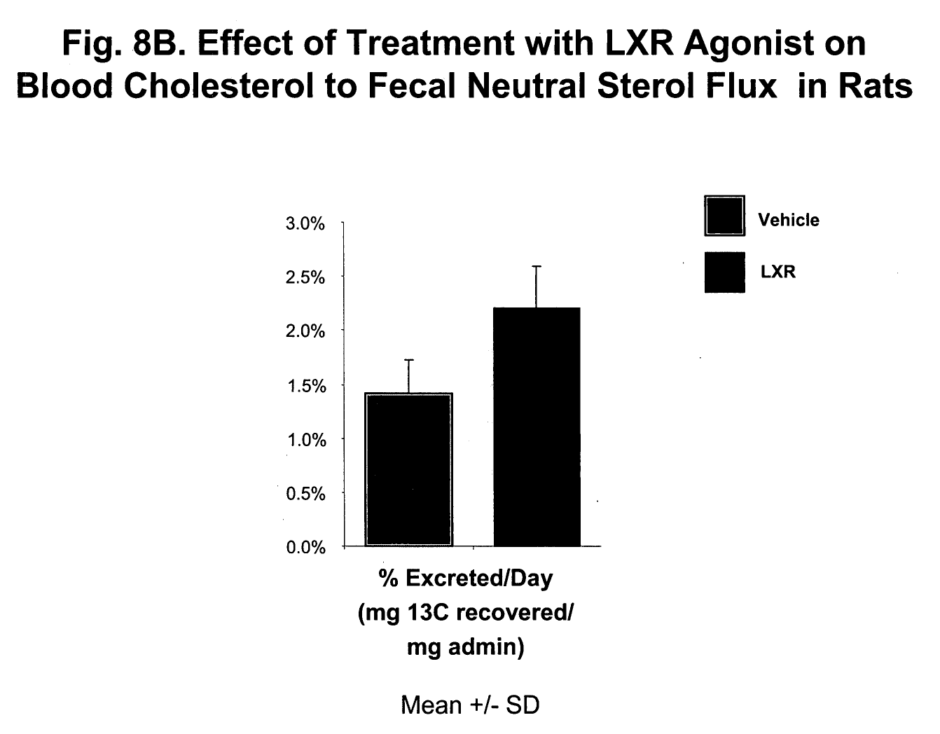

In another embodiment of the invention, the hepatic or excretory component of RCT is measured in humans by administering by the intravenous route ¹³C₂ labeled cholesterol in a lipid emulsion. Biological samples of urine or stool are collected prior to administration of the bolus, and daily thereafter for a period of up to 28 days. Cholic acid and deoxycholic acid, as well as neutral sterols, are purified from the urine or stool samples and then analyzed by GCC-IRMS or other methods known in the art for detecting isotopic content or isotopic pattern (FIG. 8A). The isotopic content or isotopic pattern is expressed as atom percent excess (APE) of ¹³C. The isotopic content or isotopic pattern of cholic acid that is determined prior to the administration of ¹³C₂-labeled cholesterol is used as a baseline value, and the maximum possible APE is estimated based on the amount of ¹³C₂-labeled cholesterol administered to the subject and the weight and body composition of the subject. The fraction of administered ¹³C₂ labeled cholesterol recovered as bile acids, the total conversion rate of administered ¹³C₂ labeled cholesterol to bile acids or neutral sterols, and the fraction of bile acids derived from plasma cholesterol are then calculated as described, supra (FIG. 8B).

C. Measurement of the Hepatic or Excretory Component of RCT with Bile Acid Pool Size Determination.

In yet another embodiment of the invention, the hepatic or excretory component of RCT is measured, for example, in humans, by administering an intravenous bolus of ¹³C₂ labeled cholesterol in a lipid emulsion. Additionally, a known amount of ²H₄-cholic acid is administered orally. Biological samples of urine are collected prior to the administration of the bolus, and daily thereafter for a period of up to 28 days. Biological samples of blood are collected prior to (day 0) and 2, 4, 7, and 14 days after label administration. Cholic acid is purified from blood samples taken between 0 and 48 hours post label administration, and the ²H isotopic content or isotopic pattern of cholic acid is measured by GC/MS or other methods known in the art, and expressed as molar percent excess of the M₄ ion. Cholesterol is purified from all blood samples and the ¹³C isotopic content or isotopic pattern is measured by GCC-IRMS or other methods known in the art and is expressed as APE of ¹³C. Cholic acid is purified from the urine and the ¹³C isotopic content or isotopic pattern is measured by GCC-IRMS or other methods known in the art and is expressed as APE of ¹³C. The ²H isotopic content or isotopic pattern of blood cholic acid is used to calculate the bile acid pool size. The fraction of administered ¹³C₂ labeled cholesterol recovered as bile acids, the total conversion rate of administered ¹³C₂ labeled cholesterol to bile acids, and the fraction of bile acids derived from plasma cholesterol is determined using the ¹³C isotopic content or isotopic pattern of urinary cholic acid, with the day 0 urinary cholic acid ¹³C isotopic content or isotopic pattern used as a baseline value, and the blood cholesterol ¹³C isotopic content or isotopic pattern used to determine the maximum possible ¹³C APE for urinary cholic acid. These values are then combined with mass excretion rates (which are measured directly by simple techniques known in the art) to calculate the rate of blood cholesterol conversion or transport into bile acids and bile sterols during the period of the experiment. This may be expressed as a mass conversion rate (a flux) with units of

$$\frac{cholesterol ( grams ) \text{/} {kg}_{bodyweight}}{day}$$

This quantity represents the flux through the hepatic or excretory component of RCT.

D. Measurement of the Hepatic or Excretory Component of RCT with Bile Acid Pool Size Determination and Quantitation of de Novo Cholesterol Synthesis and its Contribution to Bile Acids.

In yet another embodiment, the hepatic or excretory component of RCT is determined as described in sections IV-C, supra. Additionally, in a preferred embodiment, the subject receives multiple oral doses of approximately 70% deuterated water. The ¹³C APE and M₁ MPE are measured for urinary cholic acid. The ¹³C-APE and M₁ MPE are determined for the urinary bile neutral sterol metabolites. The concentration of deuterated water in blood may also be determined from the blood samples. The contribution of de novo synthesized cholesterol to bile acids and neutral sterols is then determined using the M₁ MPE's, the M₁ MPE's at day 0 (as a baseline) and a maximum possible M₁ MPE's calculated based on the concentration of deuterated water (the isotope-labeled cholesterol precursor) in the blood. These calculations are carried out as described, supra.

The data derived from this experiment can be used to calculate the mass or fraction of bile acids and neutral sterols derived from the hepatic or excretory component of RCT, the mass or fraction of bile acids and neutral sterols derived from de novo synthesized cholesterol, and the mass or fraction of bile acids and neutral sterols which were present in the subject prior to the start of the study, or which are derived from bile acids and neutral sterols present in the subject prior to the start of the study.

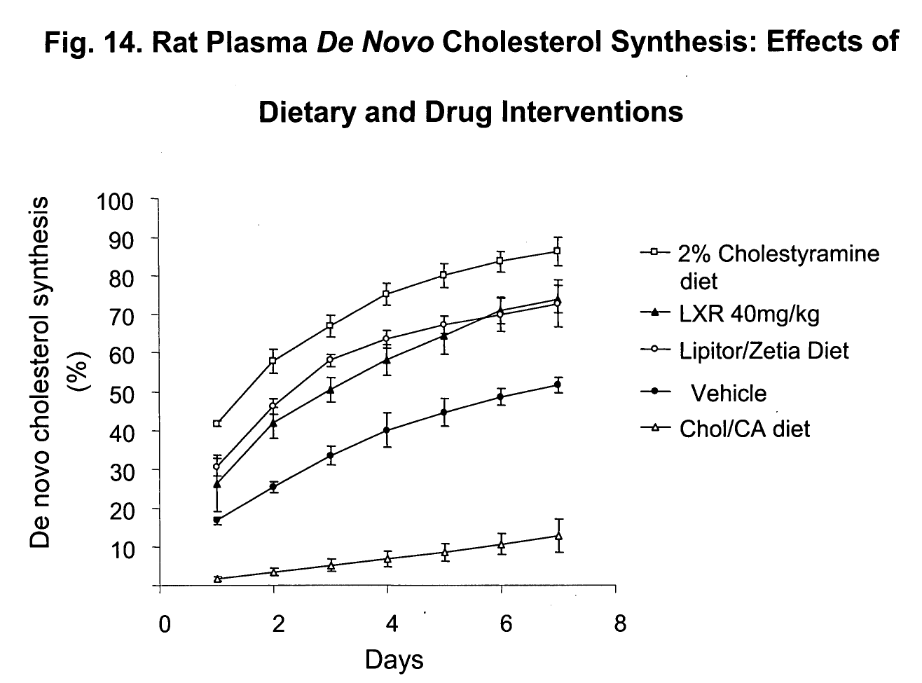

The data derived from this experiment can also be used as complementary evidence for the rate of RCT out of tissues, based on the principle that all cholesterol that exits tissues must be balanced at steady-state by de novo synthesis of cholesterol in the tissue. Thus, evidence for efflux of cholesterol from peripheral tissues and/or excretion of cholesterol from the body may be expected to result in increased rates of de novo synthesis of cholesterol (FIG. 14).

If excretion rates are determined (using skills known in the art), then the rate of excretion of bile acids and neutral sterols from either RCT or DNC can be calculated as well.

E. Simultaneous Measurement of the Efflux Component and Hepatic or Excretory Component of RCT

In yet another embodiment, the efflux and hepatic or excretory components of RCT are measured simultaneously. In this case, the ¹³C₂-labeled cholesterol administered to a subject is administered as a constant intravenous infusion for 12-18 hours. Blood samples are taken every 1-2 hours over the course of the infusion in order to determine the efflux component of RCT. Other elements for the determination of the hepatic or excretory component of RCT are carried out as described, supra (FIGS. 10-13).

F. Measurement of the Plasma Component of RCT

In yet another preferred embodiment of the invention, a portion of the plasma component of RCT (the LCAT/CETP mediated component) is measured. ¹³C₂-cholesterol in complex with HDL is administered as an intravenous bolus to a subject. Blood samples are taken prior to the administration of the bolus, every two hours for up to 24 hours after administration of the bolus, and less frequently thereafter for up to three more days. HDL, VLDL and LDL are isolated from the blood samples and the isotopic content or isotopic pattern of HDL-cholesterol, LDL-cholesterol ester, and VLDL cholesterol ester are determined by GCC-IRMS. The transformation of cholesterol to cholesterol ester and the transfer of cholesterol ester from HDL to LDL or VLDL are determined by this method. The LCAT component of plasma RCT may also be specifically measured by determining the isotopic content or isotopic pattern of HDL-cholesterol ester as well.

G. Cholesterol Transport and Metabolism in Animals.

The above embodiments can be carried out in animals using the methods as described for humans, supra. In one embodiment, the animals are primates, such as monkeys. In another embodiment, the animals are rabbits, guinea pigs or hamsters. In yet another embodiment, the animals are rodents, such as rats or mice.

H. Combinations.

The methods above may be combined in various forms, with different isotopes or isotope-labeled cholesterol or cholesterol-related molecules or cholesterol-related complexes, isotope-labeled bile acids or isotope-labeled cholesterol precursors. Various combinations of such labeled molecules may be used. Additionally, a broad range of routes and modes of administration, types and times of biological sampling, methods of sample processing, and methods of isotopic content analysis, methods of data calculation, methods of data analysis, and methods of data interpretation are contemplated, supra, and may be combined to suit the needs of the practitioner and is within the skill of those in the art.

V. Products of the Invention

Through the practice of the invention a number of products are generated. In particular, molecules of interest containing isotope-labeled components are generated. Such products take on a number of forms.

A. Biological Samples.

The practice of the invention involves the collection of biological samples containing molecules of interest. A portion of the molecules of interest are stable-isotope labeled. The product is a biological sample containing a pool of molecules of interest (e.g., a milligram of deoxycholic acid) some of which are isotopically-labeled.

B. Portions of Biological Samples.

The product may also be the purified, isolated, partially purified, or derivatized or modified (for analysis) pool of molecules of interest (e.g., 500 micrograms of coprostanol isolated from human urine) from a biological sample obtained by practicing the methods of the present invention. The product may be a fraction of a biological sample that is prepared in order to enrich for the molecule of interest (e.g., the lipoprotein fraction of blood), or to remove unwanted molecules that may interfere with analysis.

C. Labeled Molecules of Interest.

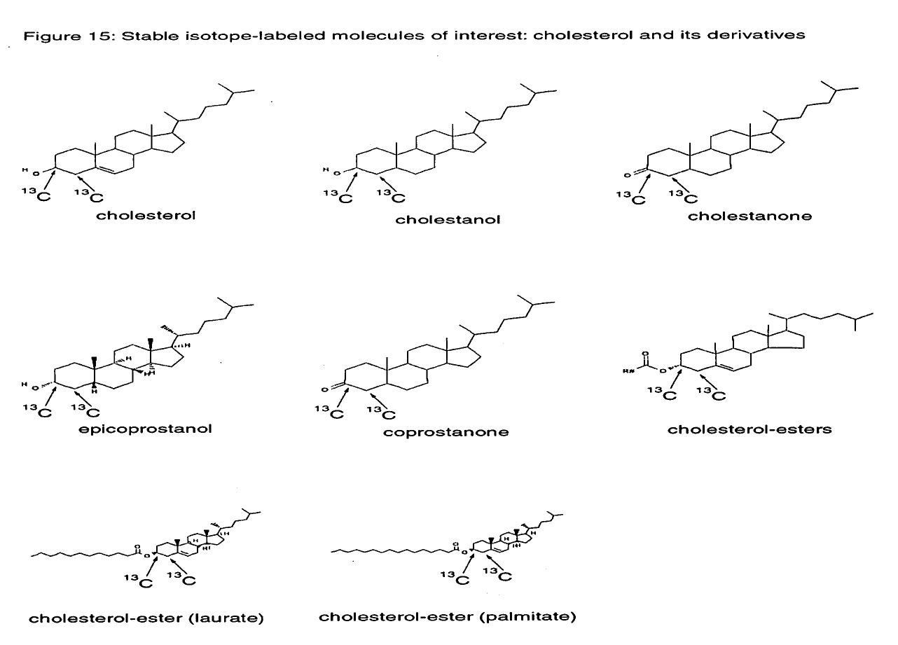

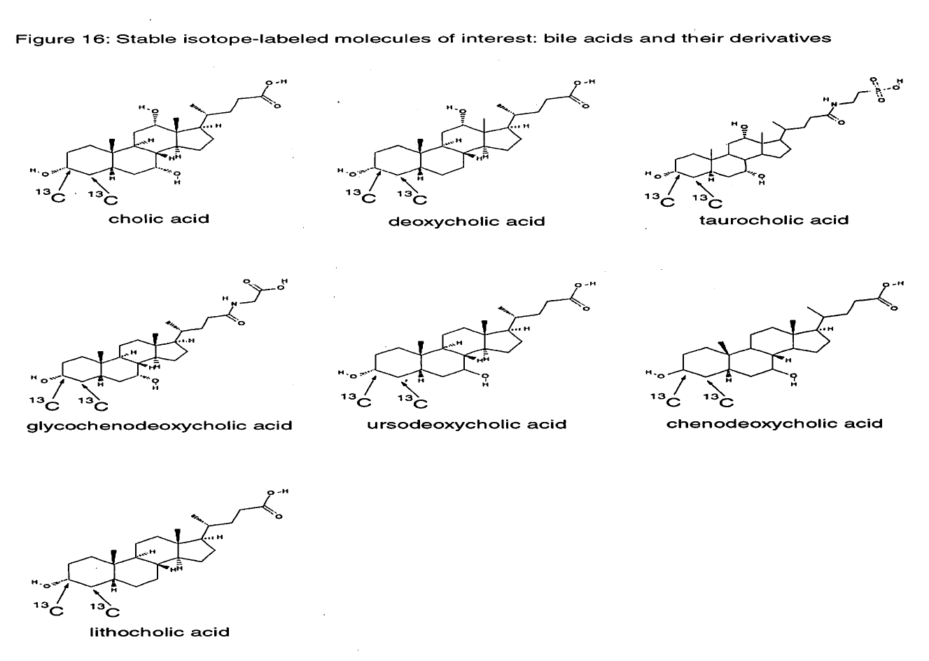

The products generated by this invention also include specific isotope-labeled molecules. A non-limiting list of such molecules is shown in FIGS. 15 and 16, which illustrate a range of molecules of interest and show labeling positions for ¹³C. These particular molecules would derive from the administration of ¹³C₂-labeled cholesterol during the practice of the invention, although the embodiments described herein could generate a broad range of isotope-labeled molecules of interest, including those labeled with other isotopes.

Uses of the Present Invention

The methods of the present invention may be used for a variety of purposes. For example, the methods may be used to determine the rates of the components of RCT in a subject. In turn, the rates may be used to assess the effect of various candidate therapies on atherogenesis and/or atherosclerosis or other cholesterol-related diseases, including coronary heart disease, peripheral vascular disease and cerebral vascular disease.

In one aspect, the methods may be used to determine the effect of a candidate therapy on RCT. After administering the candidate therapy to a subject, the rate of one or more components of RCT in the subject before and after administration of the candidate therapy may be compared. The subject may or may not have atherosclerosis. The effect of the candidate therapy will be determined by the change (e.g., increase, decrease, or no difference) in the rate measured before and after administration of the candidate therapy. Such effects can also be measured by administering the candidate therapy to one group of subjects, and administering placebo or no therapy to the other group, and comparing RCT in the two groups. Similarly, candidate therapies may be administered to a single group, and the efflux component in this group may be compared to historical data on cholesterol efflux. Other types of pre-clinical or clinical study design, known to those of skill in the art, can be employed while practicing the methods of the present invention.

In another aspect of the invention, the methods are employed in a drug discovery, development, and approval (DDA) project or program. Effects on RCT are observed after a living system is exposed to a candidate therapy or combination of candidate therapies. The data generated and analyzed facilitates the DDA decision-making process; i.e., it provides useful information for decision-makers in their decision to continue with further development of a candidate therapy (e.g., if the RCT data appear promising) or to cease efforts, for example, if the RCT data appear unfavorable. By this means, proposed molecular targets can be evaluated for the effects of alterations in their activity, e.g., by inhibition or stimulation, on cholesterol transport, and RCT in particular. The functional importance and role in cholesterol transport, and RCT of proposed molecular targets can thereby be evaluated efficiently in humans and experimental animals.

In yet another aspect of the invention, the methods are used for dose-finding and/or optimization. A candidate therapy may be administered to subjects (animal or human) over a range of doses or dosing schedules, and the optimal dose may then be selected based on dose-response of RCT to the candidate therapy. The methods may further be used to determine what dose is appropriate for different classes of subject, e.g., a subject who is already receiving statin or fibrate therapy, or a subject with a genetic defect in cholesterol metabolism who may require a different dose of a candidate therapy in order to have a beneficial effect on RCT.

In yet another aspect of the invention, the methods are used for formulation development. A candidate therapy may be formulated in a variety of excipients or administered by a variety of routes. The effect on RCT is then used to determine which excipient or route is optimal. For example, a candidate therapy may be found to be more effective at modulating RCT if it is administered twice daily, or if it is administered at mealtime. A candidate therapy may also be found to be more effective if given in a time-release formulation, or it may be found to be more effective if given in a single dose that is rapidly absorbed or it may be found to be more effective if formulated with a particular excipient or excipients or ratios of particular excipients.

In yet another aspect of the invention, the methods allow for the selection of subjects for evaluation of candidate therapies (e.g., in a clinical trial), or for their ability to respond to candidate therapies. Given the range of causes of cholesterol-related disease, it is possible that only particular subjects may respond to a given candidate therapy. In this case, the methods can be used to determine whether or not a subject is appropriate for a clinical trial. For instance, a hypercholesterolemic subject who also has high levels of plasma HDL may not respond favorably to an RCT therapy. Such a subject may be excluded from a clinical trial. Similarly, for candidate therapies that are being used to treat subjects (i.e., candidate therapies that are approved for use in humans—a sub-class of candidate therapies), the methods of the present invention enable the clinician to determine the appropriateness of a given candidate therapy for a given subject or subject.

In yet another aspect of the invention, the methods allow for the skilled artisan to identify, select, and/or characterize the optimal candidate therapy from a group of candidate therapies (e.g., multiple candidate therapies derived from the same lead compound, or multiple candidate therapies that have been partially developed, or multiple candidate therapies from the same compound library). Once identified, selected, and/or characterized, the skilled artisan, based on the information generated by the methods of the present invention, may decide to develop or evaluate the optimal candidate therapy further or to license the candidate therapy to another entity such as a pharmaceutical company or biotechnology company.

In yet another aspect, data generated by the methods of the present invention may be relevant to understanding an underlying molecular pathogenesis, or causation of, one or more cholesterol-related diseases. In another aspect, data generated by the methods of the present invention may shed light on fundamental aspects of the initiation, progression, severity, pathology, aggressiveness, grade, activity, disability, mortality, morbidity, disease sub-classification or other underlying pathogenic or pathologic feature of a cholesterol-related disease of interest.

In yet another aspect, the data generated by the methods of the present invention may provide elucidation on fundamental aspects of the prognosis, survival, morbidity, mortality, stage, therapeutic response, symptomatology, disability or other clinical factor of cholesterol-related disease of interest.

In another aspect, the methods may be used to assess the effect of dietary modification on cholesterol metabolism and transport, including RCT. Similar to what is described above, the effect is determined by the change (e.g., increase, decrease, or no difference) in the rate of one or more components of RCT as determined before and after dietary modification.

In another aspect, the methods may be used to assess the effect of exercise on cholesterol metabolism and transport.

In another aspect, combinations of candidate therapies such as candidate agent administration, dietary modification, and/or exercise may be evaluated by use of the methods of the present invention to assess such combinations of candidate therapies on the rate of one or more components of RCT.

In a further aspect, the invention provides kits for performing the methods of the invention. The kits may be formed to include such components as isotope-labeled cholesterol or cholesterol-related molecules or cholesterol-related complexes, isotope-labeled bile acids, or isotope-labeled cholesterol precursors, or combinations thereof, in varying isotope concentrations and as pre-measured volumes. The kits may be packaged with instructions for use of the kit components and with instructions on how to calculate cholesterol dilutions.

Other kit components, such as tools for administration of the various isotope-labeled components (e.g., measuring cup, needles, syringes, pipettes, IV tubing), may optionally be provided in the kit. Similarly, instruments for obtaining samples from the subject (e.g., specimen cups, needles, syringes) may also be optionally provided.

The following examples are provided to show that the method of the invention may be used to determine the various components of reverse cholesterol transport in humans and in animals. Those skilled in the art will recognize that while specific embodiments have been illustrated and described, they are not intended to limit the invention.

### EXAMPLES

### Example 1

### Measurement of the Efflux Component of RCT in Humans

Introduction

The efflux component of RCT is the first step in the removal of cholesterol from tissues and from the whole organism.

Methods

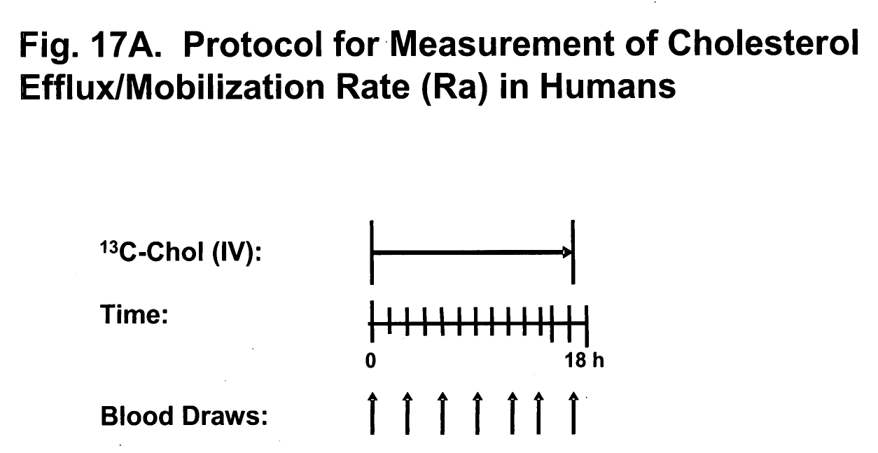

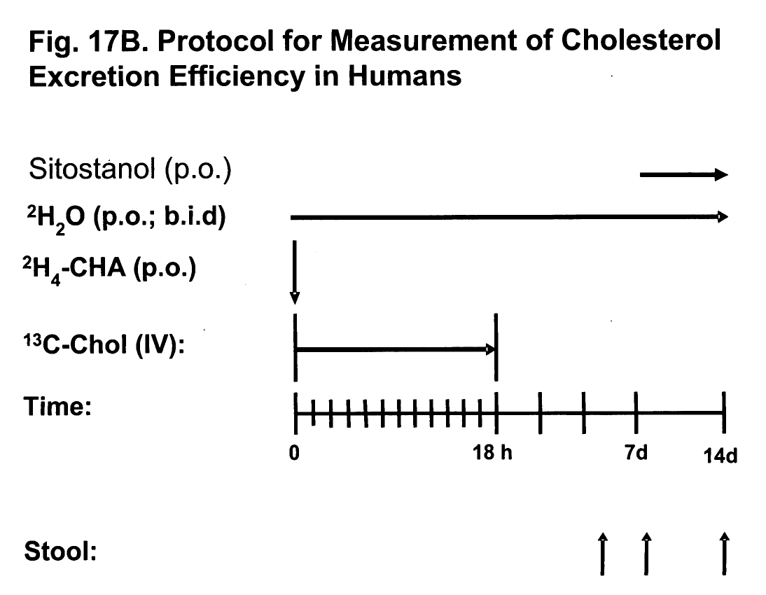

Six healthy human volunteers were administered 200 mgs of 100% ¹³C₂ cholesterol intravenously at a rate of 16.66 mgs/hour for 12 hours (FIG. 17A). 20 to 40 mls of blood were collected from each subject 6 to 8 times over the course of the infusion. Blood was collected immediately prior to infusion for five of the six subjects. Total serum cholesterol was isolated by aqueous organic extraction using methods known in the art. Cholesterol was then derivatized to its acetyl derivative using acetyl chloride, extracted into petroleum ether, dried over sodium sulfate under nitrogen and reconstituted in toluene. It was subsequently injected onto an Agilent model 6890 Gas Chromatograph coupled to a MAT 253 Thermo-Finnigan IRMS, running in combustion mode. Data was compared to a set of standards with a known ¹³C APE in order to determine the APE of each sample.

FIG. 5 shows APE data as a function of infusion time for three of the subjects. In all three subjects shown, ¹³C APE in total serum cholesterol is seen to rise toward a plateau. The plateau value represents the steady state enrichment value that can be used to calculate the dilution rate of labeled cholesterol by endogenous cholesterol efflux. Because not all of the subjects reached steady state enrichment by the end of the experiment, the plateau was determined by fitting the available data to an exponential curve such as the following:
¹³C_{APE}=a(1−e^{(-bt)})

where “a” is the plateau value.

The dilution rate due to endogenous cholesterol (i.e., the rate of appearance of endogenous cholesterol) was calculated using the equation below (and as described, supra):

$$DilutionRate = \frac{InfusionRate ( labeledCholesterol )}{Enrichment ( LabeledCholesterol )} - InfusionRate ( LabeledCholesterol )$$

For example, subject 3 had a plateau ¹³C APE value of 0.00138%, and received cholesterol at a rate of 16.66 mgs/hour. The infused cholesterol was labeled on 2 of 27 carbons, and so the scaling factor for the measured ¹³C APE is 27/2 (This can conceptually be viewed as converting the ¹³C APE into a cholesterol M₂ MPE—such scaling factors are known in the art). The dilution rate equation then becomes:

$$DilutionRate = \frac{16.66 \frac{mg}{hr}}{0.01863} - 16.66 \frac{mg}{hr} = 878 \frac{mg}{hr}$$

Results and Significance

Rates of cholesterol efflux were determined as described for all six subjects:

| Subject ID: | Rate of efflux: |
|---|---|
| 001 | 891 mg/hr |
| 002 | 640 mg/hr |
| 003 | 647 mg/hr |
| 004 | 878 mg/hr |
| 005 | 979 mg/hr |
| 006 | 959 mg/hr |

These rates represent the first component of RCT in each subject.

An additional 15 subjects were studied, demonstrating the inter-individual variability of the measurement for both k and Ra calculated from the rise to plateau fit (FIG. 6A).

This experiment illustrates that the efflux component of RCT can be measured in humans. The technique is accurate and relatively rapid. Measurement of the efflux component of RCT may be used to assess the action of various candidate therapies on RCT, or to evaluate the utility of various candidate therapies for the prevention or treatment of atherogenesis, atherosclerosis, or arteriosclerosis. For instance, the efflux component of RCT in the subject before and after administration of the drug agent can be compared. The subject may or may not have atherosclerosis. The effect of the candidate therapy will be determined by the change (e.g., increase, decrease, or no difference) in the efflux component of RCT as measured before and after administration of the candidate therapy. Such differences can also be measured by administering candidate therapy to one group of subjects, and administering placebo or no therapy to the other group, and comparing the efflux component of RCT in the two groups. Similarly, candidate therapies may be administered to a single group, and the efflux component in this group may be compared to historical data on cholesterol efflux.

Candidate therapies identified by this method have significant commercial value.

### Example 2

### Measurement of the Plasma Component of RCT in Humans; Formation of Cholesterol Ester

Introduction

The metabolism of cholesterol and its transfer between various carrier molecules is an important component of RCT. A major current molecular target for the pharmaceutical industry is CETP (see FIG. 1). Similarly, modulating the activity of LCAT is a legitimate goal for developing novel treatments, as LCAT inhibition may modulate the movement of cholesterol through the RCT pathway in a beneficial manner. The present invention provides tools for the measurement of the plasma component of RCT, including methods for the in vivo measurement of LCAT action, CETP action, or both.

Methods

Two healthy volunteers were administered an intravenous bolus of 100 or 200 mgs of ¹³C₂-cholesterol suspended in a lipid emulsion. Blood samples of 20 to 40 mls were collected 3 to 4 times in the first 24 hours after the administration of label, and twice thereafter, around 5 and 10 days post-label administration. Cholesterol was isolated from each blood sample by centrifugation followed by methanol/chloroform extraction and then thin layer chromatography (TLC). Cholesterol-ester was isolated by the same techniques, but using a different TLC method Both cholesterol and cholesterol ester were converted to their acetyl chloride derivatives as described, supra. ¹³C APE was determined by GCC-IRMS for cholesterol and cholesterol ester from each sample, as described, supra.

Results and Significance

The ¹³C APE's of cholesterol and cholesterol ester were plotted versus time for both subjects (data not shown). The conversion of cholesterol to cholesterol ester occurs over the course of twelve hours. These data give a direct indication of the activity of LCAT in vivo, and show that this part of the plasma component of RCT occurs rapidly. Like the efflux component of RCT, measurement of either a part or all of the plasma components of RCT, especially in the context of the development of candidate therapies, could yield valuable information about candidate therapy activity in vivo and about drug efficacy.

This experiment illustrates that the plasma component of RCT can be measured in humans. Measurement of the plasma component of RCT may be used to assess the action of various candidate therapies on RCT or cholesterol transport, including CETP inhibitors, LCAT inhibitors, or other such candidate therapies. As with the other components of RCT, the identification of candidate therapies that can modulate the plasma component of RCT would be of significant commercial value.

### Example 3

### Measurement of the Efflux Component of RCT in Rats

Introduction

The efflux component of RCT is the first component of RCT, and is a process that may be targeted by candidate therapies. Any candidate therapy must be tested in animals as part of the process of development. Example 1 demonstrated that the efflux component could be measured in humans. This example shows the same experiment carried out in rats.

Methods

The methods are the same as those discussed in Example 1, supra, except that only 1.2 mgs of ¹³C₂-cholesterol were administered via intravenous infusion (100 micrograms/hour), and only 100 microliters of blood were collected at each time point. Additionally, four groups of animals were studied—rats fed standard rat chow; rats fed a high cholesterol diet; rats fed a high cholesterol diet plus cholic acid; and rats fed the high cholesterol plus cholic acid diet for 14 days and then returned to normal chow for 4 days. In this case, the animal model of disease is diet induced hyper-cholesterolemia the effect of the cholic acid on increasing plasma cholesterol, followed by a relatively rapid return to nomal cholesterol levels on switching to chow. FIG. 7 demonstrates the effect of cholesterol cholic acid feeding (cholesterol loading), and subsequent return to normal show (cholesterol unloading) on the efflux of cholesterol into plasma.

Results and Significance

FIG. 7 shows the average rates of efflux for each treatment group. The cholesterol cholic acid fed animals have the highest efflux rate, indicating that the efflux component of RCT may be upregulated in an attempt to compensate for the effects of the cholesterol loading diet cholesterol metabolism. On returning to chow diet the Ra is lower but not yet normalized indicating persistent efflux of cholesterol from the rat, consistent with the notion that the cholesterol cholic acid diet loaded tissues with cholesterol which is still being cleared. The data herein successfully demonstrate the testing of an intervention in a preclinical (rodent) model.

### Example 4

### Measurement of Cholesterol Excretion RCT in Rats; Measurement of Transport of Cholesterol into Bile and Conversion of Cholesterol into Bile Acids

Introduction

The hepatic component of RCT is relevant to the study of RCT in that it represents the last component of RCT and the point at which cholesterol actually leaves the body. Ideally, measurement of the hepatic component of RCT will, among other things, allow for the determination of the source of and rate of synthesis of all components of the bile, including neutral sterols (e.g., bile cholesterol derived from RCT or from hepatic de novo cholesterol synthesis) as well as bile acids (e.g., deoxycholate derived from RCT cholesterol or hepatic de novo synthesized cholesterol).

Methods

Label Administration and Biological Sampling

Blood and stool samples are collected prior to the administration of any stable-isotope label (referred to as “day 0”).

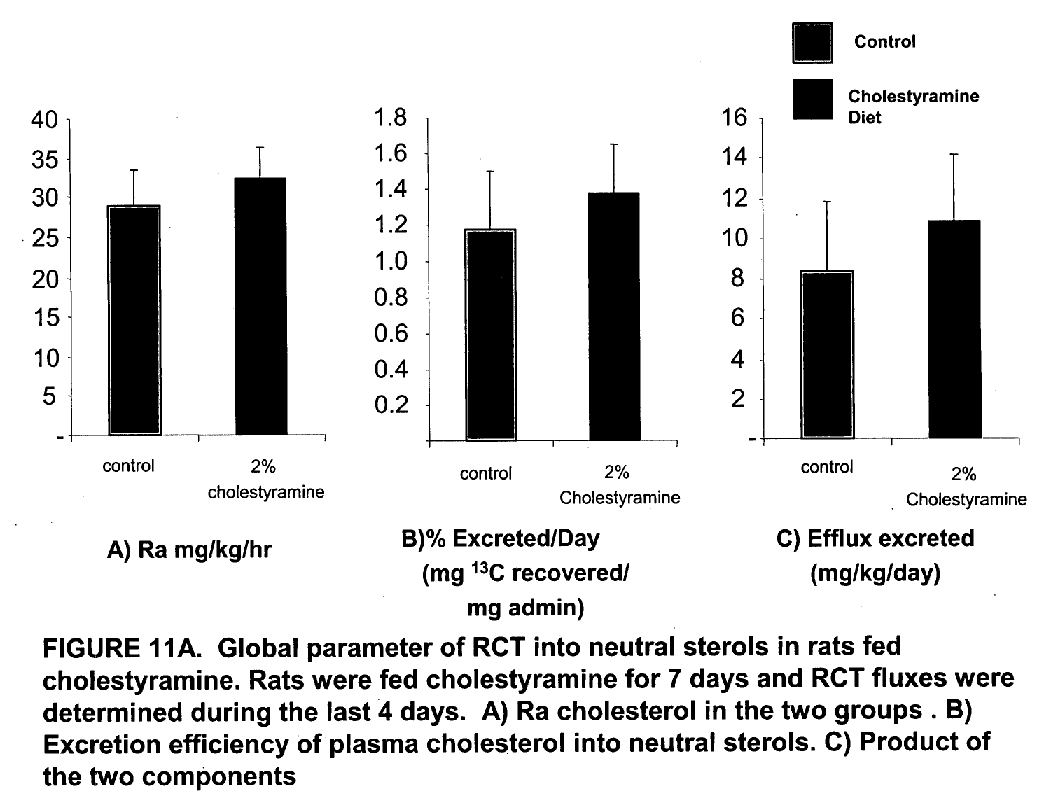

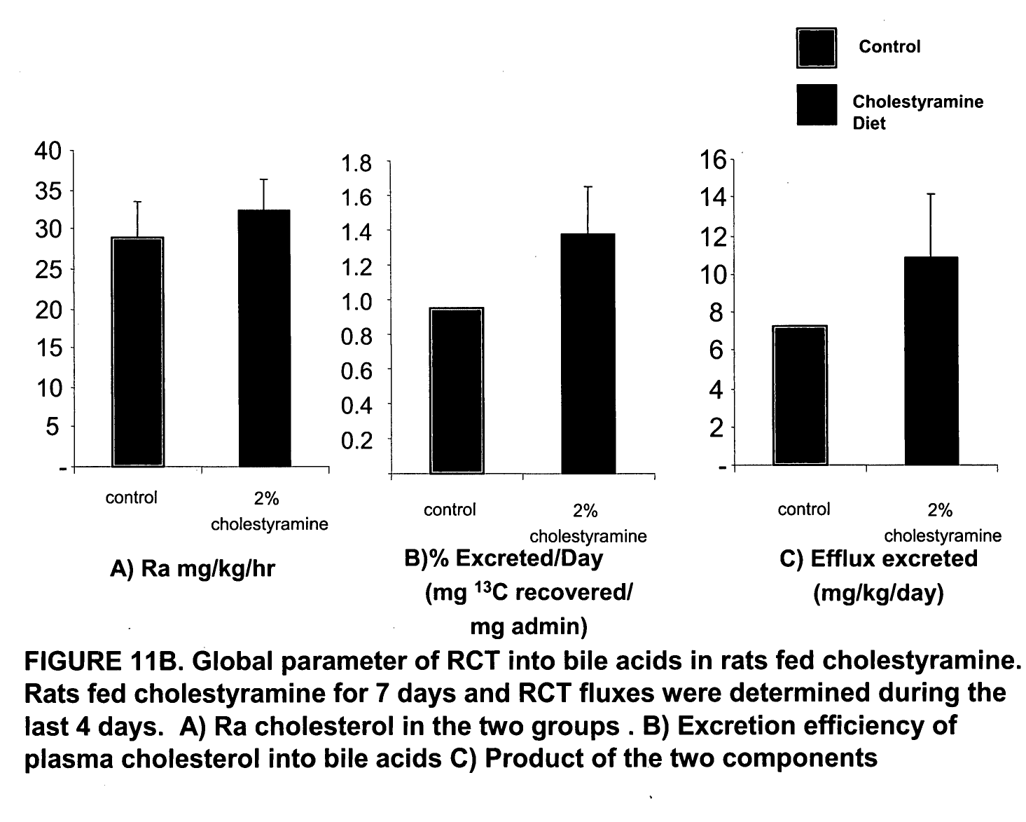

Rats were administered an IP bolus of 100% deuterated water in order to reach a body water with an isotopic content of approximately 5% excess deuterium. Rats were subsequently given 8% excess deuterated water in drinking water to maintain a steady state level of body water deuterium. The incorporation of deuterium from deuterated water into hepatic de novo synthesized cholesterol allows for the determination of the portion of bile cholesterol or bile acids derived from de novo synthesized cholesterol (FIG. 11B).

Rats were also administered an intravenous bolus of 1.2 mgs of ¹³C₂ cholesterol in a lipid emulsion at the same time as the deuterated water bolus.

Blood samples were taken daily over the six days following the bolus doses. Stool samples were collected daily for six days following the bolus doses.

Determination of Sterol Excretion Rate.

Techniques known in the art were employed to determine the mass of each bile component (cholesterol, coprostanol, and deoxycholic acid) excreted each day. Alternatively, the mass values are also obtained from historical data or from the literature if the strain of rat of interest has been studied previously under similar conditions.

Isotopic Content or Isotopic Pattern Determination: Measurements for de Novo Cholesterol Contribution.

A variety of isotopic content or isotopic pattern measurements were made. The concentration of ²H₂O was measured in blood samples taken on days 1 through 6 by reacting plasma with calcium carbide an analyzing the resulting acetylene gas using a Monitor series 3000 cycloidal mass spectrometer. This data provides the basis for the calculation of the maximum possible MPE for the molecules of interest.

Cholesterol, coprostanol, and deoxycholate were purified from stool samples from days 1 through 6, by incubating the stool overnight in a sodium hydroxide solution, performing a hexane extraction to isolate the neutral sterols, neutralizing the remaining aqueous phase with hydrochloric acid, and then extracting with ethyl acetate to isolate the bile acids. The MPE's of the M₁ isotope of cholesterol, coprostanol, and deoxycholic acid were then determined by GC/MS. When combined with the plasma deuterated water concentration, these measurements allow for the determination of the fraction of each molecule of interest derived from hepatic de novo synthesized cholesterol (FIG. 14) and correlation with other metrics of RCT and tissue cholesterol balance.

Isotopic Content or Isotopic Pattern Determination: Measurements for Hepatic RCT Contribution.

A variety of isotopic content or isotopic pattern measurements were made. Cholesterol was purified from the blood samples taken on days 0 through 6 and analyzed for ¹³C APE by GCC-IRMS as described, supra. The day 0 measurement provides the baseline measurement for the ¹³C APE calculation, as described, supra. The ¹³C APE of blood cholesterol was used to calculate the maximum possible APE that can be expected in stool cholesterol, coprostanol, or deoxycholate.

Coprostanol, cholesterol, and deoxycholate were purified from the stool samples taken on days 0 through 6. The molecules were subsequently analyzed by GCC-IRMS for ¹³C APE. When combined with the ¹³C APE of blood cholesterol, these measurements allow for the determination of the fraction of each molecule of interest derived from RCT.

Calculation of Fractional Contributions.

For de novo synthesized cholesterol, the maximum possible MPE of the M₁ isotopomer is determined by multiplying the amount of excess deuterated water in plasma by a scalar coefficient determined by MIDA calculations, or by historical data. Observed MPE EM₁ enrichments of stool cholesterol, coprostanol, and deoxycholic acid were divided by this maximum possible value in order to yield the fraction of each that is derived from hepatic de novo synthesized cholesterol.

For RCT, the ¹³C APE observed in stool cholesterol, coprostanol, and deoxycholic acid was divided by the ¹³C APE observed in plasma cholesterol averaged over an appropriate sampling period (determined based on the frequency of sample collection) in order to get the fraction of each molecule derived from RCT. In this case, the averaging period was 24 hours. For instance, the ¹³C APE for stool cholesterol, coprostanol, and deoxycholic acid from the day 2 stool sample was divided by the average of the day 2 and day 1 ¹³C APE values of blood cholesterol. This time averaging ensures that the maximum possible APE used to calculate fractional contributions reflects the blood cholesterol concentration over the entire period during which the sampled molecules of interest were being synthesized. In theory, in the case of the rat, the ideal averaging period would be averaging over the period of time it takes for the contents of 2 stool pellets (the biological sample size) to be excreted. A skilled practitioner can see, however, that the importance of averaging decreases when the ¹³C APE of blood cholesterol approaches a steady state with respect to sampling frequency (as is observed from day 3 onward, FIG. 8). In these situations, the actual ¹³C APE from blood cholesterol from a single sample can be used as the maximal value.

Results and Significance

The fractional contributions of each cholesterol source (RCT or de novo synthesis) to each molecule of interest (cholesterol, coprostanol, and deoxycholic acid) were multiplied by the excretion rates of each molecule in order to get the mass excreted each day from each source.

The methods of the present invention have extensive preclinical uses for the discovery and development of candidate therapies. Understanding the contributions of de novo synthesis and RCT to bile excretion is critical to such an analysis—simply determining the rate of secretion of any or all bile components is not sufficient. For instance, a therapy that increased the excretion rate of bile might be doing so by increasing RCT, but it may also be doing so by increasing hepatic de novo cholesterol synthesis, in which case the treatment would not be effective at increasing RCT (i.e., increasing the elimination of cholesterol from the body via RCT) and decreasing the risk or incidence of atherosclerosis. Alternatively, a candidate therapy that increases the rate of bile secretion, but does so by an increase in hepatic cholesterol synthesis to a level where RCT is actually decreased is an undesirable mechanism of action and may in fact do more harm then no treatment at all. The methods of the present invention allow one to distinguish between candidate therapies that increase RCT versus those candidate therapies that may increase bile secretion but actually decrease RCT. The methods of the present invention can also be used to identify optimal doses.

Measurement of the hepatic or excretory component or RCT may also be used to evaluate the effect of candidate therapies in various animal models. For instance, the hepatic arm of RCT before and after administration of the drug agent can be compared. The effect of the candidate therapy will be determined by the change (e.g., increase, decrease, or no difference) in hepatic RCT as measured before and after administration of the candidate therapy. Dose ranges or effective doses, the nature of the dose response curve, and other measures of the mechanism and mode of action of a candidate therapy, with respect to RCT-related action, can also be measured in this manner.

In another variation, the methods may be used to assess the effect of dietary modification on the hepatic component of RCT. Similar to that described above, the effect is determined by the change (e.g., increase, decrease, or no difference) in hepatic RCT as determined before and after dietary modification.

Such differences can also be measured by administering candidate therapy to one group of animals, and administering placebo or no therapy to the other group, and comparing the hepatic component of RCT in the two groups.

### Example 5

### Measurement of “Global Cholesterol RCT” Parameter in Rats

A refinement of the approach outlined in Example 1, involves calculating the Ra of plasma cholesterol and combining that measurement with the fractional excretion of administered cholesterol as described in Example 4.

Method

The recovery of plasma cholesterol in stool is defined as the proportion of administered labeled cholesterol that was excreted in fecal neutral sterols. It is expressed as % label recovered from day 1-4 following infusion. It is calculated from: % ¹³C enrichment in fecal neutral sterols, the mg/day excretion of neutral sterols and divided by total mg 1 ¹³C cholesterol administered.

$$\frac{\% {}^{13} C enrichment \times mg excreted}{{}^{13} C cholesterol administered} = \% label recovered$$

The same calculation is made for the recovery of plasma cholesterol into bile acids excreted in stool. Rats were treated with common and investigational cholesterol lowering agents, cholesterolamine, ezetimibe, LXR agonist (TO-901317). The Ra of cholesterol is calculated as described, the recovery of administered cholesterol in fecal sterol or bile acids are calculated as described.

Results and Significance

Shown in FIG. 11 are the changes observed in RCT flux into neutral sterols (FIG. 11A) and bile acids (FIG. 11B) treated with cholestyramine. Cholestyramine is known to selectively inhibit bile acid absorption but not neutral sterol absorbtion. This is reflected in greater increase in the flux of plasma cholesterol into fecal bile acids than that observed in neutral sterols.

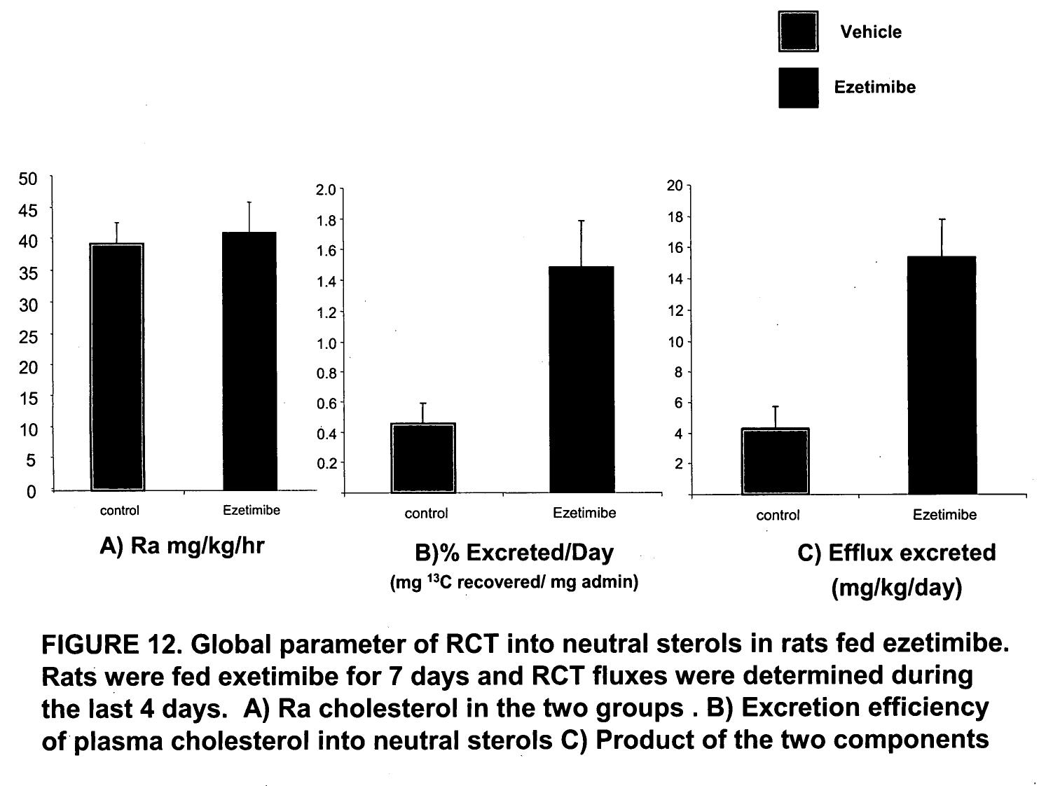

Shown in FIG. 12, is the effect of ezetimibe on RCT. The increase in plasma flux to neutral sterols is consistent with the known mechanism of action of ezetimibe to inhibit intestinal cholesterol absorption, including the re-absorption of endogenous cholesterol secreted from the liver to the bile and into the interstine.

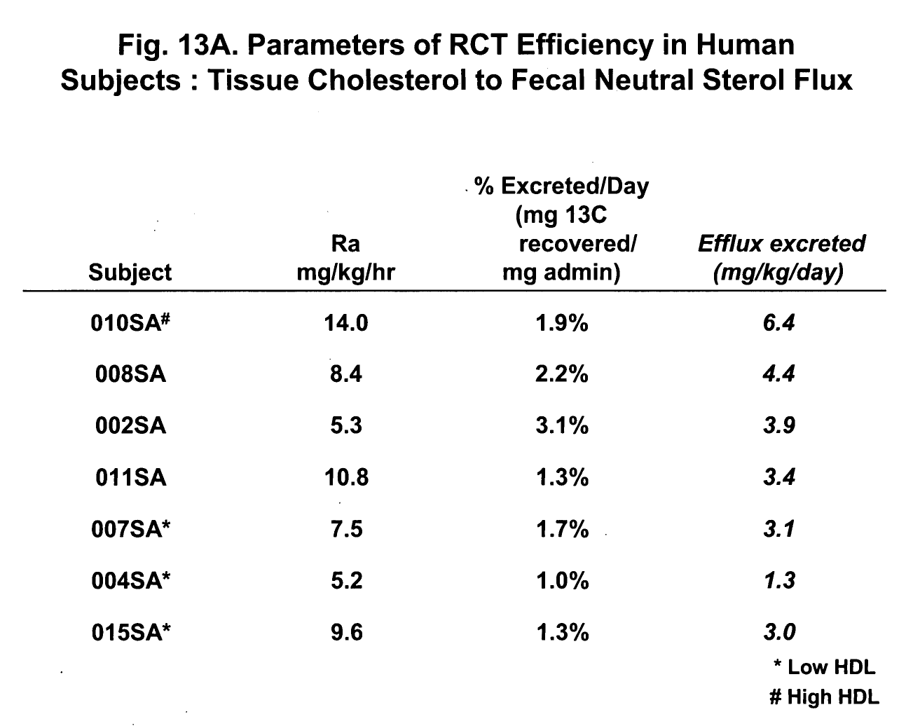

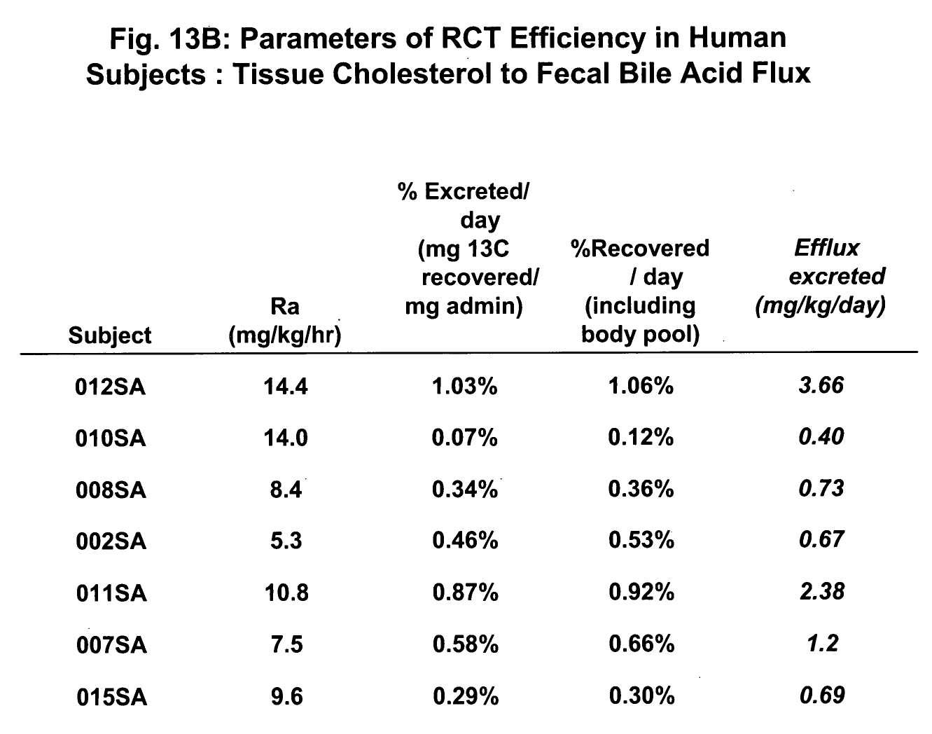

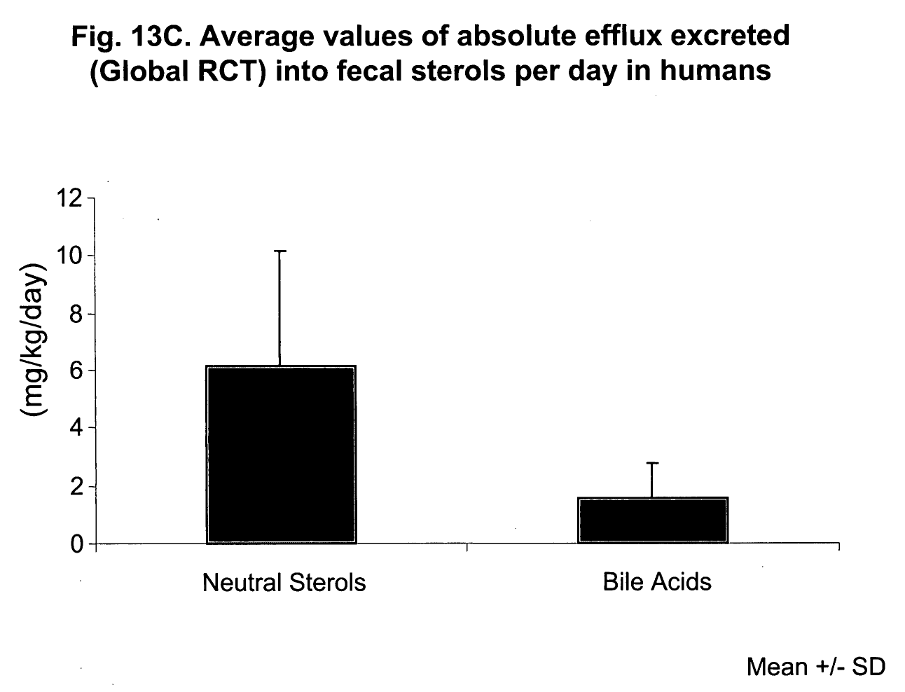

Shown in FIG. 13, the effect of an LXR agonist on excretion of plasma cholesterol into neutral sterols and the global parameter of RCT. LXR agonists have been shown to reverse atherosclerosis in mouse models. Consistent with this effect is the observed increase in RCT. Correlation with gene expression (FIG. 13B) also supports the strategy of validating therapeutic targets for drug discovery and development by correlation with measured RCT fluxes.

These results demonstrate the utility of describing the effects of pharmaceutical interventions on RCT and illustrate how they might be used to rank or identify improved activity on the pathway.

### Example 6

### Measurement of the Hepatic Component of RCT in Humans; de Novo Synthesis of Bile Acids, Measurement of Bile Acid Pool Size, Measurement of Conversion of Plasma Cholesterol to Bile Acids

Introduction

The hepatic or excretory component of RCT is relevant to the study of RCT in that it represents a component of RCT at which cholesterol actually leaves the body. Measuring the hepatic or excretory component of RCT in humans can serve a variety of purposes, some of which are described, supra. The evaluation of candidate therapies in human subjects by measuring the hepatic or excretory arm of RCT is an exemplary use of the present invention. The use of such data to justify, plan, or cancel a clinical trial, or to support a regulatory filing for the continued development or approval of a candidate therapy is also an exemplary use of the invention.

Ideally, measurement of the hepatic or excretory component of RCT will, among other things, allow for the determination of the source of and rate of synthesis of all components of the bile, including neutral sterols (e.g., bile cholesterol derived from RCT or from hepatic de novo cholesterol synthesis) as well as bile acids (e.g., deoxycholate derived from RCT cholesterol or hepatic de novo synthesized cholesterol).

Methods

Label Administration and Biological Sampling

Blood, stool, and urine samples were collected in human subjects and in rats prior to the administration of any stable-isotope label (referred to as “day 0”).

Subjects were administered multiple doses of deuterated water in order to reach a body water enrichment of ˜1% excess deuterium. Subjects were also administered an intravenous bolus of ¹³C₂-cholesterol and receive 50 mgs of ²H₄ cholic acid orally. The incorporation of deuterium from deuterated water into hepatic de novo synthesized cholesterol allows for the determination of the portion of bile cholesterol or bile acids derived from de novo synthesized cholesterol. The appearance of ¹³C in bile acids and bile cholesterol allows for the determination of the portion of these molecules derived from blood cholesterol (i.e., RCT). The dilution of ²H₄ cholic acid allows for the determination of the bile acid pool size.

Multiple biological samples were collected. Stool was collected daily for up to 7 days post label administration in rats and at days 4, 7 and 14 in humans. Blood was collected periodically for up 10 days (1-2 ml). Urine was collected periodically for up to ten days in humans (samples of at least 50 mls).

Different subjects may be administered different labeled molecules or be subjected to different biological sampling regimens. Data from subjects or groups of subjects may be combined with data from other similar subjects in order to form a complete picture of RCT in a population of subjects (e.g., healthy adults, those with hypercholesterolemia, etc.). In one embodiment, all measurements are made in rats on different drug treatment regimens.

Isotopic Content or Isotopic Pattern Determination: Measurements for de Novo Cholesterol Contribution, Calculation of Fractional Contribution of DNC to Bile Components.

The concentration of ²H₂O was measured in blood samples taken on days 0 through 7 by reacting plasma with calcium carbide an analyzing the resulting acetylene gas using a Monitor series 3000 cycloidal mass spectrometer. This data provides the basis for the calculation of the maximum possible MPE for de novo synthesized cholesterol.

Cholesterol and cholic acid were purified from stool samples from days 4 through 7, and cholic acid was purified from urine from days 4 through 7. These molecules are further processed (as described, supra) and then analyzed by GC/MS. The M₁ MPE's were calculated as described above, using a historical reference value for baseline. The maximum possible MPE was calculated from the body water values measured in the blood, using historical data on the relationship between deuterated water and cholesterol synthesis.

The fraction of urinary cholic acid, stool cholic acid, or stool cholesterol that is derived from de novo synthesized hepatic cholesterol was calculated by division of the observed M₁ MPE for each molecule of interest by the maximum possible M₁ MPE calculated from the blood deuterium value.

Isotopic Content or Isotopic Pattern Determination: Measurements for Hepatic RCT Contribution, Calculation of Fractional Contribution of RCT to Bile Components.

For RCT, the ¹³C APE observed in urinary cholic acid is divided by the ¹³C APE observed in plasma cholesterol averaged over an appropriate sampling period (determined based on the frequency of sample collection), as one way to get the fraction of each molecule derived from RCT. In the case of humans, the ¹³C APE of plasma cholesterol is known, from historical data, to be constant at around 0.02 during the time period that samples were collected. Data from two samples (2 week and 3 week) from two healthy subjects are shown in FIG. 12. The calculations indicate that approximately 81% of bile acids derive from RCT in subject 1 and 51% of bile acids derive from RCT in subject 2. These differences may represent clinically relevant differences in RCT, and may indicate a risk for atherogenesis or atherosclerosis in subject 2, who removes less cholesterol in the bile.

Isotopic Content or Isotopic Pattern Determination: Measurements for the Determination of Bile Acid Pool Size, Calculation of the Bile Acid Pool Size.

Cholic acid was isolated from the blood samples taken during the ten day study period. Cholic acid was then purified and analyzed by GC/MS, and the MPE of the M₄ ion was determined, using historical data for a baseline value. The dilution of cholic acid was then calculated based on the amount of cholic acid administered. The bile acid pool size was calculated as using equations known in the art, in particular, those found in Measurement of parameters of cholic acid kinetics in plasma using a microscale stable isotope dilution technique: application to rodents and humans, Hulzebos et al, J. of Lipid Research, volume 42, 2001, pp 1923-1929.

Use of Urinary Sterols as a Proxy for Bile Sterols.

The isotopic content or isotopic pattern of bile acids from urine and metabolites of cholesterol derived from the action of intestinal microbes on bile cholesterol from urine were measured. In this case, the isotopic content or isotopic pattern of bile cholesterol and bile acids can be determined from a urine sample. The details of this technique are described, supra, in section II-C. ²H isotopic content or isotopic pattern measurements of stool and urinary coprostanol in a subject receiving labeled water show that the two are in equilibrium. The isotopic content or isotopic pattern of urinary coprostanol was measured in order to determine the isotopic content of bile cholesterol in stool. Various treatment regimens altered de novo cholesterol synthesis in the rats so studied (FIG. 14).

### Example 7

### Measurement of “Global Cholesterol RCT” Parameter in Humans

A refinement of the approach outlined in Example 6, involves calculating the Ra of plasma cholesterol and combining that measurement with the fractional excretion of administered cholesterol as described in Examples 4 and 5.

Methods

Isotopes are administered as described in Example 6. Oral sitostanol was administered 3 times daily and its recovery in stool samples used to determine the absolute fecal neutral and bile acid excretion rate using methods known in the art.

Results

RCT is shown for seven subjects studied illustrating the interindividual variability between subjects. Additionally subjects with low (<40 mg/dl) and high (>60 mg/dl) plasma HDL cholesterol concentrations are identified. Lowest RCT values are seen in subjects with low HDL (indicated by an * in FIGS. 13A and 13B) and highest are seen with in the subject with highest HDL (indicated by an #).

This experiment illustrates that RCT fluxes into neutral sterols and bile acids can be measured effectively in humans. Furthermore there is an indication that plasma HDL levels may relate to the RCT flux parameter, particularly the global RCT flux parameter.

I claim:

1. A method for determining the molecular flux rate of the hepatic or excretory component of reverse cholesterol transport (RCT) in a living system, said method comprising:
a) administering an isotopically labeled cholesterol molecule or isotopically labeled cholesterol-related molecule to the living system at a known or measurable rate;
b) obtaining a sample from said living system wherein said sample comprises one or more isotopically labeled cholesterol molecules, bile acids or excreted neutral sterols from the living system;
c) measuring isotopic content, isotopic pattern, rate of change of isotopic content, or isotopic pattern of the isotopically labeled cholesterol molecules, bile acids or excreted neutral sterols; and
d) calculating the rate of incorporation or transfer of the isotopically labeled cholesterol molecule or isotopically labeled cholesterol-related molecule into said cholesterol molecules, bile acids or excreted neutral sterols to determine the molecular flux rate of the hepatic or excretory component of RCT in the living system.

2. The method of claim 1 wherein said sample is a stool, urine or blood sample.

3. The method of claim 2 wherein said sample is a stool sample and the isotopic content of labeled neutral sterols and bile acids are measured.

4. The method of claim 1, wherein the isotope label of the isotopically labeled cholesterol molecule or isotopically labeled cholesterol-related molecule is ²H, ³H, ¹³C, ¹⁴C, or ¹⁸O.

5. The method of claim 1 wherein said living system is a human or a rodent.

6. The method of claim 1 wherein ¹³C₂ labeled cholesterol in a lipid emulsion is administered intravenously to said living system.

7. The method of claim 1 further including measuring the total amount of bile acids in said living system by:
i) administering a known amount of isotopically labeled bile acid to said living system;
ii) determining the isotopic content or rate of change in isotopic content of bile acid in said living system after a period of time; and
iii) determining the amount of dilution of the isotope labeled bile acid to measure the total amount of bile acids in said living system.

8. The method of claim 7, wherein the labeled bile acids are chosen from cholic acid, chenodeoxycholic acid, deoxycholic acid and lithocholic acid.

9. The method of claim 8 wherein the isotope label of the isotopically labeled bile acid is ²H, ³H, ¹³C, ¹⁴C, or ¹⁸O.

10. The method of claim 9 wherein ¹³C₂ labeled cholesterol in a lipid emulsion and ²H₄-cholic acid are administered to said living system.

11. The method of claim 1 further including measuring the contribution of de novo cholesterol synthesis to bile acids, comprising:
i) administering an isotopically labeled cholesterol precursor to said living system wherein said precursor has a defined label concentration;
ii) obtaining a biological sample from said living system wherein said sample comprises labeled bile acid, excreted neutral sterol or blood cholesterol;
iii) measuring the isotopic content or pattern or rate of change of said isotopic content or pattern of said labeled bile acid, excreted neutral sterol or blood cholesterol; and
iv) comparing the isotopic content or pattern or rate of change of said isotopic content or pattern of the bile acids, neutral sterols or cholesterol to the label concentration of the stable isotope-labeled cholesterol precursor to determine the fraction of cholesterol, neutral sterol or bile acids that are derived from newly synthesized cholesterol to measure the contribution by de novo cholesterol synthesis to said bile acid, neutral sterol or cholesterol.

12. The method of claim 11 wherein the isotopically labeled cholesterol precursor is deuterated water.

13. The method of claim 1 wherein the sample is a stool and the total content of neutral sterols and bile acids excreted by a subject per unit time is measured by comparison to an internal standard detected in the stool that was administered orally to the subject.

14. The method of claim 13 wherein the internal standard is sitostanol.

15. A method for determining the molecular flux rate of the plasma component of reverse cholesterol transport (RCT) in a living system, said method comprising:
a) administering a stable, isotopically labeled cholesterol molecule or a stable isotopically labeled cholesterol-related molecule to the living system;
b) obtaining a sample from said living system wherein said sample comprises an in vivo conversion product of said isotopically labeled cholesterol molecule or said isotopically labeled cholesterol-related molecule;
c) measuring isotopic content, isotopic pattern, rate of change of isotopic content, or isotopic pattern of the in vivo conversion product; and
d) calculating the rate of dilution of the isotopically labeled cholesterol molecule or the isotopically labeled cholesterol-related molecule to determine the molecular flux rate of the plasma component of reverse cholesterol transport (RCT) in the living system.

16. The method of claim 15, wherein said sample is a stool, urine or blood sample.

17. The method of claim 15, wherein the isotope label of the isotopically labeled cholesterol molecule or isotopically labeled cholesterol-related molecule is ²H, ³H, ¹³C, ¹⁴C or ¹⁸O.

18. The method of claim 15 wherein said living system is a human or a rodent.

19. The method of claim 15 wherein ¹³C₂ labeled cholesterol in complex with HDL is administered to said living system.

20. The method of claim 19 wherein said sample is a blood sample comprising HDL, VLDL and LDL and the isotopic content of HDL-cholesterol, LDL-cholesterol ester, and VLDL cholesterol ester are determined by GCC-IRMS.

21. A method for determining the rate of appearance of cholesterol in blood, or cholesterol tissue efflux rate, in a living system, said method comprising:
a) administering an isotopically labeled cholesterol molecule or isotopically labeled cholesterol-related molecule intravenously to the living system at a known or measurable rate, said administration rate being sufficient to result in an accumulation of detectable levels of labeled free cholesterol in said living system;
b) obtaining samples from said living system wherein said samples comprise said labeled, free cholesterol molecule;
c) measuring isotopic content, isotopic pattern, rate of change of isotopic content, or isotopic pattern of the labeled, free cholesterol molecule; and
d) calculating the rate of appearance of cholesterol in blood in the living system by comparing the isotopic content, isotopic pattern, rate of change of isotopic content, or isotopic pattern of the labeled, free cholesterol molecule to the rate of administration of the isotopically labeled cholesterol molecule or isotopically labeled cholesterol-related molecule.

22. The method of claim 21, wherein said living system is a human or a rodent.

23. The method of claim 21, wherein the rate of appearance of cholesterol in blood in the living system is calculated by isotope dilution, according to the plateau principle, by establishing the existence of isotopic plateau, by inferring the isotopic plateau value or by extrapolating the isotopic plateau value.

24. A method for calculating the rate flux of cholesterol in a living system, said method comprising,
a) measuring the rate of appearance of cholesterol in blood by:
i) administering ¹³C-, ²H or ¹⁸O-labeled cholesterol in a lipid emulsion intravenously to a living system at an administration rate sufficient to result in an accumulation of detectable levels of labeled, free cholesterol in said living system;
ii) obtaining samples from said living system wherein said samples comprise a labeled, free cholesterol molecule;
iii) measuring isotopic content, isotopic pattern, rate of change of isotopic content, or isotopic pattern of the labeled, free cholesterol molecule; and
iv) calculating the rate of appearance of cholesterol in blood in the living system by comparing the isotopic content, isotopic pattern, rate of change of isotopic content, or isotopic pattern of the labeled, free cholesterol molecule to the rate of administration of the ¹³C-, ²H or ¹⁸O-labeled cholesterol;
b) measuring the percentage recovery of the hepatic or excretory arm of reverse cholesterol transport (RCT) by:
i) administering an isotopically labeled cholesterol molecule or isotopically labeled cholesterol-related molecule to the living system;
ii) obtaining a sample from said living system wherein said sample comprises one or more isotopically labeled bile acids or excreted neutral sterols from the living system;
iii) measuring isotopic content, isotopic pattern, rate of change of isotopic content, or isotopic pattern of the isotopically labeled bile acids or excreted neutral sterols; and
iv) calculating the rate of incorporation or transfer of the isotopically labeled cholesterol-related molecule into said bile acids or excreted neutral sterols to determine the percentage recovery of the hepatic or excretory component of RCT in the living system; and
c) calculating the rate of flux of cholesterol in the living system by multiplying the rate of appearance of cholesterol in blood from a) iii) by the percentage recovery of the hepatic or excretory arm of RCT from b) iv).

25. The method of claim 24 wherein said living system is a human.

26. A method of assessing the effect of a candidate agent and/or dietary modification on the risk for and rate of development of atherosclerosis in a living system, the method comprising:
a) calculating the rate of flux of cholesterol in the living system by the method of claim 24;
b) administering said candidate agent to said living system and/or modifying the diet of said living system;
c) calculating the rate of flux of cholesterol in the living system by the method of claim 24; and
d) comparing the difference between the cholesterol flux rates of steps a) and c) to assess the effect of the candidate agent and/or the dietary modification on atherosclerosis.

27. A method of assessing the effect of a candidate agent and/or dietary modification on the risk for and rate of development of atherosclerosis in a living system, the method comprising:
a) determining the molecular flux rate of the hepatic or excretory component of reverse cholesterol transport (RCT) in a living system by the method of claim 1;
b) administering said candidate agent to said living system and/or modifying the diet of said living system;
c) determining the molecular flux rate of the hepatic or excretory component of reverse cholesterol transport (RCT) in the living system by the method of claim 1; and
d) comparing the difference between the molecular rate fluxes of steps a) and c) to assess the effect of the candidate agent and/or the dietary modification on atherosclerosis.

28. A method of assessing the effect of a candidate agent and/or dietary modification on the risk for and rate of development of atherosclerosis in a living system, the method comprising:
a) determining the molecular flux rate of the plasma component of reverse cholesterol transport (RCT) in a living system by the method of claim 12;
b) administering said candidate agent to said living system and/or modifying the diet of said living system;
c) determining the molecular flux rate of the plasma component of reverse cholesterol transport (RCT) in a living system by the method of claim 12; and
d) comparing the difference between the molecular rate fluxes of steps a) and c) to assess the effect of the candidate agent and/or the dietary modification on atherosclerosis.

29. A method of assessing the effect of a candidate agent and/or dietary modification on the risk for and rate of development of atherosclerosis in a living system, the method comprising:
a) determining the rate of appearance of cholesterol in a living system by the method of claim 21;
b) administering said candidate agent to said living system and/or modifying the diet of said living system;
c) determining the rate of appearance of cholesterol in a living system by the method of claim 21; and
d) comparing the difference between the molecular rate fluxes of steps a) and c) to assess the effect of the candidate agent and/or the dietary modification on atherosclerosis.

30. A method for correcting the recovery of labeled cholesterol in fecal sterols for the efflux/influx rate of cholesterol across tissues, said method comprising:
a) measuring the percentage recovery of the hepatic or excretory arm of RCT by
i) administering an isotopically labeled cholesterol molecule or isotopically labeled cholesterol-related molecule to the living system at a known or measurable rate;
ii) obtaining a sample from said living system wherein said sample comprises one or more isotopically labeled cholesterol molecules, bile acids or excreted neutral sterols from the living system;
iii) measuring isotopic content, isotopic pattern, rate of change of isotopic content, or isotopic pattern of the isotopically labeled cholesterol molecules, bile acids or excreted neutral sterols; and
iv) calculating the rate of incorporation or transfer of the isotopically labeled cholesterol molecule or isotopically labeled cholesterol-related molecule into said cholesterol molecules, bile acids or excreted neutral sterols to determine the molecular flux rate of the hepatic or excretory component of RCT in the living system
b) measuring the rate of appearance of cholesterol in blood by
i) administering an isotopically labeled cholesterol molecule or isotopically labeled cholesterol-related molecule intravenously to the living system at a known or measurable rate, said administration rate being sufficient to result in an accumulation of detectable levels of labeled free cholesterol in said living system;
ii) obtaining samples from said living system wherein said samples comprise said labeled, free cholesterol molecule;
iii) measuring isotopic content, isotopic pattern, rate of change of isotopic content, or isotopic pattern of the labeled, free cholesterol molecule; and
iv) calculating the rate of appearance of cholesterol in blood in the living system by comparing the isotopic content, isotopic pattern, rate of change of isotopic content, or isotopic pattern of the labeled, free cholesterol molecule to the rate of administration of the isotopically labeled cholesterol molecule or isotopically labeled cholesterol-related molecule.
c) correcting the recovery of labeled cholesterol in fecal sterols for the efflux/influx rate of cholesterol across tissues by multiplying the rate of appearance of cholesterol of step b) by the percentage recovery of step a).

31. A kit for calculating the rate of RCT flux in a living system, comprising:
a) one or more isotopically labeled HDL particles, isotopically labeled cholesterol molecules, isotopically labeled cholesterol precursors, or isotopically labeled bile acids; and
b) instructions for use of the kit;
wherein the kit is used to calculate the rate of flux of cholesterol in the living system.

32. The kit of claim 31, further comprising a tool for administering the isotopically labeled HDL particles or labeled bile acids.

33. The kit of claim 31, further comprising an instrument for collecting a biological sample from the living system.
